(19) United States
(12) Patent Application Publication
(10) Pub. No.: US 2017/0296650 A1
(43) Pub. Date: Oct. 19, 2017

(54) COMBINATION OF HUMAN CYTOMEGALOVIRUS NEUTRALIZING ANTIBODIES

(71) Applicant: Novartis AG, Basel (CH)

(72) Inventors: Adam FEIRE, Hull, MA (US); Yinuo PANG, Sudbury, MA (US); Peter PERTEL, Brookline, MA (US); Jing YU, Watertown, MA (US)

(21) Appl. No.: 15/516,655

(22) PCT Filed: Oct. 7, 2015
(86) PCT No.: PCT/IB2015/057664

§ 371 Date: Apr. 3, 2017

Related U.S. Application Data
(60) Provisional application No. 62/061,378, filed on Oct. 8, 2014.
(60) Provisional application No. 62/204,653, filed on Aug. 13, 2015.

(51) Int. Cl. A61K 39/245; C07K 16/08; A61K 39/00

(52) U.S. Cl. CPC A61K 39/245; C07K 16/088; C07K 2317/92; C07K 2317/76; A61K 2039/507; C07K 2317/21; C07K 2317/94; C07K 2317/33

(57) ABSTRACT

The disclosure relates to the use of a combination of antibodies or antigen binding fragments thereof to hCMV; and to dosages, ratios and minimum trough serum concentrations of the antibodies. The combination is useful for the neutralization of hCMV, for example, in pregnant, immunocompromised or immunosuppressed patients undergoing bone marrow and organ transplants with a low occurrence of viral resistance.

### BACKGROUND

Human cytomegalovirus (hCMV) is a widely distributed pathogen that may cause severe pathology in immunosuppressed adults and upon infection of the fetus and has been implicated in chronic diseases such as atherosclerosis. Ho 2008 Med. Microbiol. Immunol. 197: 65-73. hCMV infects multiple cell types including fibroblasts, endothelial, epithelial and hematopoietic cells, Plachter et al. 1996 Adv. Virus Res. 46:195-261. In vitro propagated attenuated strains of hCMV, which are being developed as candidate vaccines, have lost the tropism for endothelial cells, while retaining the capacity to infect fibroblasts, Gerna et al. 2002 J. Med. Virol. 66:335-339. Two viral glycoprotein complexes are believed to control the cellular tropism of hCMV. A complex of glycoproteins gH, gL and gO appears to be required for infection of fibroblasts, while a complex of gH, gL and proteins encoded by the UL131-UL128 genes is implicated in infection of endothelial cells, epithelial cells and dendritic cells Gerna et al. 2002 J. Med. Virol. 66:335-339; Adler, et al. 2006. J. Gen. Virol. 87:2451-2460; Gerna, et al. 2005. J. Gen. Virol.

86:275-284; Hahn, et al. 2004. J. Virol. 78:10023-10033; Patrone, et al. 2005. J. Virol. 79:8361-8373; Wang, et al. 2005. Proc. Natl. Acad. Sci. USA 102:18153-18158; Wang, et al. 2005. J. Virol. 79:10330-10338.

Therapies available to prevent or treat HCMV disease in transplant recipients, including ganciclovir, cidofovir, and foscarnet, are all are associated with serious toxicities. Currently, there are no approved therapies to prevent or treat congenital hCMV. Biron 2006 Antiviral Res. 71: 154-63.

Hyperimmune globulins, in the form of a polyclonal IgG preparation purified from human plasma pools, are already commercialized for the prophylaxis of hCMV disease associated with transplantation and recent evidence indicates that they have therapeutic effect in pregnant women, Nigro et al. 2005. N. Engl. J. Med. 353:1350-1362. This therapeutic approach is limited by the low amount of neutralizing antibody that can be transferred, and for this reason the availability of human antibodies (such as human monoclonal antibodies) with high neutralizing capacity would be highly desirable. Although some antibodies to gH, gB and UL128 and UL130 gene products have demonstrated in vitro neutralizing activities (Wang, et al. 2005. Proc. Natl. Acad. Sci. USA 102:18153-18158; Borucki et al. 2004, Antiviral Res. 64:103-111; McLean et al. 2005. J Immunol, 174:4768-4778), and an antibody to gH was evaluated in clinical trials (that were discontinued due to lack of therapeutic effects), the neutralizing potency of these antibodies is modest. Boeckh et al. 2001 Biol. Blood Marrow Transplant 7: 343-51; and Manley et al. 2011 Cell Host Microbe 10: 197-209. Neutralization by these antibodies was observed at antibody concentrations ranging from 0.5 to 20 μg/ml. Further, the current methods typically measure the neutralizing potency of anti-hCMV antibodies using fibroblasts as target cells. However, hCMV is also known to cause pathology by infecting other cell types such as endothelial, epithelial cells and leukocytes. The antibodies described in Wang, D., and T. Shenk. 2005. Proc. Natl. Acad. Sci. USA 102:18153-18158, to UL128 and UL130 show very low potency in neutralizing infection of endothelial cells.

There is therefore a need for antibodies or combinations thereof that neutralize hCMV infection, particularly hCMV infection of non-fibroblast target cells, with high potency, as well as the elucidation of the target(s) to which such antibodies bind.

### SUMMARY OF INVENTION

The disclosure provides a composition comprising a combination of antibodies or antigen-binding fragments thereof, wherein the antibodies or fragments neutralize hCMV infection with high potency and comprise the CDR sequences of antibodies 7H3 and 4I22, which were isolated from different immortalized B cells. In some embodiments of the disclosure, the disclosure provides specific dosages of the two antibodies or antigen binding fragments. In some embodiments, the disclosure provides minimum trough serum concentrations for the antibodies or fragments. In some embodiments, the disclosure provides compositions comprising specific ratios of the two antibodies or antigen binding fragments to hCMV. The disclosure also provides methods of use of these compositions. The use of the combination decreases the development or risk of development of viral resistance to either antibody or fragment.

In one embodiment, the disclosure provides a method of neutralizing hCMV infection, comprising the steps of: (a) administering a dose (e.g., by injection or infusion) of a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332, and the CDRL1, CDRL2, and CDRL3 sequences of SEQ ID NOs: 319, 320, and 321, respectively; and (b) administering a dose of a second antibody or antigen binding fragment thereof, which binds to a 5-member (pentameric) complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively; wherein the first antibody or antigen binding fragment thereof is administered at a dosage of about 1 to about 50 mg/kg body weight, and the second antibody or antigen binding fragment thereof is administered at a dosage of about 0.1 to about 5.0 mg/kg body weight, wherein steps (a) and (b) can be performed simultaneously or in any order, and wherein steps (a) and/or (b) can optionally be repeated to administer multiple doses. In some embodiments, in (a) the CDRH3 sequence is SEQ ID NO: 332. In some embodiments, in (a) the CDRH3 sequence is SEQ ID NO: 318. In some embodiments, the ratio of the dose of the first antibody or fragment to the second antibody or fragment is about 10:1. In some embodiments, the ratio of the first antibody or fragment to the second antibody or fragment is between about 7.5:1 and about 12.5:1. In some embodiments, the ratio is about 20:1. In some embodiments, the ratio is about 15:1. In some embodiments, the ratio is about 12.5:1. In some embodiments, the ratio is about 7.5:1. In some embodiments, the ratio is about 5:1. In some embodiments, the ratio is about 4:1. In some embodiments, the ratio is about 3:1. In some embodiments, the ratio is about 2:1. In some embodiments, the ratio is about 2:1 to about 20:1. In some embodiments, the ratio is about 5:1 to about 20:1. In one embodiment of this method, the first antibody or antigen binding fragment thereof is administered at a dosage of about 2.5 to about 25 mg/kg body weight, and the second antibody or antigen binding fragment thereof is administered at a dosage of about 0.25 to about 2.5 mg/kg body weight. In one embodiment of this method, the first antibody or antigen binding fragment thereof is administered at a dosage of about 5 to 10 mg/kg body weight, and the second antibody or antigen binding fragment thereof is administered at a dosage of about 0.5 to about 1 mg/kg body weight. In one embodiment of this method, the first antibody or antigen binding fragment thereof is administered at a dosage of about 5 mg/kg body weight, and the second antibody or antigen binding fragment thereof is administered at a dosage of about 0.5 mg/kg body weight. In various embodiments of this method, the doses are administered intraperitoneally, orally, subcutaneously, intramuscularly, topically or intravenously. In some embodiments, the first and second antibody or fragment are in lyophilized form. In some embodiments, the first and second antibody or fragment are reconstituted prior to injection or infusion. In some embodiments, the first and second antibody or fragment are reconstituted in a pharmaceutical carrier. In some embodiments, the pharmaceutical carrier is for injection or infusion into an immunocompromised or immunosuppressed subject. In some embodiments, the pharmaceutical carrier is for injection or infusion into a pregnant subject. In some embodiments, the doses are administered intraperitoneally, orally, subcutaneously, intramuscularly, topically or intravenously. In some embodiments, the doses of the first and second antibody or antigen binding fragment thereof are administered on the same day. In some embodiments, the doses are each administered as a single dosage. In one embodiment of this method, the doses of the first and second antibody or antigen binding fragment thereof are administered on the same day. In one embodiment of this method, the doses are each administered as a single dosage. In one embodiment of this method, the doses are each administered as multiple doses. In various embodiments of this method, the doses are administered about every week, every two weeks, every three weeks, every four weeks, every month, ever month and a half, or every two months. In various embodiments of this method, the doses are administered over a period of about six months, about 9 months, or about one year. In one embodiment of this method, the method further comprises the step (c) of determining an efficacious range for the first and/or second antibody or antigen binding fragment thereof in the blood of the subject, wherein steps (a), (b) and (c) can be performed simultaneously or in any order. In one embodiment of this method, the method further comprises the step (d) of monitoring the subject for the level of first and/or second antibody or antigen binding fragment thereof in the blood of the subject, wherein step (d) is performed after steps (a), (b) and (c). In one embodiment of this method, the method further comprises the step (e) of administering or altering the dosage of the first and/or second antibody or antigen binding fragment administered to the subject, in order to maintain the first and/or second antibody or antigen binding fragment within the efficacious range in the blood of the subject, wherein step (e) is performed after step (d). In one embodiment of this method, the efficacious range is a minimum trough serum concentration of at least about 7.4 μg [microgram] /ml for the first antibody; and a minimum trough serum concentration of at least about 0.74 μg [microgram] /ml for the second antibody. The use of the combination of the first and second antibody or fragment decreases the development or risk of development of viral resistance to either antibody or fragment.

In one embodiment, the disclosure provides a method of neutralizing hCMV infection, comprising the steps of: (a) administering a dose of a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332, and the CDRL1, CDRL2, and CDRL3 sequences of SEQ ID NOs: 319, 320, and 321, respectively; and (b) administering a dose of a second antibody or antigen binding fragment thereof, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively; wherein steps (a) and (b) can be performed simultaneously or in any order, and wherein steps (a) and/or (b) can optionally be repeated to administer multiple doses, and wherein the ratio of the dose first antibody or fragment to the second antibody or fragment is between about 7.5:1 and about 12.5:1. In one embodiment of this method, the ratio of the dose of the first antibody or fragment to the second antibody or fragment is about 10:1. In some embodiments, the ratio is about 7.5:1. In some embodiments, the ratio is about 12.5:1. In some embodiments, the ratio is about 5:1. In some embodiments, the ratio is about 15:1. In some embodiments, the ratio is about 20:1. In some embodiments, the ratio is about 5:1 to about 20:1. The use of the combination of the first and second antibody or fragment decreases the development or risk of development of viral resistance to either antibody or fragment. In some embodiments, in (a) the CDRH3 sequence is SEQ ID NO: 332. In some embodiments, in (a) the CDRH3 sequence is SEQ ID NO: 318.

In one embodiment, the disclosure provides a method of neutralizing hCMV infection, comprising the steps of: (a) administering one or more doses of a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively; wherein the one or more doses are sufficient to maintain a minimum trough serum concentration of at least about 7.4 μg [microgram] /ml; and (b) administering one or more doses of a second antibody or antigen binding fragment thereof, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively; wherein the one or more doses are sufficient to maintain a minimum trough serum concentration of at least about 0.74 μg [microgram] /ml; wherein steps (a) and (b) can be performed simultaneously or in any order. The use of the combination of the first and second antibody or fragment decreases the development or risk of development of viral resistance to either antibody or fragment. In some embodiments, in (a) the CDRH3 sequence is SEQ ID NO: 332. In some embodiments, in (a) the CDRH3 sequence is SEQ ID NO: 318.

In one embodiment, the disclosure provides a composition comprising: (a) a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively; and (b) a second antibody or antigen binding fragment thereof, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively; wherein the ratio of the first antibody or fragment to the second antibody or fragment is between about 7.5:1 and about 12.5:1. In one embodiment of the composition, the ratio of the dose first antibody or fragment to the second antibody or fragment is about 10:1. In some embodiments, the ratio is about 7.5:1. In some embodiments, the ratio is about 12.5:1. In some embodiments, the ratio is about 5:1. In some embodiments, the ratio is about 15:1. In some embodiments, the ratio is about 20:1. In some embodiments, the ratio is about 5:1 to about 20:1. The use of the combination of the first and second antibody or fragment decreases the development or risk of development of viral resistance to either antibody or fragment. In some embodiments, in (a) the CDRH3 sequence is SEQ ID NO: 332. In some embodiments, in (a) the CDRH3 sequence is SEQ ID NO: 318. In some embodiments, the ratio of the dose first antibody or fragment to the second antibody or fragment is about 10:1. In some embodiments, the ratio is about 7.5:1. In some embodiments, the ratio is about 12.5:1. In some embodiments, the ratio is about 5:1. In some embodiments, the ratio is about 15:1. In some embodiments, the ratio is about 20:1. In some embodiments, the ratio is about 5:1 to about 20:1. In some embodiments, the first and second antibody or fragment are in lyophilized form. In some embodiments, the first and second antibody or fragment are reconstituted prior to injection or infusion. In some embodiments, the first and second antibody or fragment are reconstituted in a pharmaceutical carrier. In some embodiments, the pharmaceutical carrier is for injection or infusion into an immunocompromised subject. In some embodiments, the pharmaceutical carrier is for injection or infusion into a pregnant subject. In some embodiments, the disclosure pertains to a kit comprising the composition and a package insert comprising instructions for administration of the composition for treating hCMV infection.

### BRIEF DESCRIPTION OF THE FIGURE

FIG. 1 shows the mean serum concentration, in semilogarithmic view, of 7H3 (A) and 4I22 (B) versus time, in humans. Cohorts are indicated.

Cohort 1: 7H3 (1 mg/kg)/4I22 (0 mg/kg)

Cohort 2: 7H3 (0 mg/kg)/4I22 (0.1 mg/kg)

Cohort 3: 7H3 (1 mg/kg)/4I22 (0.1 mg/kg)

Cohort 4: 7H3 (5 mg/kg)/4I22 (0.5 mg/kg)

Cohort 5: 7H3 (20 mg/kg)/4I22 (2 mg/kg)

Cohort 6: 7H3 (50 mg/kg)/4I22 (5 mg/kg)

### DETAILED DESCRIPTION OF THE INVENTION

The disclosure provides dosages, ratios and minimum serum trough concentrations of combination of antibodies or antigen-binding fragments thereof, wherein the antibodies or fragments neutralize hCMV infection with high potency, and comprise the CDR sequences of 7H3 and 4I22, which were isolated from different immortalized B cells. The disclosure also provides methods of use of this combination of antibodies or antigen-binding fragments thereof. In various embodiments, the disclosure provides a combination of: an antibody or antigen binding fragment thereof comprising the CDR sequences of antibody 7H3, e.g., the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively, wherein the antibody or fragment binds to and/or inhibits hCMV glycoprotein gB; and an antibody or antigen binding fragment thereof comprising the CDR sequences of antibody 4I22, e.g., the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively, wherein the antibody or fragment binds to and/or inhibits a 5-member complex consisting of hCMV glycoproteins gH, gL, UL128, UL130 and UL131A.

As used herein, the terms “fragment,” “antigen-binding fragment,” “antigen binding fragment” and “antibody fragment” and the like are used interchangeably to refer to any fragment of an antibody of the disclosure that retains the antigen-binding activity of the antibodies. Example antibody fragments include, but are not limited to, a single chain antibody, Fab, Fab′, F(ab′)2, Fv or scFv. As a non-limiting example, an antigen-binding fragment of an antibody can retain the CDR sequences of the antibody from which it is derived.

As used herein, the term “high potency” is used to refer to an antibody or an antigen binding fragment thereof (or combination of antibodies or antigen binding fragments thereof) that substantially neutralizes hCMV infection. In various embodiments, the antibody or fragment or combination neutralizes hCMV infection with an IC₉₀ of less than about 2 μg/ml, (i.e. the concentration of antibody required for 90% neutralisation of a clinical isolate of hCMV is about 2 μg/ml or less, for example 1.9, 1.8, 1.75, 1.7, 1.6, 1.5, 1.4, 1.3, 1.25, 1.2, 1.15, 1.1, or 1.05 μg/ml or less). In one embodiment, the antibody of the present disclosure, or antigen binding fragment thereof, has an IC₉₀ of 1 μg/ml or less (i.e. 0.95, 0.9, 0.85, 0.8, 0.75, 0.7, 0.6, 0.5, 0.4, 0.3, 0.2, 0.1, 0.05, 0.01 μg/ml or less). In another embodiment, the antibody of the present disclosure, or antigen binding fragment thereof, has an IC₉₀ of 0.16 μg/ml or less (i.e. 0.15, 0.125, 0.1, 0.075, 0.05, 0.025, 0.02, 0.015, 0.0125, 0.01, 0.0075, 0.005, 0.004, 0.003, 0.002 μg/ml or less). In another embodiment, the antibody can neutralize hCMV infection at a concentration of 0.016 μg/ml or less (i.e. at 0.015, 0.013, 0.01, 0.008, 0.005, 0.003, 0.002, 0.001, 0.0005 μg/ml or less). This means that only very low concentrations of antibody are required for 90% neutralisation of a clinical isolate of hCMV in vitro compared to the concentration of known antibodies, e.g., MSL-109, 8F9 or 3E3, required for neutralisation of the same titre of hCMV. Potency can be measured using a standard neutralisation assay as known to one of skill in the art. The potencies of antibodies 7H3 and 4I22 and combinations thereof are described herein.

In another embodiment, the disclosure provides a combination comprising an antibody, or an antigen binding fragment thereof, that binds to an epitope formed by the hCMV proteins

UL130 and UL131A, and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope formed by these proteins by 4I22 is shown in Table 6.

In another embodiment, the disclosure provides a combination comprising an antibody, or an antigen binding fragment thereof, that binds to an epitope formed by the hCMV proteins UL128, UL130 and UL131A, and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope formed by these proteins by 4I22 is shown in Table 6.

In another embodiment, the disclosure provides a combination comprising an antibody, or an antigen binding fragment thereof, that binds to an epitope formed by the hCMV proteins gH, UL128, UL130 and UL131A, and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope formed by these proteins by 4I22 is shown in Table 6.

In another embodiment, the disclosure provides a combination comprising an antibody, or an antigen binding fragment thereof, that binds to an epitope formed by the hCMV proteins gL, UL128, UL130 and UL131A, and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope formed by these proteins by 4I22 is shown in Table 6.

In another embodiment, the disclosure provides a combination comprising an antibody, or an antigen binding fragment thereof, that binds to an epitope formed by the hCMV proteins gH, gL, UL128 and UL130, and UL131A, and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope formed by these proteins by 4I22 is shown in Table 6.

In yet another embodiment, the disclosure provides a combination comprising an antibody, or an antigen binding fragment thereof, that binds to an epitope in the hCMV gB protein and neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of an epitope in this protein by 7H3 is shown in Table 6.

In various embodiments, the disclosure provides a combination comprising: an antibody or an antigen binding fragment thereof, that binds to an epitope in the hCMV gB protein; and an antibody or an antigen binding fragment thereof, that binds to an epitope formed by the hCMV proteins UL130 and UL131A; UL128, UL130 and UL131A; gH, UL128, UL130 and UL131A; gL, UL128, UL130, and UL131A; or gH, gL, UL128, UL130, and UL131A, wherein the combination neutralizes hCMV infection with an IC₉₀ of less than about 2 μg/ml, for example 1.9, 1.8, 1.75, 1.7, 1.6, 1.5, 1.4, 1.3, 1.25, 1.2, 1.15, 1.1, 1.05, 1, 0.95, 0.9, 0.85, 0.8, 0.75, 0.7, 0.6, 0.5, 0.4, 0.3, 0.2, 0.15, 0.125, 0.1, 0.075, 0.05, 0.025, 0.02, 0.015, 0.0125, 0.01, 0.0075, 0.005, 0.004, 0.003, 0.002 0.001, 0.0005 μg/ml or less. Binding of 7H3 and 4I22 to epitopes in and/or formed by these proteins is shown in Table 6.

### Antibodies of the Disclosure

The disclosure provides combinations of antibodies having particularly high potency in neutralizing hCMV. As used herein, the terms “antibody that neutralizes”, “antigen binding fragment thereof that neutralizes” and the like refer to one that prevents, reduces, delays or interferes with the ability of a pathogen, e.g., hCMV, to initiate and/or perpetuate an infection in a host. The combinations of antibodies and antigen-binding fragments thereof of the disclosure are able to neutralize hCMV infection of several kinds of cells. In one embodiment, a combination of antibodies according to the disclosure neutralizes infection of epithelial cells, retinal cells, endothelial cells, myeloid cells and dendritic cells. The combinations of antibodies of the disclosure may also neutralize hCMV infection of fibroblasts and mesenchymal stromal cells. These combinations of antibodies can be used as prophylactic or therapeutic agents upon appropriate formulation, or as a diagnostic tool, as described herein.

The disclosure thus provides a method of neutralizing hCMV infection, e.g., a method of preventing hCMV infection, and/or reducing, delaying or interfering with the ability of hCMV to initiate and/or perpetuate an infection, and/or inhibiting hCMV in a subject, such as a human. The method comprises the steps of administering an efficacious amount of a combination of two or more hCMV neutralizing antibodies or antigen binding fragments thereof. As a non-limiting example, the combination comprises a first antibody or fragment comprising the CDR sequences of 7H3 and a second antibody or fragment comprising the CDR sequences of 4I22. In various embodiments, the first antibody or antigen binding fragment thereof is administered at a dosage of about 1 -50, 2.5 to 25, 5 to 20, 5 to 10, or 5 mg/kg body weight. In various embodiments, the second antibody or antigen binding fragment thereof is administered at a dosage of about 0.1 to 5.0, 0.25 to 2.5, .5 to 2, 0.5 to 1, or 0.5 mg/kg body weight. In various embodiments, the dosages of the first and second antibodies or fragments are 5 and 0.5 mg/kg body weight, respectively. In various embodiments, the ratios of the first antibody or fragment : second antibody or fragment, as administered or as included in a composition, are between about 7.5:1 and about 12.5:1; about 10:1, or 10:1. In some embodiments, the ratio is about 7.5:1. In some embodiments, the ratio is about 12.5:1. In some embodiments, the ratio is about 5:1. In some embodiments, the ratio is about 15:1. In some embodiments, the ratio is about 20:1. In some embodiments, the ratio is about 5:1 to about 20:1. In various embodiments, the dosages of the first and second antibody or fragment and/or dosing frequency are sufficient to sufficient to maintain a minimum trough serum concentration of at least about 7.4 μg/ml and 0.74 μg/ml, respectively, of the first and second antibody or fragment. In various embodiments, the dosages are administered intraperitoneally, orally, subcutaneously, intramuscularly, topically or intravenously. In various embodiments, the dosages of the first and second antibody or antigen binding fragment thereof are administered simultaneously, on the same day, and/or in any order.

In various embodiments, the doses are administered as a single dose or multiple doses (e.g., a single dose followed by additional doses). In various embodiments pertaining to multiple doses, the doses are administered about every week, every two weeks, every three weeks, every four weeks, every month, ever month and a half, or every two months. In various embodiments pertaining to multiple doses, the dosages are administered about every two weeks or four weeks. In various embodiments pertaining to multiple doses, the dosages are administered over a period of about six months, about 9 months, or about one year.

In various embodiments, the method further comprises a step (c) of determining an efficacious range for the first and/or second antibody or antigen binding fragment thereof in the blood of the subject, wherein steps (a), (b) and (c) can be performed simultaneously or in any order. In various embodiments, the method further comprises a step (d) of monitoring the subject for the level of first and/or second antibody or antigen binding fragment thereof in the blood of the subject, wherein step (d) is performed after steps (a), (b) and (c). In various embodiments, the method further comprises a step (e) of administering or altering the dosage of the first and/or second antibody or antigen binding fragment administered to the subject, in order to maintain the first and/or second antibody or antigen binding fragment within the efficacious range in the blood of the subject, wherein step (e) is performed after step (d). In various embodiments, the efficacious range is a range which is at least the minimum trough serum concentration of at least about 7.4 μg/ml for the first antibody, and the minimum trough serum concentration of at least about 0.74 μg/ml for the second antibody.

The antibodies of the disclosure may be monoclonal antibodies, human antibodies, or recombinant antibodies. In one embodiment, the antibodies of the disclosure are monoclonal antibodies, e.g., human monoclonal antibodies. The disclosure also provides fragments of the antibodies of the disclosure, particularly fragments that retain the antigen-binding activity of the antibodies and neutralize hCMV infection. Although the specification, including the claims, may, in some places, refer explicitly to antibody fragment(s), variant(s) and/or derivative(s) of antibodies, it is understood that the term “antibody” or “antibody of the disclosure” includes all categories of antibodies, namely, antibody fragment(s), variant(s) and derivative(s) of antibodies.

In one embodiment, the antibodies of the disclosure and antigen binding fragments thereof bind to one or more hCMV proteins. The antibodies of the disclosure may bind to an epitope formed by a single hCMV protein or by a combination of two or more hCMV proteins. Example hCMV proteins include, but are not limited to, products of viral genes UL55 (envelope glycoprotein B, “gB”), UL75 (envelope glycoprotein H, “gH”), UL100 (glycoprotein M, “gM”), UL73 (glycoprotein N, “gN”), UL115 (glycoprotein L, “gL”), UL74 (glycoprotein O, “gO”), UL128 (glycoprotein UL128, “UL128”), UL130 (glycoprotein UL130, “UL130”) or UL131A (glycoprotein UL131A, “UL131A”). In one embodiment, the antibodies of the disclosure bind to an epitope formed by a single hCMV protein, e.g., gB, which is bound by 7H3. In another embodiment, the antibodies bind to an epitope formed by the combination of 2, 3, or more hCMV proteins, e.g., the 5-protein complex, which is bound by 4122.

hCMV glycoproteins have important roles in viral replication. The first step in viral replication is the entry process, whereby hCMV binds to and fuses with the host cell (Compton 2004 Trends Cell. Biol. 14: 5-8). After entry, the nucleocapsid containing the DNA genome is transported to the cell nucleus, either initiating viral replication and production of progeny virions or establishing latency. In contrast to many viruses, hCMV entry is a complex series of interactions between multiple viral glycoprotein complexes and host cell surface receptors. hCMV initially attaches to host cells through low affinity interactions of a viral heterodimer consisting of glycoproteins gM and gN with cell surface heparan sulfate proteoglycans (Kari and Gehrz 1992 J. Virol. 66: 1761-4). Subsequent higher affinity virus binding requires interaction of glycoprotein gB with yet unknown host receptors, an interaction which triggers signal transduction cascades that activate growth factor receptors (Wang et al 2003 Nature 424: 456-61, Soroceanu et al 2008 Nature 455: 391-5). After binding, gB interacts with cellular integrins to trigger fusion of the virus envelope with the cell membrane (Feire et al 2004 Proc. Natl. Acad. Sci. USA 101: 15470-5, Feire et al 2010 J. Virol. 84: 10026-37). Fusion also requires the interaction of unknown host factors with one of two viral glycoprotein complexes, both of which contain glycoproteins gH and gL.

Entry into different cell types is mediated by different hCMV glycoproteins. In contrast to gB, which is required for entry into all physiologically relevant cell types, gH and gL form two different complexes that mediate entry into distinct cell populations. A 3-member complex, consisting of gH, gL, and gO, is essential for entry into fibroblast cells while a 5-member complex, consisting of glycoproteins gH, gL, UL128, UL130, and UL131A, is essential for entry into myeloid, epithelial, and endothelial cells (Hahn et al 2004 J. Virol. 78: 10023-33, Wang and Shenk 2005 Proc. Natl. Acad. Sci. USA 102: 18153-8). Targeting the viral glycoproteins required for hCMV to infect different cell types is important because disease pathogenesis presumably requires hCMV to infect different cell types. Infection of endothelial and hematopoietic cells appears to facilitate the systemic spread of virus while infection of epithelial cells and fibroblasts appears to contribute to high level replication of virus (Sinzger et al 2008 Curr. Top. Microbiol. Immun. 325: 63-83). In addition to playing essential roles in mediating viral entry into host cells, gB and the 5-member complex are both required for hCMV-induced cell-cell fusion. Such fusion allows the transfer of virus between monocytes and endothelial cells, and potentially enhances the systemic dissemination of virus (Waldman et al 1995 J. Infec. Dis. 171: 263-72, Hahn et al 2004 J. Virol. 78: 10023-33, Bentz et al 2006 J. Virol. 80: 11539-55).

In various embodiments, the disclosure provides a combination comprising: an antibody or antigen binding fragment thereof comprising the CDR sequences of antibody 7H3, wherein the antibody or fragment binds to and/or inhibits hCMV glycoprotein gB; and an antibody or antigen binding fragment thereof comprising the CDR sequences of antibody 4I22, wherein the antibody or fragment binds to and/or inhibits a 5-member complex consisting of hCMV glycoproteins gH, gL, UL128, UL130 and UL131A.

The sequences of the heavy chains and light chains of several example antibodies to hCMV, each comprising three CDRs on the heavy chain and three CDRs on the light chain have been determined, as shown herein and in U.S. Pat. No. 8,603,480. The position of the CDR amino acids are defined according to the IMGT numbering system Lefranc et al. 2003. IMGT unique numbering for immunoglobulin and T cell receptor variable domains and Ig superfamily V-like domains. Dev Comp Immunol. 27(1):55-77; Lefranc et al. 1997. Unique database numbering system for immunogenetic analysis. Immunology Today, 18:509; Lefranc (1999) The Immunologist, 7:132-136. The sequences of the CDRs, heavy chains, light chains as well as the sequences of the nucleic acid molecules encoding the CDRs, heavy chains, light chains are disclosed in the sequence listing. Table 1 provides the SEQ ID NOs. for the sequences of the six CDRs of the example antibodies of the disclosure. Tables 2 and 3 provide the SEQ ID NOs for the sequences of the heavy and light chains, respectively, of the example antibodies of the disclosure, and Table 4 provides the SEQ ID NOs for the sequences of the nucleic acid molecules encoding the CDRs, heavy chains and light chains of the antibodies.

TABLE 1

| | SEQ ID NOs. for | SEQ ID NOs. for |
|---|---|---|
| | CDRH1, CDRH2, | CDRL1, CDRL2, |
| Antibody | CDRH3 | CDRL3 |
| 15D8 | 188, 189, 190 | 191, 192, 193 |
| 15D8 variant 1 | 188, 204, 205 | 191, 192, 193 |
| 15D8 variant 2 | 188, 189, 210 | 191, 192, 193 |
| 4N10 | 1, 2, 3 | 4, 5, 6 |
| 10F7 | 17, 18, 19 | 20, 21, 22 |
| 10P3 | 33, 34, 35 | 36, 37, 38 |
| 4I22 | 49, 50, 51 | 52, 53, 54 |
| 8L13 | 113, 114, 115 | 116, 117, 118 |
| 2C12 | 65, 66, 67 | 68, 69, 70 |
| 8C15 | 81, 82, 83 | 84, 85, 86 |
| 9I6 | 97, 98, 99 | 100, 101, 102 |
| 7B13 | 129, 130, 131 | 132, 133, 134 |
| 8J16 | 145, 146, 147 | 148, 149, 150 |
| 8I21 | 174, 175, 176 | 177, 149, 178 |
| 7I13 | 113, 161, 162 | 163, 149, 164 |
| 7H3 | 316, 317, 318 | 319, 320, 321 |
| 7H3 variant 1 | 316, 317, 332 | 319, 320, 321 |
| 6B4 | 336, 337, 338 | 339, 340, 341 |
| 5F1 | 278, 279, 280 | 281, 282, 283 |
| 10C6 | 352, 279, 280 | 281, 282, 283 |
| 4H9 | 296, 297, 298 | 299, 300, 301 |
| 4H9 variant 1 | 296, 312, 298 | 299, 300, 301 |
| 11B12 | 232, 233, 234 | 235, 149, 236 |
| 13H11 | 216, 217, 218 | 219, 220, 221 |
| 3G16 | 246, 247, 248 | 249, 250, 251 |
| 2B11 | 360, 279, 280 | 281, 282, 361 |
| 6L3 | 262, 263, 264 | 265, 266, 267 |

TABLE 2

| Antibody | SEQ ID NOs for Heavy Chains |
|---|---|
| 15D8 | 200 |
| 15D8 variant 1 | 208 |
| 15D8 variant 2 | 212 |
| 4N10 | 13 |
| 10F7 | 29 |
| 10P3 | 45 |
| 4I22 | 61 |
| 8L13 | 125 |
| 2C12 | 77 |
| 8C15 | 93 |
| 9I6 | 109 |
| 7B13 | 141 |
| 8J16 | 157 |
| 8I21 | 184 |
| 7I13 | 170 |
| 7H3 | 328 |
| 7H3 variant 1 | 334 |
| 6B4 | 348 |
| 5F1 | 290 |
| 5F1 variant 1 | 294 |
| 10C6 | 357 |
| 4H9 | 308 |
| 4H9 variant 1 | 314 |
| 11B12 | 242 |
| 13H11 | 228 |
| 3G16 | 258 |
| 2B11 | 367 |
| 6L3 | 274 |

TABLE 3

| Antibody | SEQ ID NO for Light Chains |
|---|---|
| 15D8 | 201 |
| 15D8 variant 1 | 201 |
| 15D8 variant 2 | 213 |
| 4N10 | 14 |
| 10F7 | 30 |
| 10P3 | 46 |
| 4I22 | 62 |
| 8L13 | 126 |
| 2C12 | 78 |
| 8C15 | 94 |
| 9I6 | 110 |
| 7B13 | 142 |
| 8J16 | 158 |
| 8I21 | 185 |
| 7I13 | 171 |
| 7H3 | 329 |
| 7H3 variant 1 | 329 |
| 6B4 | 349 |
| 5F1 | 291 |
| 5F1 variant 1 | 291 |
| 10C6 | 291 |
| 4H9 | 309 |
| 4H9 variant 1 | 309 |
| 11B12 | 243 |
| 13H11 | 229 |
| 3G16 | 259 |
| 2B11 | 368 |
| 6L3 | 275 |

TABLE 4

| | SEQ ID NO for Nucleic Acids encoding CDRs, Heavy |
|---|---|
| | Chains, Light Chains and Variants (CDRH1, CDRH2, |
| | CDRH3, CDRL1, CDRL2, CDRL3 and variants; Heavy |
| Antibody | Chain and variants; and Light Chains and variants) |
| 15D8 | 194-199 and 206, 207, 211; 202 and 209, 214; 203 and 215 |
| 4N10 | 7-12; 15; 16 |
| 10F7 | 23-28; 31; 32 |
| 10P3 | 39-44; 47; 48 |
| 4I22 | 55-60; 63; 64 |
| 8L13 | 119-124; 127; 128 |
| 2C12 | 71-76; 79; 80 |
| 8C15 | 87-92; 95; 96 |
| 9I6 | 103-108, 111, 112 |
| 7B13 | 135-140; 143; 144 |
| 8J16 | 151-156; 159; 160 |
| 8I21 | 179-182, 155, 183; 186; 187 |
| 7I13 | 165, 166, 167, 168, 155, 169; 172; 173 |
| 7H3 | 322-327 and 333; 330 and 335; 331 |
| 6B4 | 342-347; 350; 351 |
| 5F1 | 284-289; 292 and 295; 293 |
| 10C6 | 353-355, 287, 288, 356; 358; 359 |
| 4H9 | 302-307 and 313; 310 and 315; 311 |
| 11B12 | 237-240, 155, 241; 244; 245 |
| 13H11 | 222-227; 230; 231 |
| 3G16 | 252-257; 260; 261 |
| 2B11 | 362-364; 287, 365, 366; 369; 370 |
| 6L3 | 268-273; 276; 277 |

Additional information pertaining to these antibodies is provided herein and in U.S. Pat. No. 8,603,480, which is incorporated in its entirety by reference.

As described herein, a large number of combinations of any two of these antibodies can be devised. However, this work shows that, in contrast to many individual antibodies or combinations thereof, the combination of 7H3 and 4I22 was found to have developability and little to no off-target binding, and to block cell-to-cell fusion and syncytia formation mediated by hCMV.

In one embodiment, the disclosure provides a combination of: an antibody or antigen binding fragment thereof comprising the CDR sequences of antibody 7H3, e.g., the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively, wherein the antibody or fragment binds to and/or inhibits hCMV glycoprotein gB; and an antibody or antigen binding fragment thereof comprising the CDR sequences of antibody 4I22, e.g., the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively, wherein the antibody or fragment binds to and/or inhibits a 5-member complex consisting of hCMV glycoproteins gH, gL, UL128, UL130 and UL131A.

In a further embodiment, the disclosure provides a combination of: an antibody or antigen binding fragment thereof comprising sequences that at least 70%, at least 75%, at least 80%, at least 85%, at least 90%, at least 95%, at least 98%, or at least 99% identical to the amino acid sequences of the CDR sequences of antibody 7H3, e.g., the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively, wherein the antibody or fragment binds to and/or inhibits hCMV glycoprotein gB; and an antibody or antigen binding fragment thereof comprising sequences that at least 70%, at least 75%, at least 80%, at least 85%, at least 90%, at least 95%, at least 98%, or at least 99% identical to the amino acid sequences of the CDR sequences of antibody 4I22, e.g., the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively, wherein the antibody or fragment binds to and/or inhibits a 5-member complex consisting of hCMV glycoproteins gH, gL, UL128, UL130 and UL131A.

In a further embodiment, the disclosure provides a combination of: an antibody or antigen binding fragment thereof comprising the sequences of heavy and light chain variable regions of antibody 7H3, e.g., SEQ ID NOs: 328 and 329, respectively, wherein the antibody or fragment binds to and/or inhibits hCMV glycoprotein gB; and an antibody or antigen binding fragment thereof comprising the sequences of heavy and light chain variable regions of antibody 4I22, e.g., SEQ ID NOs: 61 and 62, respectively, wherein the antibody or fragment binds to and/or inhibits a 5-member complex consisting of hCMV glycoproteins gH, gL, UL128, UL130 and UL131A.

In a further embodiment, the disclosure provides a combination of: an antibody or antigen binding fragment thereof comprising sequences that are at least 70%, at least 75%, at least 80%, at least 85%, at least 90%, at least 95%, at least 98%, or at least 99% identical to the sequences of heavy and light chain variable regions of antibody 7H3, e.g., SEQ ID NOs: 328 and 329, respectively, wherein the antibody or fragment binds to and/or inhibits hCMV glycoprotein gB; and an antibody or antigen binding fragment thereof comprising sequences that are at least 70%, at least 75%, at least 80%, at least 85%, at least 90%, at least 95%, at least 98%, or at least 99% identical to the sequences of heavy and light chain variable regions of antibody 4I22, e.g., SEQ ID NOs: 61 and 62, respectively, wherein the antibody or fragment binds to and/or inhibits a 5-member complex consisting of hCMV glycoproteins gH, gL, UL128, UL130 and UL131A.

By “7H3” is also meant any antibody which comprises the CDR sequences of 7H3, as described herein, e.g., the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively.

By “4I22” is meant any antibody which comprises the CDR sequences of 4I22, as described herein, e.g., the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively, or as set forth in Table 1.

The disclosure provides combinations of two or more antibodies or antigen binding fragments, as a non-limiting example, the combination of antibodies and antigen binding fragments comprising the CDR sequences of 7H3 and 4I22. In some embodiments, the first antibody of a combination is 7H3, and the second is 4I22. In some embodiments, the first antibody of a combination is 4I22, and the second is 7H3.

In another aspect, the disclosure also includes nucleic acid sequences encoding part or all of the light and heavy chains and CDRs of the antibodies of the present disclosure. In one embodiment, nucleic acid sequences according to the disclosure include nucleic acid sequences having at least 70%, at least 75%, at least 80%, at least 85%, at least 90%, at least 95%, at least 98%, or at least 99% identity to the nucleic acid encoding a heavy or light chain of an antibody of the disclosure. In another embodiment, a nucleic acid sequence of the disclosure has the sequence of a nucleic acid encoding a heavy or light chain CDR of an antibody of the disclosure. For example, a nucleic acid sequence according to the disclosure comprises a sequence that is at least 75% identical to the nucleic acid sequences of SEQ ID NOs: 322-327 and 333; 330 and 335; 331 (nt sequences encoding 7H3 or the CDRs thereof) and 55-60; 63; 64 (nt sequences encoding 4I22 or CDRs thereof), as listed in Table 4. In one embodiment, the nucleic acid sequence according to the disclosure comprises a sequence that is at least 80%, at least 85%, at least 90%, at least 95%, at least 97%, at least 98%, or at least 99% identical or identical to the nucleic acid sequences of SEQ ID NOs: 322-327 and 333; 330 and 335; 331 (nt sequences encoding 7H3 or the CDRs thereof) and 55-60; 63; 64 (nt sequences encoding 4I22 or CDRs thereof).

Due to the redundancy of the genetic code, variants of these sequences will exist that encode the same amino acid sequences. These variants are included within the scope of the disclosure.

Variant antibodies that neutralize hCMV infection are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code, as mentioned above or may be produced due to errors in transcription or translation.

Further variants of the antibody sequences having improved affinity and/or potency may be obtained using methods known in the art and are included within the scope of the disclosure. For example, amino acid substitutions may be used to obtain antibodies with further improved affinity. Alternatively, codon optimisation of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences of the disclosure are also within the scope of the disclosure.

In one embodiment variant antibody sequences that neutralize hCMV infection may share 70% or more (i.e. 75%, 80%, 85%, 90%, 95%, 97%, 98%, 99% or more) amino acid sequence identity with the sequences recited in the application. In some embodiments such sequence identity is calculated with regard to the full length of the reference sequence (i.e. the sequence recited in the application). In some further embodiments, percentage identity, as referred to herein, is as determined using BLAST version 2.1.3 using the default parameters specified by the NCBI (the National Center for Biotechnology Information) [Blosum 62 matrix; gap open penalty=11 and gap extension penalty=1].

Further included within the scope of the disclosure are vectors, for example expression vectors, comprising a nucleic acid sequence according to the disclosure. Cells transformed with such vectors are also included within the scope of the disclosure. Examples of such cells include but are not limited to, eukaryotic cells, e.g. yeast cells, animal cells or plant cells. In one embodiment the cells are mammalian, e.g. human, CHO, HEK293T, PER.C6, NSO, myeloma or hybridoma cells.

The disclosure also relates to combinations of monoclonal antibodies that bind to an epitope capable of binding the antibodies of the disclosure, including, but not limited to, combinations of any two or more antibodies or antigen binding fragments, including monoclonal antibodies. These include, without limitation, the combination of antibodies and antigen binding fragments comprising the CDR sequences of 7H3 and 4I22.

### Combinations of 7H3 and 4I22

The disclosure provides a combination of: an antibody or antigen binding fragment thereof comprising the CDR sequences of antibody 7H3, e.g., the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively, wherein the antibody or fragment binds to and/or inhibits hCMV glycoprotein gB; and an antibody or antigen binding fragment thereof comprising the CDR sequences of antibody 4I22, e.g., the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively, wherein the antibody or fragment binds to and/or inhibits a 5-member complex consisting of hCMV glycoproteins gH, gL, UL128, UL130 and UL131A; the disclosure also provides various dosages, ratios and minimum trough serum concentrations of these antibodies.

As detailed herein, e.g., Example 3, tests were performed with various antibodies described herein and in U.S. Pat. No. 8,603,480, and various combinations thereof. In various combinations, for example, an antibody from a subgroup of Group 1 was tested in combination with an antibody from a subgroup of Group 2 (as the Groups are defined in Table 6).

Various combinations can be envisioned of antibodies or fragments to hCMV. For example, one antibody may bind to an epitope in the hCMV UL128 protein, an epitope formed by the hCMV proteins UL130 and UL131A, an epitope formed by the hCMV proteins UL128, UL130 and UL131A, an epitope formed by the hCMV proteins gH, gL, UL128 and UL130, an epitope in the hCMV gB protein, an epitope in the hCMV gH protein, or an epitope formed by the hCMV proteins gM and gN, while another may bind to a different epitope in the hCMV UL128 protein, an epitope formed by UL130 and UL131A, an epitope formed by UL128, UL130 and UL131A, an epitope formed by gH, gL, UL128 and UL130, gB, gH, gL, gM, gN, gO, or an epitope formed by gM and gN, and another antibody can bind to a different epitope. Without being bound to any theory, this disclosure suggests that one antibody may be targeted to the mechanism that mediates infection of fibroblasts, while the other antibody may be targeted to the mechanism that mediates infection of endothelial cells. For optimal clinical effect it may well be advantageous to address both mechanisms of hCMV infection and maintenance.

Many individual antibodies and combinations thereof were tested, and the combination of 7H3 and 4I22 was found to have developability and little to no off-target binding, and to have blocked cell-to-cell fusion and syncytia formation mediated by hCMV.

In contrast, many other antibodies were found to have or predicted to have glycosylation sites, deamidation sites, or unlinked cys residues, or to show off-target effects, such as binding to skin antigens.

In various embodiments, the disclosure provides compositions and methods of their use, comprising the combination of antibodies or antigen binding fragments thereof comprising the CDR sequences of antibodies 7H3 and 4I22. In one embodiment, the disclosure provides a composition comprising fully human affinity matured IgG1 monoclonal antibodies or antigen binding fragments thereof comprising the CDR sequences of antibodies 7H3 and 4I22. Antibodies 7H3 and 4I22 were isolated directly from different immortalized B cells and both bind to and inhibit the function of viral glycoproteins essential for hCMV infectivity. 7H3 blocks hCMV glycoprotein B (gB) function while 4I22 blocks the function of the 5-member complex, consisting of hCMV glycoproteins gH, gL, UL128, UL130, and UL131A. The combination of 7H3 and 4I22 neutralizes hCMV infection of all cell types tested by both blocking the initial infection of cells and the subsequent cell to cell spread of virus.

hCMV isolates resistant to either 7H3 or 4I22 can be selected for in vitro after serial passage of virus in the presence of either 7H3 or 4I22 alone. In laboratory experimentation, however, no escape virus had been generated in the presence of both antibodies even after 439 days of continuous culture. Of note, 4I22 can neutralize 7H3-resistant hCMV, and 7H3 can neutralize 4I22-resistant hCMV at antibody concentrations similar to those required to inhibit wild-type virus.

In some embodiments, both 7H3 and 4I22 are fully human IgG1 antibodies with unaltered Fc regions. The neonatal Fc receptor (FcRn) affinities of each antibody were determined to be within expected values, suggesting that the antibodies should bind to FcRn receptors in vivo and, therefore, undergo typical FcRn-mediated disposition with resulting antibody recycling in adults and cross-placental transfer to the fetus during pregnancy. The unaltered Fc of both antibodies also makes effector functions such as antibody-dependent cell-mediated cytotoxicity (ADCC) possible. In vitro, 7H3 and 4I22 are capable of binding to the surface of hCMV-infected cells to mediate ADCC with levels similar or lower than hCMV hyperimmune globulin. However, targeting cells that express hCMV antigens for either antibody-dependent destruction would likely be a benefit of therapy comprising the two antibodies.

7H3 and 4I22 exhibited no off-target binding to human protein microarrays and in good laboratory practice (GLP) human tissue cross-reactivity studies. Scattered binding was noted in human tissues but only occurred to cells confirmed to be positive for hCMV DNA and RNA by in situ hybridization. In earlier non-GLP tissue cross-reactivity studies, no off-target binding was observed in human adult and fetal tissues.

Rats in a 4-week GLP toxicology study received 5 weekly intravenous doses of both antibodies, 7H3 and 4I22, or of placebo. No adverse effects were noted at all doses tested, including at the highest dose administered: 500 mg/kg of 7H3 and 50 mg/kg of 4I22. No evidence of treatment-related immunogenicity to either antibody was noted. The pharmacokinetic (PK) profiles of 7H3 and 4I22 were typical of human IgG1 antibodies, with dose-related increases in exposure, slow clearance, and long terminal elimination half-lives.

Using the combination of antibodies or fragments comprising the CDR sequences of 7H3 and 4I22 has several advantages. (1) Although 7H3 inhibited hCMV infection of all cell types tested, 4I22 is a high affinity and potency neutralizing antibody that targets the 5-member complex, which is required for the infection of cell types likely required for systemic spread of hCMV. (2) Antibodies directed against gB (such as 7H3) and the 5-member complex (such as 4I22) are the predominant neutralizing antibodies detected after a natural infection. Targeting both gB and the 5-member complex will likely maximize viral neutralization and control of hCMV infections in vivo. (3) In vitro data suggest that the combination of 7H3 and 4I22 will significantly decrease the development of viral resistance to either antibody.

The combination of antibodies or fragments comprising the CDR sequences of 7H3 and 4I22 offers the potential to be a safe and well-tolerated alternative to currently available therapies for the prevention and treatment of hCMV disease in pregnant, immunocompromised or immunosuppressed individuals, subjects or patients as well as possibly congenital hCMV in neonates.

### Dosages, Ratios and Minimum Serum Trough Concentrations of a Combination of 7H3 and 4I22

The disclosure provides a combination comprising: a first antibody or fragment comprising the CDR sequences of 7H3 and a second antibody or fragment comprising the CDR sequences of 4I22. In various embodiments, the first antibody or antigen binding fragment thereof is administered at a dosage of about 1 -50, 2.5 to 25, 5 to 20, 5 to 10, about 5 or 5 mg/kg body weight. In various embodiments, the second antibody or antigen binding fragment thereof is administered at a dosage of about 0.1 to 5.0, 0.25 to 2.5, .5 to 2, 0.5 to 1, about 0.5 or 0.5 mg/kg body weight. In various embodiments, the dosages of the first and second antibodies or fragments are 5 and 0.5 mg/kg body weight, respectively. In various embodiments, the ratios of the first antibody or fragment : second antibody or fragment, as administered or as included in a composition, are between about 7.5:1 and about 12.5:1; about 10:1, or 10:1. In some embodiments, the ratio is about 7.5:1. In some embodiments, the ratio is about 12.5:1. In some embodiments, the ratio is about 5:1. In some embodiments, the ratio is about 15:1. In some embodiments, the ratio is about 20:1. In some embodiments, the ratio is about 5:1 to about 20:1. In various embodiments, the dosages of the first and second antibody or fragment and/or dosing frequency are sufficient to sufficient to maintain a minimum trough serum concentration of at least about 7.4 μg/ml and 0.74 μg/ml, respectively, of the first and second antibody or fragment. In various embodiments, the dosages are administered intraperitoneally, orally, subcutaneously, intramuscularly, topically or intravenously. In various embodiments, the dosages of the first and second antibody or antigen binding fragment thereof are administered simultaneously, on the same day, and/or in any order.

Mechanistic PK/pharmacodynamic (PD) modeling, assuming typical human IgG1 PK parameters as well as using in vitro viral binding and neutralization data and in vivo hCMV viral load data from transplant recipients, predicts that a minimum trough serum concentration needs to be maintained for each monoclonal antibody in order to prevent virus rebound.

By “minimum trough serum concentration” or “minimal trough serum concentration” or minimum or minimal “serum trough concentration” or the like is meant the point of minimum concentration of a drug, in this case, either of the two antibodies 7H3 or 4I22, immediately before administering the next dose of the antibody.

In some embodiments, the term “trough serum concentration” refers to the serum drug concentration at a time after delivery of a previous dose and immediately prior to delivery of the next subsequent dose of drug in a series of doses. Generally, the trough serum concentration is a minimum sustained efficacious drug concentration in the series of drug administrations. Also, the trough serum concentration is frequently targeted as a minimum serum concentration for efficacy because it represents the serum concentration at which another dose of drug is to be administered as part of the treatment regimen. If the delivery of drug is by intravenous administration, the trough serum concentration is most preferably attained within a few days or a week or two of a front loading initial drug delivery. According to the disclosure, the trough serum concentration is preferably attained in 4 weeks or less, preferably 3 weeks or less, more preferably 2 weeks or less, most preferably in 1 week or less, including 1 day or less using any of the drug delivery methods disclosed herein.

The model prediction along with the in vitro viral breakthrough data indicate that in order to durably suppress viral replication, minimal trough serum concentrations of 7.4 μg/mL (for 7H3) and 0.74 μg/mL (for 4I22) need to be maintained.

An “efficacious range” of an antibody or antigen-binding fragment thereof is any range which is as high or higher than the minimal trough serum concentration.

In one embodiment, the disclosure provides a method of neutralizing hCMV infection, comprising the steps of: (a) administering one or more doses of a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively; wherein the one or more doses are sufficient to maintain a minimum trough serum concentration of at least about 7.4 μg/ml; and (b) administering one or more doses of a second antibody or antigen binding fragment thereof, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively; wherein the one or more doses are sufficient to maintain a minimum trough serum concentration of at least about 0.74 μg/ml; wherein steps (a) and (b) can be performed simultaneously or in any order.

The model prediction along with the in vitro viral resistance data suggest that intravenous doses of 5 and 0.5 mg/kg given once every 4 weeks for 7H3 and 4I22, respectively, are required to maintain minimum trough serum concentrations that ensure maximum inhibition of viral replication and prevention of viral resistance over prolonged periods of time.

Thus, the antibodies 7H3 and 4I22 and the combination thereof were both found to be effective binders to hCMV glycoproteins with excellent neutralization potency; they showed developability and little to no off-target binding, and blocked cell-to-cell fusion and syncytia formation mediated by hCMV. This combination is particularly efficacious when administered at the dosages, ratios and minimum serum concentrations described herein.

### Additional Uses of the Antibodies

Monoclonal and recombinant antibodies are particularly useful in identification and purification of the individual polypeptides or other antigens against which they are directed. The antibodies of the disclosure have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme. The antibodies may also be used for the molecular identification and characterisation (epitope mapping) of antigens.

Antibodies of the combinations of the disclosure can be coupled to a drug for delivery to a treatment site or coupled to a detectable label to facilitate imaging of a site comprising cells of interest, such as cells infected with hCMV. Methods for coupling antibodies to drugs and detectable labels are well known in the art, as are methods for imaging using detectable labels. Labelled antibodies may be employed in a wide variety of assays, employing a wide variety of labels. Detection of the formation of an antibody-antigen complex between an antibody of the disclosure and an epitope of interest (an hCMV epitope) can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like. See for example, references U.S. Pat. Nos. 3,766,162; 3,791,932; 3,817,837; 4,233,402.

An antibody according to a combination of the disclosure may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. pp. 475-506 (Editrice Kurtis, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, an antibody can be conjugated to a second antibody to form an antibody heteroconjugate as described in U.S. Pat. No. 4,676,980. In addition, linkers may be used between the labels and the antibodies of the disclosure, U.S. Pat. No. 4,831,175. Antibodies or, antigen-binding fragments thereof may be directly labelled with radioactive iodine, indium, yttrium, or other radioactive particle known in the art, U.S. Pat. No. 5,595,721. Treatment may consist of a combination of treatment with conjugated and non-conjugated antibodies administered simultaneously or subsequently WO00/52031; WO00/52473.

Antibodies of a combination of the disclosure may also be attached to a solid support.

Additionally, antibodies of a combination of the disclosure, or functional antibody fragments thereof, can be chemically modified by covalent conjugation to a polymer to, for example, increase their circulating half-life, for example. Examples of polymers, and methods to attach them to peptides, are shown in U.S. Pat. Nos. 4,766,106; 4,179,337; 4,495,285; 4,609,546. In some embodiments the polymers may be selected from polyoxyethylated polyols and polyethylene glycol (PEG). PEG is soluble in water at room temperature and has the general formula: R(O—CH₂—CH₂)ₙ O—R where R can be hydrogen, or a protective group such as an alkyl or alkanol group. In one embodiment the protective group may have between 1 and 8 carbons. In a further embodiment the protective group is methyl. The symbol n is a positive integer. In one embodiment n is between 1 and 1,000. In another embodiment n is between 2 and 500. In one embodiment the PEG has an average molecular weight between 1,000 and 40,000. In a further embodiment the PEG has a molecular weight between 2,000 and 20,000. In yet a further embodiment the PEG has a molecular weight of between 3,000 and 12,000. In one embodiment PEG has at least one hydroxy group. In another embodiment the PEG has a terminal hydroxy group. In yet another embodiment it is the terminal hydroxy group which is activated to react with a free amino group on the inhibitor. However, it will be understood that the type and amount of the reactive groups may be varied to achieve a covalently conjugated PEG/antibody of the present disclosure.

Water-soluble polyoxyethylated polyols are also useful in the present disclosure. They include polyoxyethylated sorbitol, polyoxyethylated glucose, polyoxyethylated glycerol (POG), and the like. In one embodiment, POG is used. Without being bound by any theory, this disclosure suggests that, because the glycerol backbone of polyoxyethylated glycerol is the same backbone occurring naturally in, for example, animals and humans in mono-, di-, triglycerides, this branching would not necessarily be seen as a foreign agent in the body. In some embodiments POG has a molecular weight in the same range as PEG The structure for POG is shown in Knauf et al. (1988) J. Bio. Chem. 263:15064-15070, and a discussion of POG/IL-2 conjugates is found in U.S. Pat. No. 4,766,106.

Another drug delivery system that can be used for increasing circulatory half-life is the liposome. Methods of preparing liposome delivery systems are discussed in Gabizon et al. (1982) Cancer Research 42:4734; Cafiso (1981) Biochem. Biophys. Acta 649:129; and Szoka (1980) Ann. Rev. Biophys. Eng. 9:467. Other drug delivery systems are known in the art and are described in, for example, Poznansky et al. (1980) Drug Delivery Systems (R. L. Juliano, ed., Oxford, N.Y.) pp. 253-315; and Poznansky (1984) Pharm Revs 36:277.

Antibodies of the disclosure may be provided in purified form. Typically, the antibody will be present in a composition that is substantially free of other polypeptides e.g. where less than 90% (by weight), usually less than 60% and more usually less than 50% of the composition is made up of other polypeptides.

Antibodies of the disclosure may be immunogenic in non-human (or heterologous) hosts e.g. in mice. In particular, the antibodies may have an idiotope that is immunogenic in non-human hosts, but not in a human host. Antibodies of the disclosure for human use include those that cannot be easily isolated from hosts such as mice, goats, rabbits, rats, non-primate mammals, etc. and cannot generally be obtained by humanisation or from xeno-mice.

Antibodies of the disclosure can be of any isotype (e.g. IgA, IgG, IgM i.e. an α, γ or μheavy chain), but will generally be IgG. Within the IgG isotype, antibodies may be IgG1, IgG2, IgG3 or IgG4 subclass. Antibodies of the disclosure may have a κ or a λ light chain.

### Production of Antibodies

Monoclonal antibodies according to the disclosure can be made by any method known in the art. The general methodology for making monoclonal antibodies using hybridoma technology is well known Kohler, G. and Milstein, C., 1975, Nature 256:495-497; Kozbar et al. 1983, Immunology Today 4:72.Preferably, the alternative EBV immortalisation method described in WO2004/076677 is used.

Using the method described in WO2004/076677, B cells producing the antibody of the disclosure can be transformed with EBV in the presence of a polyclonal B cell activator. Transformation with EBV is a standard technique and can easily be adapted to include polyclonal B cell activators.

Additional stimulants of cellular growth and differentiation may optionally be added during the transformation step to further enhance the efficiency. These stimulants may be cytokines such as IL-2 and IL-15. In one aspect, IL-2 is added during the immortalisation step to further improve the efficiency of immortalisation, but its use is not essential.

The immortalised B cells produced using these methods can then be cultured using methods known in the art and antibodies isolated therefrom.

The antibodies of the disclosure can also be made by culturing single plasma cells in microwell culture plates using the method described in UK Patent Application 0819376.5. Further, from single plasma cell cultures, RNA can be extracted and single cell PCR can be performed using methods known in the art. The VH and VL regions of the antibodies can be amplified by RT-PCR, sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

Monoclonal antibodies may be further purified, if desired, using filtration, centrifugation and various chromatographic methods such as HPLC or affinity chromatography. Techniques for purification of monoclonal antibodies, including techniques for producing pharmaceutical-grade antibodies, are well known in the art.

Fragments of the monoclonal antibodies of the disclosure can be obtained from the monoclonal antibodies by methods that include digestion with enzymes, such as pepsin or papain, and/or by cleavage of disulfide bonds by chemical reduction. Alternatively, fragments of the monoclonal antibodies can be obtained by cloning and expression of part of the sequences of the heavy or light chains. Antibody “fragments” may include Fab, Fab’, F(ab')₂ and Fv fragments. The disclosure also encompasses single-chain Fv fragments (scFv) derived from the heavy and light chains of a monoclonal antibody of the disclosure e.g. the disclosure includes a scFv comprising the CDRs from an antibody of the disclosure. Also included are heavy or light chain monomers and dimers as well as single chain antibodies, e.g. single chain Fv in which the heavy and light chain variable domains are joined by a peptide linker.

Standard techniques of molecular biology may be used to prepare DNA sequences coding for the antibodies or fragments of the antibodies of the present disclosure. Desired DNA sequences may be synthesised completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present disclosure or fragments thereof Bacterial, for example E. coli, and other microbial systems may be used, in part, for expression of antibody fragments such as Fab and F(ab′)₂ fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, HEK293T, PER.C6, NS0, myeloma or hybridoma cells.

The present disclosure also provides a process for the production of an antibody molecule according to the present disclosure comprising culturing a host cell comprising a vector of the present disclosure under conditions suitable for leading to expression of protein from DNA encoding the antibody molecule of the present disclosure, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Alternatively, antibodies according to the disclosure may be produced by i) expressing a nucleic acid sequence according to the disclosure in a cell, and ii) isolating the expressed antibody product. Additionally, the method may include iii) purifying the antibody.

### Screening and Isolation of B Cells

Transformed B cells may be screened for those producing antibodies of the desired antigen specificity, and individual B cell clones may then be produced from the positive cells.

The screening step may be carried out by ELISA, by staining of tissues or cells (including transfected cells), a neutralisation assay or one of a number of other methods known in the art for identifying desired antigen specificity. The assay may select on the basis of simple antigen recognition, or may select on the additional basis of a desired function e.g. to select neutralizing antibodies rather than just antigen-binding antibodies, to select antibodies that can change characteristics of targeted cells, such as their signalling cascades, their shape, their growth rate, their capability of influencing other cells, their response to the influence by other cells or by other reagents or by a change in conditions, their differentiation status, etc.

The cloning step for separating individual clones from the mixture of positive cells may be carried out using limiting dilution, micromanipulation, single cell deposition by cell sorting or another method known in the art.

The immortalised B cell clones of the disclosure can be used in various ways e.g. as a source of monoclonal antibodies, as a source of nucleic acid (DNA or mRNA) encoding a monoclonal antibody of interest, for research, etc.

The disclosure provides a composition comprising immortalised B memory cells, wherein the cells produce antibodies with high neutralizing potency specific for hCMV, and wherein the antibodies are produced at >5pg per cell per day. The disclosure also provides a composition comprising clones of an immortalised B memory cell, wherein the clones produce a monoclonal antibody with a high affinity specific for hCMV, and wherein the antibody is produced at >5pg per cell per day. Preferably said clones produce a monoclonal antibody with a high potency in neutralizing hCMV infection.

### Pharmaceutical Compositions

The disclosure provides a pharmaceutical composition comprising a combination of antibodies or fragments thereof having the CDR sequences of 7H3 and 4I22. A pharmaceutical composition may also contain a pharmaceutically acceptable carrier to allow administration. The carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject or patient.

Within the scope of the disclosure, forms of administration may include those forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the disclosure can be administered directly to the subject. In one embodiment the compositions are adapted for administration to human subjects.

The pharmaceutical compositions of this disclosure may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the disclosure. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g. whether a pharmaceutical should be delivered daily, weekly, monthly, etc. Frequency and dosage may also depend on the severity of symptoms.

Compositions of the disclosure may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition, like Synagis™ (an antibody against an epitope in the A antigenic site of the F protein of RSV) and anti-Her2 antibody Herceptin™, for reconstitution with sterile water containing a preservative). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a subject or patient. For example, a lyophilised antibody can be provided in kit form with sterile water or a sterile buffer.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the disclosure generally have a pH between 5.5 and 8.5, in some embodiments this may be between 6 and 8, and in further embodiments about 7. The pH may be maintained by the use of a buffer. The composition may be sterile and/or pyrogen free. The composition may be isotonic with respect to humans. In one embodiment pharmaceutical compositions of the disclosure are supplied in hermetically-sealed containers.

Pharmaceutical compositions will include an effective amount of one or more antibodies of the disclosure and/or one or more immortalised B cells of the disclosure and/or a polypeptide comprising an epitope that binds an antibody of the disclosure i.e. an amount that is sufficient to treat, ameliorate, or prevent a desired disease or condition, or to exhibit a detectable therapeutic effect. Therapeutic effects also include reduction in physical symptoms. The precise effective amount for any particular subject will depend upon their size and health, the nature and extent of the condition, and the therapeutics or combination of therapeutics selected for administration. The effective amount for a given situation is determined by routine experimentation and is within the judgment of a clinician. For purposes of the present disclosure, an effective dose will generally be from about 0.01 mg/kg to about 50 mg/kg, or about 0.05 mg/kg to about 10 mg/kg of the compositions of the present disclosure in the individual to which it is administered. Known antibody-based pharmaceuticals provide guidance in this respect, e.g., Herceptin™ (an anti-Her2 antibody) is administered by intravenous infusion of a 21 mg/ml solution, with an initial loading dose of 4 mg/kg body weight and a weekly maintenance dose of 2 mg/kg body weight; Rituxan™ (an antibody to CD20) is administered weekly at 375 mg/m²; etc.

In one embodiment compositions can include more than one (e.g. 2, 3, 4, 5, etc.) antibody of the disclosure to provide an additive or synergistic therapeutic effect. In a further embodiment the composition may comprise one or more (e.g. 2, 3, 4, 5, etc.) antibody of the disclosure and one or more (e.g. 2, 3, 4, 5, etc.) additional antibodies that neutralize hCMV infection. The disclosure also comprises combinations of any two or more antibodies or antigen binding fragments. These include, without limitation, the combination of antibodies and antigen binding fragments comprising the CDR sequences of 7H3 and 4I22, further comprising an additional antibody or fragment to hCMV.

In various embodiments, the disclosure provides pharmaceutical compositions comprising 7H3 and/or 4122; and a pharmaceutically acceptable carrier.

Pharmaceutical compositions comprising 7H3 and/or 4I22 can be prepared by any method known in the art. Non-limiting examples are provided here.

In various embodiments, 7H3 150 mg concentrate solution for infusion is a clear to opalescent colorless to yellowish aqueous solution packaged in a 6 mL glass vial with a grey rubber stopper, which is sealed with an aluminum cap with plastic flip-off disk. The vial is overfilled by 20% to allow for the complete removal of the maximum dose (150 mg). 7H3 150 mg concentrate solution for infusion contains, in addition to 7H3 drug substance, L-histidine, L-histidine hydrochloride monohydrate, hydrochloric acid, sucrose and polysorbate 20. In various embodiments, the formulation does not contain any preservative; it is to be used for single-dose administration only. 7H3 150 mg concentrate solution for infusion is suitable for the preparation of infusion solutions for intravenous administration using 50 mL infusion syringes with doses ranging from 40 mg to 6000 mg. In various embodiments, to obtain the desired total volume for infusion, 7H3 concentrate solution can be diluted with the appropriate volume of 5% dextrose, depending on the intended dose, in accordance with the current version of the instructions for compounding and administration.

In various embodiments, 4I22 50 mg powder for solution for infusion is a white to off-white solid lyophilisate packaged in a 2 mL glass vial with grey rubber stopper, which is sealed with an aluminum cap with plastic flip-off disk. The vial is overfilled by 25% to allow for the complete removal of the maximum dose (50 mg). 4I22 50 mg powder for solution for infusion contains, in addition to 4I22 drug substance, L-histidine, hydrochloric acid, sucrose and polysorbate 20. Reconstitution with 1.2 mL water for injection gives an infusion solution with a concentration of 50 mg/mL 4I22. In various embodiments, the formulation does not contain any preservative; it is to be used for single-dose administration only.

In various embodiments, following reconstitution, the 4I22 concentrate solution for infusion is suitable for the preparation of infusion solutions for intravenous administration using 50 mL infusion syringes with doses ranging from 4 mg to 600 mg. In various embodiments, to obtain the desired total volume for infusion, 4I22 concentrate solution for solution can be diluted with the appropriate volume of 5% dextrose, depending on the intended dose, in accordance with the current version of the instructions for compounding and administration.

In various embodiments of the disclosure, various compositions can comprise a first antibody or antigen binding fragment thereof comprising the CDR sequences of 7H3, or a second antibody or antigen binding fragment thereof comprising the CDR sequences of 4I22; these compositions can be mixed together and administered together. Alternatively, the compositions can be kept separate and administered separately.

In various methods described herein, the method comprises the step (e) of administering to a patient or subject: a dose of a first antibody or antigen binding fragment thereof comprising the CDR sequences of 7H3 and a dose of a second antibody or antigen binding fragment thereof comprising the CDR sequences of 4I22. In various embodiments, the doses can be mixed together; e.g., the first and second antibody or fragment can be combined in one composition which is administered. In various other embodiments, the doses can be separated; e.g., the first and second antibody or fragment can be administered as separate compositions.

Antibodies of the disclosure may be administered (either combined or separately) with other therapeutics e.g. with chemotherapeutic compounds, with radiotherapy, etc. Preferred therapeutic compounds include anti-viral compounds such as ganciclovir, foscarnet and cidofovir. Such combination therapy provides an additive or synergistic improvement in therapeutic efficacy relative to the individual therapeutic agents when administered alone. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Antibodies may be administered to those subjects or patients who have previously shown no response to treatment for hCMV infection, i.e. have been shown to be refractive to anti-hCMV treatment. Such treatment may include previous treatment with an anti-viral agent. This may be due to, for example, infection with an anti-viral resistant strain of hCMV.

In compositions of the disclosure that include antibodies of the disclosure, the antibodies may make up at least 50% by weight (e.g. 60%, 70%, 75%, 80%, 85%, 90%, 95%, 97%, 98%, 99% or more) of the total protein in the composition. The antibodies are thus in purified form.

The disclosure provides a method of preparing a pharmaceutical, comprising the steps of: (i) preparing an antibody of the disclosure; and (ii) admixing the purified antibody with one or more pharmaceutically-acceptable carriers.

The disclosure also provides a method of preparing a pharmaceutical, comprising the step of admixing an antibody with one or more pharmaceutically-acceptable carriers, wherein the antibody is a monoclonal antibody that was obtained from a transformed B cell of the disclosure. Thus the procedures for first obtaining the monoclonal antibody and then preparing the pharmaceutical can be performed at very different times by different people in different places (e.g. in different countries).

As an alternative to delivering antibodies or B cells for therapeutic purposes, it is possible to deliver nucleic acid (typically DNA) that encodes the monoclonal antibody (or active fragment thereof) of interest to a subject, such that the nucleic acid can be expressed in the subject in situ to provide a desired therapeutic effect. Suitable gene therapy and nucleic acid delivery vectors are known in the art.

Compositions may include an antimicrobial, particularly if packaged in a multiple dose format. They may comprise a detergent e.g., a Tween (polysorbate), such as Tween 80. Detergents are generally present at low levels e.g. <0.01%. Compositions may also include sodium salts (e.g. sodium chloride) to give tonicity. A concentration of 10+2mg/ml NaCl is typical.

Compositions may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material.

The pH of a composition for lyophilisation may be adjusted to around 6.1 prior to lyophilisation.

The compositions of the disclosure may also comprise one or more immunoregulatory agents. In one embodiment, one or more of the immunoregulatory agents include(s) an adjuvant.

The epitope compositions of the disclosure may elicit both a cell mediated immune response as well as a humoral immune response in order to effectively address a hCMV infection. This immune response may induce long lasting (e.g. neutralizing) antibodies and a cell mediated immunity that can quickly respond upon exposure to hCMV.

hCMV Disease

Human cytomegalovirus (hCMV) infection is common, with 30 to 100% of the population worldwide infected (Ho 2008). Most infections are asymptomatic or mild but significant complications can occur in immunocompromised individuals. These include hematopoietic stem cell and solid organ transplant recipients, individuals infected with the human immunodeficiency virus (HIV), and neonates exposed to hCMV in utero. Because hCMV establishes a persistent latent infection after an initial infection, disease is not limited to individuals acutely infected (Fishman and Rubin 1998). All individuals previously infected are at risk for reactivation of hCMV replication and, if immunocompromised, significant disease. In addition, because hCMV can infect a wide variety of different cell types, hCMV disease can affect almost any organ (Ljungman et al 2010).

Among transplant recipients, hCMV disease and complications associated with active hCMV infection are significant causes of morbidity and mortality. Pneumonia is the most serious manifestation of hCMV among recipients of hematopoietic stem cell transplants, with mortality often exceeding 50% (Ljungman et al 2010). Other hCMV manifestations after stem cell transplantation include gastroenteritis, hepatitis, retinitis and encephalitis (Boeckh and Ljungman 2009). In addition, active hCMV infection is a risk factor for acute and chronic graft-versus-host disease. Approximately 80% of stem cell recipients will develop an active hCMV infection after transplantation if no prophylaxis is given, and 20 to 35% will develop hCMV disease (Ljungman et al 2010).

Because of the morbidity and mortality associated with hCMV disease, most clinicians use strategies to prevent hCMV disease in transplant recipients (Torres-Madriz and Boucher 2008; Boeckh and Ljungman 2009). In terms of antiviral agents, prevention can be achieved by prophylaxis, in which therapy is given during the period of highest risk to prevent hCMV replication (as measured by viral load), or by preemptive therapy, in which therapy is initiated after hCMV replication is detected (viral load above a given value) but before disease develops. In general, prophylaxis is associated with less hCMV-related sequelae but more drug toxicity than preemptive therapy.

hCMV hyperimmune globulin can be used to prevent hCMV infection and disease in select solid organ transplant recipients (Snydman et al 1987; Snydman 1990), although lower efficacy compared with ganciclovir or valganciclovir limits its use to select high-risk situations (Torres-Madriz and Boucher 2008). Among hematopoietic stem cell transplant recipients, the use of hCMV hyperimmune globulin to prevent hCMV disease is not recommended because efficacy is limited and its use has been associated with veno-occlusive disease of the liver (Boeckh and Ljungman 2009). It is speculated that veno-occlusive disease may be related to hyperviscosity associated with high dose immunoglobulin therapy (Cordonnier et al 2003; Raanani et al 2009). However, veno-occlusive disease was not reported as an outcome in most published trials testing the safety and efficacy of hCMV hyperimmune globulin, and reporting bias cannot be excluded.

A retrospective analysis of two randomized clinical trials comparing high dose immunoglobulin (pooled N =318) with placebo (pooled N =315) found no difference in the incidence or severity of veno-occlusive disease in bone marrow transplant recipients (Sullivan et al 1998). Immunoglobulin or hCMV hyperimmune globulin is often added to ganciclovir or foscarnet when treating hCMV pneumonia (Boeckh and Ljungman 2009).

Although antibodies directed against gB correlate with neutralizing activity (Marshall et al 1992), there is evidence that the major neutralizing antibody response of hCMV hyperimmune globulin is directed against the 5-member complex (Wang et al 2011; Fouts et al 2012). However, such antibodies cannot block the infection of fibroblasts, which requires that the hCMV express the 3-member complex but not the 5-member complex (Wang and Shenk 2005). Thus, a combination of anti-gB (7H3) and anti-5-member complex (4I22) antibodies that can inhibit infection of fibroblasts as well as endothelial and hematopoietic cells should be able to block replication as well as systemic spread of hCMV.

### Medical Treatments and Uses

The antibodies, antibody fragments of the disclosure or derivatives and variants thereof and combinations thereof may be used for the treatment of hCMV infection, for the prevention of hCMV infection or for the diagnosis of hCMV infection.

Methods of diagnosis may include contacting an antibody or an antibody fragment with a sample. Such samples may be tissue samples taken from, for example, salivary glands, lung, liver, pancreas, kidney, ear, eye, placenta, alimentary tract, heart, ovaries, pituitary, adrenals, thyroid, brain or skin. The methods of diagnosis may also include the detection of an antigen/antibody complex.

The disclosure therefore provides (i) an antibody, an antibody fragment, or variants and derivatives thereof and combinations thereof according to the disclosure, (ii) an immortalised B cell clone according to the disclosure, (iii) an epitope capable of binding an antibody of the disclosure or (iv) a ligand, preferably an antibody, capable of binding an epitope that binds an antibody of the disclosure for use in therapy.

Also provided is a method of treating a subject or patient comprising administering to that subject or patient (i) an antibody, an antibody fragment, or variants and derivatives thereof and combinations thereof according to the disclosure, or, a ligand, preferably an antibody, capable of binding an epitope that binds an antibody of the disclosure.

The disclosure also provides the use of (i) an antibody, an antibody fragment, or variants and derivatives thereof and combinations thereof according to the disclosure, (ii) an immortalised B cell clone according to the disclosure, (iii) an epitope capable of binding an antibody of the disclosure, or (iv) a ligand, preferably an antibody, that binds to an epitope capable of binding an antibody of the disclosure, in the manufacture of a medicament for the prevention or treatment of hCMV infection.

The disclosure provides a composition for use as a medicament for the prevention or treatment of an hCMV infection. It also provides the use of an antibody and/or a protein comprising an epitope or combinations thereof to which such an antibody binds in the manufacture of a medicament for treatment of a subject or patient and/or diagnosis in a subject or patient. It also provides a method for treating a subject in need of treatment, comprising the step (e) of administering a composition of the disclosure to the subject. In some embodiments the subject may be a human. One way of checking efficacy of therapeutic treatment involves monitoring disease symptoms after administration of the composition of the disclosure. Treatment can be a single dose schedule or a multiple dose schedule.

In one embodiment, an antibody of the disclosure, an antigen-binding fragment thereof, an epitope or a composition of the disclosure is administered to a subject in need of such prophylactic or therapeutic treatment. Such a subject includes, but is not limited to, one who is particularly at risk of, or susceptible to, hCMV infection. Example subjects include, but are not limited to, immunocompromised subjects or hCMV-seronegative or hCMV recently infected pregnant women. Example immunocompromised subjects include, but are not limited to, those afflicted with HIV or those undergoing immunosuppressive therapy.

Antibodies of the disclosure and antigen-binding fragments thereof or combinations thereof can also be used in passive immunisation. Further, as described in the present disclosure, they may also be used in a kit for the diagnosis of hCMV infection.

In various embodiments, the subject or patient may be pregnant, immunocompromised or immunosuppressed.

Antibodies, antibody fragment, or variants and derivatives thereof or combinations thereof, as described in the present disclosure may also be used in a kit for monitoring vaccine manufacture with the desired immunogenicity.

The disclosure also provides a method of preparing a pharmaceutical, comprising the step of admixing a monoclonal antibody or combinations of antibodies with one or more pharmaceutically-acceptable carriers, wherein the monoclonal antibody is a monoclonal antibody that was obtained from an expression host of the disclosure. Thus the procedures for first obtaining the monoclonal antibody (e.g. expressing it and/or purifying it) and then admixing it with the pharmaceutical carrier(s) can be performed at very different times by different people in different places (e.g. in different countries).

Starting with a transformed B cell of the disclosure, various steps of culturing, sub-culturing, cloning, sub-cloning, sequencing, nucleic acid preparation etc. can be performed in order to perpetuate the antibody expressed by the transformed B cell, with optional optimisation at each step. In a preferred embodiment, the above methods further comprise techniques of optimisation (e.g. affinity maturation or optimisation) applied to the nucleic acids encoding the antibody. The disclosure encompasses all cells, nucleic acids, vectors, sequences, antibodies etc. used and prepared during such steps.

In all these methods, the nucleic acid used in the expression host may be manipulated to insert, delete or amend certain nucleic acid sequences. Changes from such manipulation include, but are not limited to, changes to introduce restriction sites, to amend codon usage, to add or optimise transcription and/or translation regulatory sequences, etc. It is also possible to change the nucleic acid to alter the encoded amino acids. For example, it may be useful to introduce one or more (e.g. 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g. labels) or can introduce tags (e.g. for purification purposes). Mutations can be introduced in specific sites or can be introduced at random, followed by selection (e.g. molecular evolution). For instance, one or more nucleic acids encoding any of the CDR regions, heavy chain variable regions or light chain variable regions of antibodies of the disclosure can be randomly or directionally mutated to introduce different properties in the encoded amino acids. Such changes can be the result of an iterative process wherein initial changes are retained and new changes at other nucleotide positions are introduced. Moreover, changes achieved in independent steps may be combined. Different properties introduced into the encoded amino acids may include, but are not limited to, enhanced affinity.

### General

The word “substantially” does not exclude “completely” e.g. a composition which is “substantially free” from Y may be completely free from Y. Where necessary, the word “substantially” may be omitted from the definition of the disclosure.

The term “about” in relation to a numerical value x means, for example, x+10%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention and prophylaxis. The terms “individual”, “subject” and “patient” mean all mammals including humans. Examples of patients include humans, cows, dogs, cats, horses, goats, sheep, pigs, and rabbits. Generally, the subject or patient is a human.

### EXAMPLES

Example embodiments of the present disclosure are provided in the following examples. The following examples are presented only by way of illustration and to assist one of ordinary skill in using the disclosure. The examples are not intended in any way to otherwise limit the scope of the disclosure.

### Example 1

### Cloning of B Cells and Screening for hCMV Neutralizing Activity

Donors with high hCMV neutralizing antibody titres in the serum were identified. Memory B cells were isolated and immortalised using EBV and CpG as described in WO2004/076677. Briefly, memory B cells were isolated by negative selection using CD22 beads, followed by removal of IgM⁺, IgD⁺, IgA⁺ B cells using specific antibodies and cell sorting. The sorted cells (IgG⁺) were immortalized with EBV in the presence of CpG 2006 and irradiated allogeneic mononuclear cells. Replicate cultures each containing 50 memory B cells were set up in twenty 96 well U bottom plates. After two weeks the culture supernatants were collected and tested for their capacity to neutralize hCMV infection of either fibroblasts or epithelial cells in separate assays. B cell clones were isolated from positive polyclonal cultures as described in WO2004/076677. IgG concentrations in the supernatant of selected clones were determined using an IgG-specific ELISA.

For the viral neutralization assay a titrated amount of a clinical hCMV isolate was mixed with an equal volume of culture supernatant or with dilutions of human sera containing neutralizing antibodies. After 1 hour incubation at room temperature the mixture was added to confluent monolayers of either endothelial cells (e.g. HUVEC cells or HMEC-1 cells), epithelial cells (e.g. ARPE retinal cells), fibroblasts (e.g. MRC-9 or mesenchymal stromal cells) or myeloid cells (e.g. monocyte-derived dendritic cells) in 96 well flat-bottom plates and incubated at 37° C. for two days. The supernatant was discarded, the cells were fixed with cold methanol and stained with a mixture of mouse monoclonal antibodies to hCMV early antigens, followed by a fluorescein-labeled goat anti mouse Ig. The plates were analyzed using a fluorescence microscope. In the absence of neutralizing antibodies the infected cells were 100-1,000/field, while in the presence of saturating concentrations of neutralizing antibodies the infection was completely inhibited. The neutralizing titer is indicated as the concentration of antibody (μg/m1) that gives a 50% or 90% reduction of hCMV infection.

Table 5A shows the neutralization of a hCMV clinical isolate (VR1814) on both a fibroblastic cell line (MRC-9) and a human retinal epithelial cell line (ARPE). Some antibodies neutralized hCMV infection of epithelial cells (ARPE) but they did not neutralize infection of fibroblasts (MRC-9). This agrees with previous data that different proteins are responsible for tropism towards a particular cell type. Most of these antibodies, which are specific for one or more proteins of the gH/gL/UL128/UL130/UL131A protein complex, neutralized hCMV infection of epithelial cells at very low concentrations (50% reduction of hCMV infection at concentrations ranging from 0.01 μg/ml and 0.001 μg/ml). Other antibodies, which are specific for the hCMV protein gB, gH or a combination of gM and gN, neutralized hCMV infection of fibroblasts and epithelial cells with comparable potency. These results show that some of the hCMV neutralizing antibodies are equally potent on both fibroblasts and epithelial cells, while others show differential activity on the two cell types.

Based on the analysis shown in Table 5A, antibodies were grouped into Group 1 (neutralizing hCMV infection of both fibroblasts and epithelial cells) and Group 2 (neutralizing hCMV infection of epithelial cells). Table 5B shows an independent experiment performed using purified antibodies. The results show that Group 2 antibodies neutralized infection of epithelial cells with IC90 values (i.e. the concentration of antibody required to give 90% reduction of viral infection) ranging from 0.007 μg/ml to 0.003 μg/ml while Group 1 antibodies neutralized infection of both fibroblasts and epithelial cells with IC90 values ranging from 0.1 μg/ml to 30 μg/ml. Group 2 antibodies also neutralized infection of endothelial cells (HUVEC) and myeloid cells (monocyte-derived dendritic cells) (data not shown). Group 1 antibodies also neutralized infection of endothelial cells (HUVEC), myeloid cells (monocyte-derived dendritic cells) and bone marrow mesenchymal stromal cells, as shown for some representative antibodies in Table 5C. Antibodies of the disclosure also neutralized infection of endothelial cells (HUVEC) by different hCMV clinical isolates: VR6952 (from urine), VR3480B1 (from blood, ganciclovir-resistant) and VR4760 (from blood, ganciclovir and foscarnet-resistant) (data not shown).

It is anticipated that antibodies that neutralize infection of different cell types may be combined to bring about an additive or synergistic neutralization effect when the different cell types are present during infection. As one example, a neutralizing antibody, such as 15D8 which is potent in neutralizing infection of epithelial cells but does not neutralize infection of fibroblasts might be combined with 3G16 which does have virus neutralizing activity on fibroblasts. As another example, a neutralizing antibody, such as 9I6 which is potent in neutralizing infection of epithelial cells but does not neutralize infection of fibroblasts, might be combined with 6B4 which does have virus neutralizing activity on fibroblasts.

TABLE 5A

| | | | 50% | 50% |
|---|---|---|---|---|
| | | | Neutrali- | Neutrali- |
| | | | zation⁽¹⁾ | zation⁽¹⁾ |
| mAb | Donor | Specificity⁽²⁾ | MRC-9 | ARPE |
| 15D8 | GRA | UL128 | − | ++++ |
| 4N10 | GIO | UL130/UL131A | + | ++++ |
| 10F7 | PAP | UL130/UL131A | + | +++ |
| 10P3 | PEL | UL130/UL131A | − | ++++ |
| 4I22 | PEL | UL130/UL131A | − | +++ |
| 8L13 | PEL | UL130/UL131A | − | +++ |
| 2C12 | PAP | UL128/UL130/UL131A | + | +++ |
| 7B13 | PAP | UL128/UL130/UL131A | − | ++++ |
| 7I13 | PAP | UL128/UL130/UL131A | − | +++ |
| 8C15 | PAP | UL128/UL130/UL131A | − | ++++ |
| 8J16 | PAP | UL128/UL130/UL131A | − | ++++ |
| 9I6 | PEL | UL128/UL130/UL131A | − | ++++ |
| 8I21 | PEL | gH/gL/UL128/UL130 | − | +++ |
| 11B12 | PAP | gH | + | + |
| 13H11 | GRA | gH | + | +++ |
| 3G16 | PEL | gH | + | + |
| 7H3 | PEL | gB | + | − |
| 10C6 | PEL | gB | + | + |
| 5F1 | PEL | gB | + | + |
| 6B4 | PEL | gB | + | + |
| 4H9 | PEL | gB | + | + |
| 6L3 | PEL | gM/gN | Not done | + |

⁽¹⁾Values indicating the concentration of antibody required to give a 50% reduction of hCMV infection of fibroblasts (e.g. MRC-9) or epithelial cells (e.g. ARPE retinal cells). Concentration as follows: ++++ < 0.001 μg/ml; +++ < 0.01 μg/ml; ++ < 0.1 μg/ml; + ≦ 2 μg/ml; − Not neutralizing at the highest concentration tested (2 μg/ml).
⁽²⁾Specificity as defined in Table 6.

TABLE 5B

| | | | | 90% | 90% |
|---|---|---|---|---|---|
| | | | | Neutrali- | Neutrali- |
| | | | | zation⁽¹⁾ | zation⁽¹⁾ |
| Group | mAb | Donor | Specificity⁽²⁾ | MRC-9 | ARPE |
| 2 | 15D8 | GRA | UL128 | nn⁽³⁾ | 0.008 |
| 2 | 4N10 | GIO | UL130/UL131A | nn | 0.02 |
| 2 | 10F7 | PAP | UL130/UL131A | nn | 0.002 |
| 2 | 10P3 | PEL | UL130/UL131A | nn | 0.0025 |
| 2 | 4I22 | PEL | UL130/UL131A | nn | 0.0015 |
| 2 | 8L13 | PEL | UL130/UL131A | nn | 0.001 |
| 2 | 2C12 | PAP | UL128/UL130/ | nn | 0.006 |
| | | | UL131A | | |
| 2 | 7B13 | PAP | UL128/UL130/ | nn | 0.003 |
| | | | UL131A | | |
| 2 | 7I13 | PAP | UL128/UL130/ | nn | 0.008 |
| | | | UL131A | | |
| 2 | 8C15 | PAP | UL128/UL130/ | nn | 0.0025 |
| | | | UL131A | | |
| 2 | 8J16 | PAP | UL128/UL130/ | nn | 0.0008 |
| | | | UL131A | | |
| 2 | 9I6 | PEL | UL128/UL130/ | nn | 0.0007 |
| | | | UL131A | | |
| 2 | 8I21 | PEL | gH/gL/UL128/ | nn | 0.03 |
| | | | UL130 | | |
| 1 | 11B12 | PAP | gH | 3.5 | 1.2 |
| 1 | 13H11 | GRA | gH | 1.12 | 0.4 |
| 1 | 3G16 | PEL | gH | 1.0 | 0.3 |
| 1 | 7H3 | PEL | gB | 3 | 0.6 |
| 1 | 10C6 | PEL | gB | 0.75 | 0.2 |
| 1 | 5F1 | PEL | gB | 0.5 | 0.1 |
| 1 | 6B4 | PEL | gB | 1.0 | 0.15 |
| 1 | 4H9 | PEL | gB | 10 | 0.4 |
| 1 | 2B11 | PEL | gB | 0.75 | 0.2 |
| 1 | 6L3 | PEL | gM/gN | 30 | 10 |

⁽¹⁾Values indicating the concentration of antibody in μg/ml required to give a 90% reduction of hCMV (VR1814) infection of fibroblasts (e.g. MRC-9) or epithelial cells (e.g. ARPE retinal cells).
⁽²⁾Specificity as defined in Table 6.
⁽³⁾nn, not neutralizing at the highest concentration tested (10 μg/ml).

TABLE 5C

| | | | 50% Neutralization⁽¹⁾ | | |
|---|---|---|---|---|---|
| Group | mAb | Specificity | HUVEC | Mo-DC | BM-MSC |
| 1 | 7H3 | gH | nd | 0.06 | 2 |
| 1 | 10C6 | gH | 0.19 | 0.02 | 0.3 |
| 1 | 5F1 | gH | 0.21 | 0.05 | 0.3 |
| 1 | 6B4 | gH | nd | 0.11 | 2 |

⁽¹⁾Values indicating the concentration of antibody in μg/ml required to give a 50% reduction of hCMV (VR1814) infection of primary cells. HUVEC, human umbilical vein endothelial cells, Mo-DC, monocyte-derived dendritic cells, BM-MSC, mesenchymal bone-marrow stromal cells.

### Example 2

### Identification of the Target Antigens Recognized by the Monoclonal Antibodies

To map the specificity of the hCMV neutralizing antibodies, HEK293T cells were transfected with one or more vectors encoding full length hCMV proteins UL128, UL130, UL131A, gH, gL, gB, gM, and gN. After 36 h, cells were fixed, permeabilized and stained with the human monoclonal antibodies followed by goat anti-human IgG. U.S. Pat. No. 8,603,480, which is incorporated by reference, shows the binding of representative antibodies to HEK293T cells expressing one or more hCMV proteins. Table 6 herein shows the staining pattern of all the different antibodies to hCMV gene-transfected HEK293T cells. With the exception of antibody 15D8, that stained UL128-transfected cells, all the other Group 2 antibodies did not stain single gene transfectants, suggesting that they may recognize epitopes that require co-expression of more than one gene product. Indeed, five antibodies (4N10, 10F7, 10P3, 4I22 and 8L13) stained cells co-expressing UL130 and UL131A, six antibodies (2C12, 7B13, 7113, 8C15, 8J16 and 916) stained cells co-expressing UL128, UL130 and UL131A, and one antibody (8I21) stained cells transfected with UL128 and UL130 as well as with gH and gL. All these antibodies also stained HEK293T cells transfected with all genes forming the gH/gL/UL128-130 complex. Among the Group 1 antibodies, three (11B12, 13H11 and 3G16) stained cells expressing the hCMV protein gH, six (7H3, 10C6, 5F1, 6B4, 4H9 and 2B11) stained cells expressing the hCMV protein gB and one (6L3) stained cells coexpressing the hCMV proteins gM and gN.

TABLE 6

| | Monoclonal antibody | | | | | | |
|---|---|---|---|---|---|---|---|
| | Group 2 | | | | Group 1 | | |
| | | | 2C12 | | | 7H3 | |
| | | 4N10 | 7B13 | | | 10C6 | |
| | | 10F7 | 7I13 | | | 5F1 | |
| | | 10P3 | 8C15 | | 11B12 | 6B4 | |
| HEK293T cells | | 4I22 | 8J16 | | 13H11 | 4H9 | |
| transfected with: | 15D8 | 8L13 | 9I6 | 8I21 | 3G16 | 2B11 | 6L3 |
| UL128 | + | − | − | − | − | − | nd⁽¹⁾ |
| UL130 | − | − | − | − | − | − | nd |
| UL131A | − | − | − | − | − | − | nd |
| UL128 + UL130 | + | − | − | − | − | − | nd |
| UL128 + UL131A | + | − | − | − | − | − | nd |
| UL130 + UL131A | − | + | − | − | − | − | nd |
| UL128 + UL130 + | + | + | + | − | − | − | − |
| UL131A | | | | | | | |
| gH | − | − | − | − | + | − | − |
| gH + gL | − | − | − | − | + | − | − |
| gH + UL128 + | + | + | + | − | + | nd | nd |
| UL130 + UL131A | | | | | | | |
| gL + UL128 + | + | + | + | − | − | nd | nd |
| UL130 + UL131A | | | | | | | |
| gH + gL + UL128 | + | − | − | − | + | nd | nd |
| gH + gL + UL130 | − | − | − | − | + | nd | nd |
| gH + gL + UL131A | − | − | − | − | + | nd | nd |
| gH + gL + UL128 + | + | − | − | + | + | nd | nd |
| UL130 | | | | | | | |
| gH + gL + UL128 + | + | + | + | + | + | − | − |
| UL130 + UL131A | | | | | | | |
| gB | − | − | − | nd | − | + | − |
| gM | nd | − | − | nd | nd | nd | − |
| gN | nd | − | − | nd | nd | nd | − |
| gM + gN | − | − | − | − | nd | nd | + |

⁽¹⁾nd, not done.

To further explore the identity of the antigen sites to which the antibodies bind, cross-competition experiments were performed. Here, HEK293T cells were transfected with vectors encoding full length hCMV proteins gH, gL, UL128, UL130 and UL131A. The cells were then incubated with a 20-fold excess of a competitor hCMV neutralizing antibody before addition of a biotinylated antibody. This procedure was repeated several times with different competitor antibodies and biotinylated antibodies. In these experiments four antibodies described in U.S. patent application Ser. No. 11/969,104 (11F11, 2F4 and 5A2) and U.S. patent application Ser. No. 12/174,568 (6G4) were included. The data is shown in Table 7A, B.

TABLE 7A

| Competitor | | Inhibition of binding (%) | | | | | | |
|---|---|---|---|---|---|---|---|---|
| (20-fold | | 15D8- | 4N10- | 10F7- | 4I22- | 1F11- | 2F4- | 5A2- |
| excess) | Specificity⁽¹⁾ | biotin | biotin | biotin | biotin | biotin | biotin | biotin |
| 15D8 | UL128 | 100 | 0 | 0 | 0 | 0 | 0 | 0 |
| 4N10 | UL130/UL131A | 0 | 100 | 0 | 0 | 0 | 0 | 100 |
| 10F7 | UL130/UL131A | 0 | 0 | 100 | 100 | 100 | 100 | 0 |
| 10P3 | UL130/UL131A | 0 | nd | nd | 0 | 0 | 0 | Nd |
| 4I22 | UL130/UL131A | nd | 0 | 100 | 100 | 100 | 100 | 0 |
| 8L13 | UL130/UL131A | nd | nd | 100 | nd | 100 | Nd | nd |
| 1F11 | UL130/UL131A | 0 | 0 | 100 | 100 | 100 | 100 | 0 |
| 2F4 | UL130/UL131A | nd | 0 | 100 | 100 | 100 | 100 | 0 |
| 5A2 | UL130/UL131A | nd | 100 | 0 | 0 | 0 | 50⁽²⁾ | 100 |
| 2C12 | UL128/UL130/UL131A | 0 | 0 | 0 | 0 | 0 | 0 | 0 |
| 7B13 | UL128/UL130/UL131A | nd | nd | nd | nd | nd | nd | nd |
| 7I13 | UL128/UL130/UL131A | nd | nd | nd | nd | 0 | nd | nd |
| 8C15 | UL128/UL130/UL131A | nd | nd | nd | 0 | nd | nd | nd |
| 8J16 | UL128/UL130/UL131A | nd | nd | nd | 0 | 0 | 0 | nd |
| 9I6 | UL128/UL130/UL131A | nd | nd | Nd | 0 | 0 | 0 | nd |
| 6G4 | UL128/UL130/UL131A | 0 | 0 | 0 | 0 | 0 | 0 | 0 |
| 8I21 | gH/gL/UL128/UL130 | 0 | 90 | nd | 0 | 0 | 0 | 95 |

⁽¹⁾Specificity as defined is Table 6.
⁽²⁾Competition below 100% may be due to partial overlap of epitopes or to steric hindrance or to lower affinity.

TABLE 7B

| | | Inhibition of binding (%) | | | | | |
|---|---|---|---|---|---|---|---|
| Competitor | | 2C12- | 8C15- | 8J16- | 9I6- | 6G4- | 8I21- |
| (20-fold excess) | Specificity⁽¹⁾ | biotin | biotin | biotin | biotin | biotin | biotin |
| 15D8 | UL128 | 0 | nd | nd | nd | 0 | 0 |
| 4N10 | UL130/UL131A | 0 | nd | nd | nd | 0 | 90⁽²⁾ |
| 10F7 | UL130/UL131A | 0 | nd | nd | nd | 0 | 0 |
| 10P3 | UL130/UL131A | 0 | nd | nd | nd | 0 | 0 |
| 4I22 | UL130/UL131A | 0 | nd | 0 | nd | nd | 0 |
| 8L13 | UL130/UL131A | nd | nd | nd | nd | nd | nd |
| 1F11 | UL130/UL131A | 0 | nd | nd | nd | 0 | 0 |
| 2F4 | UL130/UL131A | 0 | nd | nd | 0 | 0 | 0 |
| 5A2 | UL130/UL131A | 0 | nd | nd | 0 | 0 | 92 |
| 2C12 | UL128/UL130/UL131A | 100 | 100 | 100 | 100 | 100 | 0 |
| 7B13 | UL128/UL130/UL131A | 100 | 100 | 100 | 100 | 100 | 0 |
| 7I13 | UL128/UL130/UL131A | 0 | 0 | 0 | 0 | 0 | 0 |
| 8C15 | UL128/UL130/UL131A | 100 | 100 | 100 | 100 | 100 | 0 |
| 8J16 | UL128/UL130/UL131A | 100 | 100 | 100 | 70 | 100 | 0 |
| 9I6 | UL128/UL130/UL131A | 100 | 100 | 100 | 100 | 100 | 0 |
| 6G4 | UL128/UL130/UL131A | 100 | 100 | 100 | 100 | 100 | 0 |
| 8I21 | gH/gL/UL128/UL130 | 0 | nd | nd | nd | 0 | 100 |
| 3G16 | gH | 0 | nd | nd | nd | 0 | 0 |

⁽¹⁾Specificity as defined is Table 6. lower
⁽²⁾Competition below 100% may be due to partial overlap of epitopes or to steric hindrance or to affinity.

Based on the data in Table 7A, B, at least seven distinct antigenic sites can be distinguished on the hCMV complex formed by gH, gL, UL128 and UL130 (Table 8). Site 1 is present in UL128 and is defined by antibody 15D8. Sites 2 to 4 are formed by the combination of UL130 and UL131A and are defined by the antibodies 10F7 4122, 8L13, 1F11 and 2F4 (site 2), by 4N10 and 5A2 (site 3), and by 10P3 (site 4), respectively. Sites 5 and 6 are formed by the combination of UL128, UL130 and UL131A and are defined by antibodies 2C12, 7B13, 8C15, 8J16, 9I6 and 6G4 (site 5) and by 7I13 (site 6), respectively. Finally, site 7 is formed by the combination of gH, gL, UL128 and UL130 and is defined by the antibody 8I21. Antibodies defining site 7 and site 3 partially competed with each other, suggesting that these sites may be close in the structure of the gH/gL/UL128-131A complex.

It is anticipated that neutralizing antibodies targeted to different epitopes on the same target can be used in combination to achieve robust neutralization of virus infection, as exemplified by 10F7 and 4N10 or by 8J16 and 7I13. Moreover, it is anticipated that neutralizing antibodies targeted to different target molecules or combinations of target molecules may be used together to achieve robust virus neutralization. As one example, Table 8 suggests that 15D8 and 10F7, 15D8 and 2C12, or 8J16 and 8I21 could be combined to bring about additive or synergenic hCMV neutralization effects.

TABLE 8

| | | Antibodies defining the |
|---|---|---|
| Target antigen | Antigenic site | antigenic site |
| UL128 | 1 | 15D8 |
| UL130/UL131A | 2 | 10F7, 4I22, 8L13, 1F11, 2F4 |
| UL130/UL131A | 3 | 4N10, 5A2 |
| UL130/UL131A | 4 | 10P3 |
| UL128/UL130/UL131A | 5 | 2C12, 7B13, 8C15, 8J16, 916, |
| | | 6G4 |
| UL128/UL130/UL131A | 6 | 7I13 |
| gH/gL/UL128/UL130 | 7 | 8I21 |

In a manner similar to what is described in Table 7, HEK293T cells were transfected with a vector encoding full length gH to examine the cross-competition binding of the anti-gH antibodies. As can be seen in FIG. 2A of U.S. Pat. No. 8,603,480, which is incorporated by reference, and Table 9, at least two different binding sites were identified in the hCMV gH protein. The antibody 3G16 defines one site and the antibodies 11B12 and 13H11 define a second site. Finally, HEK293T cells were transfected with a vector encoding full length gB to examine the cross-competition binding of the anti-gB antibodies. As can be seen in FIG. 2B of U.S. Pat. No. 8,603,480 and Table 10, at least three different antigenic sites were identified in the hCMV gB protein. The antibody 6B4 defines one site, 7H3 defines a second site and the set of 10C6, 5F1, 4H9 and 2B11 define a third site. Antibody 6B4 (recognizing gB site 1) reacted by ELISA with the gB 69-78 peptide (EC₅₀ of 0.044 μg/ml). It is anticipated that antibodies that target different sites even on the same target molecule can be used in combination to achieve robust virus neutralization. It is anticipated that antibodies that target different sites even on the same target molecule can be used in combination to achieve robust virus neutralization.

TABLE 9

| | | Inhibition of binding (%) of: | | | |
|---|---|---|---|---|---|
| Competitor | | 3G16- | 11B12- | 13H11- | Antigenic |
| 20-fold excess | Specificity⁽¹⁾ | biotin | biotin | biotin | site in gH |
| 3G16 | gH | 100 | 0 | 0 | 1 |
| 11B12 | gH | 0 | 100 | 100 | 2 |
| 13H11 | gH | 0 | 100 | 100 | 2 |

⁽¹⁾As defined in Table 6.

TABLE 10

| | | Inhibition of binding (%) of: | | | | | | |
|---|---|---|---|---|---|---|---|---|
| Competitor | | 7H3- | 10C6- | 5F1- | 6B4- | 4H9- | 2B11- | Antigenic site |
| (20-fold excess) | Specificity⁽¹⁾ | biotin | biotin | biotin | biotin | biotin | biotin | in gB |
| 6B4 | gB | 0 | 0 | 0 | 100 | 0 | 0 | 1 |
| 7H3 | gB | 100 | 0 | 0 | 0 | 0 | 0 | 2 |
| 10C6 | gB | 0 | 100 | 100 | 0 | 100 | 100 | 3 |
| 5F1 | gB | 0 | 100 | 100 | 0 | 100 | 100 | 3 |
| 4H9 | gB | 0 | 100 | 100 | 0 | 100 | 100 | 3 |
| 2B11 | gB | 0 | 100 | 100 | 0 | 100 | 100 | 3 |

⁽¹⁾As defined in Table 6.
⁽²⁾Competition below 100% may be due to partial overlap of epitopes, to steric hindrance or to lower affinity.

To summarize, 15D8 binds to an epitope in UL128 that is distinct from the epitope recognized by 2C12, 7B13, 6G4 (all specific for a combination of UL128, UL130 and UL131A) and from the epitope recognized by 8I21 (specific for a combination of gH, gL, UL128 and UL130). In addition binding of 15D8 to its epitope is not inhibited by 4N10, 10F7, 10P3 and 1F11 (all specific for a combination of UL130 and UL131A).

4N10 binds to an epitope which requires expression of UL130 and UL131A and that is the same or largely overlapping to the epitopes recognized by 5A2 (specific for a combination of UL130 and UL13 1A) and 8I21 (specific for a combination of gH, gL, UL128 and UL130) but distinct from the epitopes recognized by 10F7, 4122, 1F11, 2F4 (all specific for a combination of UL130 and UL131A), 2C12 and 6G4 (both specific for a combination of UL128, UL130 and UL131A). In addition binding of 4N10 to its epitope is not inhibited by 15D8 (specific for UL128).

10F7 binds to an epitope which requires expression of UL130 and UL131A that is the same or largely overlapping to the epitope(s) recognized by 4122, 8L13, 1F11 and 2F4 but distinct from epitope(s) recognized by 4N10 and 5A2 (both specific for a combination of UL130 and UL13 1A) as well as distinct from epitopes recognized by 2C12 and 6G4 (both specific for a combination of UL128, UL130 and UL131A). In addition binding of 10F7 to its epitope is not inhibited by 15D8 (specific for UL128) or by 13H11 (specific for gH).

4I22 binds to an epitope which requires expression of UL130 and UL131A and that is the same or partially overlapping to epitope(s) recognized by 2F4, 1F11 and 10F7 but distinct from epitope(s) recognized by 4N10, 10P3 and 5A2 (all specific for a combination of UL130 and UL131A) as well as distinct from the epitopes recognized by 2C12, 8C15, 8J16, 9I6, 6G4 (all specific for a combination of UL128, UL130 and UL13 1A) and 8I21 (specific for a combination of gH, gL, UL128 and UL130. In addition binding of 4I22 to its epitope is not inhibited by the antibodies 15D8 (specific for UL128) or by 13H11 (specific for gH).

2C12 binds to an epitope which requires expression of hCMV UL128, UL130 and UL131A gene products and that is the same or largely overlapping to epitope(s) recognized by 7B13, 8C15, 8J16, 9I6 and 6G4 but distinct from the epitope recognized by 7I13 (all specific for a combination of UL128, UL130 and UL131A) and distinct from epitope(s) recognized by 15D8 (specific for UL128), 4N10, 10F7, 10P3, 4122, 8L13, 1F11, 2F4, 5A2 (all specific fora combination of UL130 and UL131A) and 8I21 (specific for a combination of gH, gL, UL128 and UL130). In addition binding of 2C12 to its epitope is not inhibited by 3G16 (specific for gH).

8C15 binds to an epitope which requires expression of hCMV UL128, UL130 and UL131A gene products and that is the same or largely overlapping to epitope(s) recognized by 2C12, 7B13, 8J16, 916 and 6G4 but distinct from the epitope recognized by 7I13 (all specific for a combination of UL128, UL130 and UL13 1A).

8J16 binds to an epitope which requires expression of hCMV UL128, UL130 and UL131A gene products and that is the same or largely overlapping to epitope(s) recognized by 2C12, 7B13, 8C15, 9I6 and 6G4, but distinct from the epitope recognized by 7I13 (all specific for a combination of UL128, UL130 and UL13 1A) and from the epitope recognized by 4I22 (specific for a combination of UL130 and UL131A).

9I6 binds to an epitope which requires expression of hCMV UL128, UL13 0 and UL131A gene products and that is the same or largely overlapping to epitope(s) recognized by 2C12, 7B13, 8C15, 8J16 and 6G4 but distinct from the epitope recognized by 7I13 (all specific for a combination of UL128, UL130 and UL131A) and from the epitope(s) recognized by 2F4 and 5A2 (specific for a combination of UL130 and UL13 1A).

8I21 binds to an epitope which requires expression of hCMV gH, gL, UL128 and UL130 gene products and that may be partially overlapping to epitope(s) recognized by 4N10 and 5A2 (both specific for a combination of UL130 and UL131A) but distinct from epitopes recognized by 15D8 (specific UL128), 10F7, 10P3, 4I22, 1F11, 2F4 (all specific for a combination of UL130 and UL131A), 2C12, 7B13, 7I13, 8C15, 8J16, 9I6 and 6G4 (all specific fora combination of UL128, UL130 and UL131A). In addition binding of 8I21 to its epitope is not inhibited by 3G16 (specific for gH).

3G16 binds to an epitope in gH that is distinct from the epitope(s) recognized by 11B12 and 13H11 (both specific for gH).

11B12 binds to an epitope in gH that is the same or largely overlapping to the epitope recognized by 13H11 and distinct from the epitopes recognized by 3G16 (both specific for gH).

13H11 binds to an epitope in gH that is the same or largely overlapping to the epitope recognized by 11B12 and distinct from the epitopes recognized by 3G16 (both specific for gH).

6B4 recognizes an epitope in gB that is distinct from the epitope(s) recognized by 7H3, 4H9, 5F1, 10C6 and 2B11 (all specific for gB).

7H3 binds to an epitope in gB that is distinct from the epitope(s) recognized by 6B4, 4H9, 5F1, 10C6 and 2B11 (all specific for gB).

10C6 binds to an epitope in gB that is the same or partially overlapping to the epitope(s) recognized by 5F1, 4H9 and 2B11, but distinct from the epitope(s) recognized by 7H3 and 6B4 (all specific for gB).

5F1 binds to an epitope in gB that is the same or largely overlapping to the epitope(s) recognized by 1006, 4H9 and 2B11 but distinct from the epitope(s) recognized by 6B4 and 7H3 (all specific for gH).

4H9 binds to an epitope in gB that is the same or largely overlapping to the epitope(s) recognized by 5F1, 10C6 and 2B11, but distinct from the epitope(s) recognized by 6B4 and 7H3 (all specific for gH).

2B11 binds to an epitope in gB that is the same or largely overlapping to the epitope(s) recognized by 5F1, 10C6 and 4H9 but distinct from the epitope(s) recognized by 6B4 and 7H3 (all specific for gH).

### Example 3

### Selection of a Combination of HCV Antibodies

Various individual antibodies disclosed herein and in U.S. Pat. No. 8,603,480, which is incorporated by reference, were profiled in vitro for their antiviral effect and off-target effects, and in silico for their developability. In addition, antibody combinations were analyzed regarding their neutralization capacity and prevention of viral escape mutations. The combinations included those comprising one antibody which bound to one subset of hCMV proteins and one that bound to another subset of hCMV proteins, such as an antibody which bound gB and an antibody which bound a multi-protein complex.

The individual antibodies 7H3 and 4I22 and the combination thereof were shown to be excellent candidates in all tested aspects. They were found to be effective binders to hCMV glycoproteins with excellent neutralization potency on clinical isolates of virus across clinically relevant primary cell types (e.g., renal and placental cell types). They effectively blocked syncytia formation and with that cell-to-cell spread of virus. In combination, these antibodies prevented the development of escape mutants over a period of more than a year. Unlike some of the other antibodies, antibodies 7H3 and 4I22 did not contain developability constrains like glycosylation sites, deamidation sites, or unlinked cys residues and no off-target binding to protein chips or various non-infected tissues was observed. All features together made the combination of 7H3 and 4I22 excellent compared to other antibodies. These features are detailed herein and below.

### Example 4

### Various Qualities and Efficacy of the Combination of 7H3 and 4I22

7H3 and 4I22 are antibodies that bind to and inhibit the function of viral glycoproteins essential for hCMV infectivity. 7H3 inhibits gB function while 4I22 inhibits the function of the 5-member complex. The combination of 7H3 and 4I22 (7H3/4122) neutralizes hCMV infection of all cell types tested by both blocking the initial infection of cells and the subsequent cell to cell spread of virus. In addition, the combination shows a marked decrease in viral resistance that is seen with single antibody therapy.

Modeling predicts that the affinity of antibody-glycoprotein interactions could be a factor in decreasing viral replication. An enzyme-linked immunosorbent assay (ELISA) using a soluble gB ectodomain expressed in a mammalian cell line was used to assess the affinity of 7H3. To assess the affinity of 4I22, a soluble 5-member complex was generated. Biacore technology, which is based on measuring differences in surface plasmon resonance, was used to measure the binding kinetics of this antibody. Both 7H3 and 4I22 bound to their respective targets with high affinity. The equilibrium dissociation constants (KD) for 7H3 and 4I22 were 289.9 pM and 310 pM, respectively.

With non-overlapping resistance mechanisms (7H3 can neutralize 4I22-resistant virus and 4I22 can neutralize 7H3-resistant virus), the rate for developing resistance to both 7H3 and 4I22 when dosed together was hypothesized to be the product of the two rates for each antibody alone. The minimal viral glycoproteins required for 7H3 and 4I22 binding were identified by transfection of HEK293T cells with genes that encode each putative glycoprotein. 7H3 binding required expression of gB whereas 4I22 binding required the expression of both UL130 and UL131A, which are essential components of the 5-member complex (Macagno et al 2010). Epitope binding experiments were used to define the epitopes recognized by 7H3 and 4I22. For these studies, cells transfected with the genes for either gB or the 5-member complex were incubated with 20-fold excess of unlabeled competitor antibodies (each with well-defined epitopes) and then biotin-labeled 7H3 or 4I22. These studies demonstrated that 7H3 recognizes a conformational epitope located in the N-terminus ectodomain of gB while 4I22 recognizes a conformational epitope formed by the UL130 and UL131A components of the 5-member complex. To define the antibody-antigen contact residues, selection of hCMV resistant to neutralization with 7H3 or 4I22 was performed, and mutations correlating with reductions in susceptibility to antibody inhibition were identified. In the presence of 7H3, passage of hCMV in fibroblasts resulted in virus with >41-fold increases in EC50 to 7H3 and a double mutation in the N-terminus ectodomain of gB (E361K and D362N) whereas passage in epithelial cells resulted in virus with a 22-fold decrease in susceptibility and a single gB amino acid deletion (E381 deletion). In the presence of 4I22, passage of hCMV in epithelial cells resulted in virus with >50,000-fold decrease in susceptibility to 4I22 with one of two mutations detected singly in UL130 (Q191K or W179R). Decreased susceptibility to either 7H3 or 4I22 typically developed after 76 to 158 days of passage.

In contrast, no escape virus has thus far been generated in the presence of both antibodies after 439 days of continuous culture. Thus, the combination provides the unexpected result of very low viral resistance even after long term administration of the therapy.

### Example 5

### Neutralization of CMV in Multiple Cell Types

The ability of 7H3 and 4I22 to neutralize the infection of different clinically relevant cell types by the different clinical strains of hCMV was tested, as previously described (Manley et al 2011). After mixing virus with antibody at concentrations ranging from 0.001 to 10,000 μg/mL for 1 hour, the virus and antibody mixture was incubated with permissive cells for 3 hours. The cells were then washed, incubated for 24 hours, fixed and stained for immediate early (IE) gene products as a marker for viral entry and initiation of productive replication. Subsequently, the EC90 of 7H3 and 4I22 as well as hCMV hyperimmune globulin was calculated from the percent of IE positive nuclei among the total number cells stained with 4′, 6-diamidino-2-phenylindole (DAPI) using high content imaging. Table 11 shows data testing the ability of 7H3 and 4I22 to neutralize the infection of 10 different cell types by the clinical strain VR1814. 7H3 and 4I22 neutralized hCMV infections of primary epithelial and endothelial cells. 7H3 was approximately 10-fold more potent than hCMV hyperimmune globulin while 4I22 was 100-to 1000-fold more potent. 7H3 also neutralized hCMV infection of primary fibroblasts. In this cell type, 7H3 was 100-to 1000-fold more potent than hCMV hyperimmune globulin. As expected, 4I22 did not neutralize hCMV infection of primary fibroblasts because the 5-member complex is not required for viral entry into fibroblasts.

TABLE 11
Ability of 7H3 and 4I22 to neutralize hCMV
infection of different clinical cell types
EC90 (μg/mL) of different antibodies to neutralize
infection of different cell types

| | | | hCMV hyper |
|---|---|---|---|
| Cell type | 7H3 | 4I22 | immune globulin |
| Adult retinal pigment | 2.37 | 0.005 | 13.81 |
| epithelial | | | |
| Renal medullary epithelial | 8.68 | 0.149 | 75.19 |
| Renal cortical epithelial | 2.33 | 0.012 | 31.84 |
| Renal proximal tubule | 4.9 | 0.022 | 57.69 |
| epithelial | | | |
| Placental epithelial | 6.64 | 0.043 | 70.76 |
| Uterine microvascular | 2.96 | 0.019 | 34.69 |
| endothelial | | | |
| Human umbelical vein | 3.77 | 0.008 | 25.09 |
| endothelial | | | |
| Human coronary artery | 3.01 | 0.009 | 18.92 |
| endothelial | | | |
| Placental fibroblasts | 10.35 | >100* | 3432.99 |
| Neonatal normal human | 2.81 | >100* | 1264.35 |
| dermal fibroblasts | | | |

*For experiments using fibroblasts, concentrations of 4I22 did not exceed 100 μg/mL.

### Example 6

### Neutralization of CMV Strains

There are numerous different clinical isolates of CMV available. Tables 12 and 13 show data testing the ability of 7H3 and 4I22 to neutralize the infection of the specified cell types by 21 different clinical isolates of hCMV.

TABLE 12
Ability of 7H3 and 4I22 to neutralize infection (μg/mL) of adult
retinal pigment epithelial cells by different clinical hCMV strains

| | | | | hCMV |
|---|---|---|---|---|
| | Year of | | | hyperimuune |
| hCMV isolate and Origin | isolation | 7H3 | 4I22 | globulin |
| 8816 Massachusetts, US | 2006 | 0.75 | 0.011 | 12.8 |
| 8817 Massachusetts, US | 2006 | 1.34 | 0.006 | 13.1 |
| 8818 Massachusetts, US | 2006 | 2.47 | 0.025 | 66.8 |
| 8819 Massachusetts, US | 2007 | 2.11 | 0.015 | 23.1 |
| 8821 Massachusetts, US | 2007 | 3.79 | 0.021 | 13.0 |
| 8822 Massachusetts, US | 2007 | 1.30 | 0.029 | 13.7 |
| 8824 Massachusetts, US | 2007 | 0.98 | 0.033 | 34.2 |
| MP-LTN-901* Alabama, US | 2012 | 0.61 | 0.023 | 14.3 |
| MP-LW-1802* Alabama, US | 2013 | 6.36 | 0.011 | 20.9 |
| MP-LTN-101 Alabama, US | 2013 | 0.18 | 0.049 | 12.0 |
| TR California, US | 1998 | 0.62 | 0.005 | 5.6 |
| TM-E5271* Germany | 2000 | 0.17 | 0.010 | 23.9 |
| TM-E28257* Germany | 2011 | 1.16 | 0.009 | 10.0 |
| TM-E20744* Germany | 2012 | 1.37 | 0.010 | 24.0 |
| TM-31354 Germany | 2005 | 1.42 | 0.028 | 14.8 |
| TM-E14953* Germany | 2002 | 1.09 | 0.012 | 11.4 |
| TM-20749 Germany | 1999 | 4.36 | 0.010 | 10.2 |
| TM-E19664* Germany | 2002 | 0.26 | 0.016 | 22.1 |
| TM-E28175 Germany | 2003 | 0.24 | 0.006 | 20.1 |
| TM-E9361* Germany | 2001 | 0.33 | 0.002 | 12.5 |
| VR1814 Italy | 2001 | 2.37 | 0.005 | 13.8 |

*Positive cells are counted manually due to the low infection rate in the control wells.

TABLE 13
Ability of 7H3 and 4I22 to neutralize infection
EC90 (μg/mL) of human umbilical vein endothelial
cells by different clinical hCMV strains

| | | | | hCMV |
|---|---|---|---|---|
| | Year of | | | hyperimmune |
| hCMV isolate and Origin | isolation | 7H3 | 4I22 | globulin |
| 8816 Massachusetts, US | 2006 | 3.75 | 0.008 | 41.2 |
| 8817 Massachusetts, US | 2006 | 2.31 | 0.024 | 45.4 |
| 8818 Massachusetts, US | 2006 | 9.9 | 0.055 | 91.2 |
| 8819 Massachusetts, US | 2007 | 5.4 | 0.015 | 46.3 |
| 8821 Massachusetts, US | 2007 | 5.72 | 0.038 | 55.8 |
| 8822 Massachusetts, US | 2007 | 6.23 | 0.014 | 55.4 |
| 8824 Massachusetts, US | 2007 | 4.77 | 0.053 | 44.1 |
| MP-LTN-901 Alabama, US | 2012 | 1.32 | 0.053 | 100.2 |
| MP-LW-1802* Alabama, US | 2013 | 0.12 | 0.001 | 1319.1 |
| MP-LTN-101 Alabama, US | 2013 | 0.02 | 0.001 | 4.0 |
| TR California, US | 1998 | 0.35 | 0.007 | 26.9 |
| TM-E5271* Germany | 2000 | 0.19 | 0.001 | 15.5 |
| TM-E28257* Germany | 2011 | 1.02 | 0.001 | 10.1 |
| TM-E20744 Germany | 2012 | 0.39 | 0.020 | 34.0 |
| TM-31354* Germany | 2005 | 2.20 | 0.012 | 13.5 |
| TM-E14953 Germany | 2002 | 0.12 | 0.012 | 363.4 |
| TM-20749* Germany | 1999 | 1.18 | 0.011 | 16.7 |
| TM-E19664 Germany | 2002 | 0.31 | 0.013 | 25.1 |
| TM-E28175 Germany | 2003 | 0.28 | 0.009 | 119.6 |
| TM-E9361* Germany | 2001 | 0.29 | 0.016 | 20.6 |
| VR1814 Italy | 2001 | 3.77 | 0.008 | 25.0 |

*Positive cells are counted manually due to the low infection rate in the control wells.

Both antibodies could neutralize the infection of adult retinal pigment epithelial cells (Table 12), human umbilical vein endothelial cells (Table 13), and neonatal normal human dermal fibroblast cells (data not shown) by geographically and temporally distinct clinical hCMV isolates. 7H3 was approximately 10-fold more potent than hCMV hyperimmune globulin while 4I22 was 100-to 1000-fold more potent. This data shows that the 7H3 and 4I22 antibodies were effective in neutralizing different CMV isolates as single agents and that the combination and dosing of these antibodies would be efficacious and while reducing viral resistance.

### Example 7

### Combination of Antibodies and Synergy

Using multiple different concentrations of each antibody, the effects of 7H3 and 4I22 in combination were assessed using the Loewe Additivity, Highest Single Agent, and Bliss Independence models of synergy. Three dimensional surface plots of synergy volumes demonstrated that 7H3 and 4I22 in combination were additive to slightly synergistic in neutralization of hCMV infection of epithelial and endothelial cell lines. Notably, no antagonism was observed with the antibodies in combination. To test the ability of the monoclonal antibodies to suppress viral replication over an extended time period, adult retinal pigment epithelial 19 cells were inoculated with hCMV and cultured in the presence of 7H3 or 4I22 at approximately 1-and 10-times the EC90 concentrations (5 μg/mL and 50 μg/mL for 7H3 and 0.01 μg/mL and 0.1 μg/mL for 4I22) for up to 28 days. Monitoring viral replication by immunostaining and visualizing cytopathic effects indicated that 7H3 (at 1-or 10-times the EC₉₀) or 4I22 (at 10-times the EC₉₀) effectively blocked hCMV replication for at least 28 days and that the combination of the two antibodies was more effective than the individual antibodies at the same total selection pressure, e.g., fold EC50 concentration. Cell-to-cell neutralization hCMV infected cells can fuse with uninfected neighboring cells, forming syncytia and allowing virus to spread to the uninfected cell. Cell-cell fusion has been suggested as the primary mechanism by which hCMV is transferred between monocytes and endothelial cells, facilitating systemic dissemination in humans (Waldman et al 1995, Hahn et al 2004, Bentz et al 2006). Like virus-cell fusion, cell-cell fusion is also mediated by viral glycoproteins but not necessarily by the same domains of those glycoproteins in common to both processes. A quantifiable cell-cell fusion assay was used to test if 7H3 and 4I22 could inhibit syncytia formation. Adenoviruses were constructed that expressed the hCMV glycoproteins with a known role in virus-cell fusion. Viral interference assays and flow cytometry with conformation specific antibodies against the glycoproteins were used to demonstrate that the glycoproteins were expressed on the surface of adenoviral transduced epithelial cells. Cells expressing gB and the 5-member complex readily fused. For both 7H3 and 4I22, dose-dependent inhibition of cell-cell fusion of cells expressing gB and the 5-member complex was noted. Both antibodies were significantly more potent at inhibiting fusion than hCMV hyperimmune globulin. The 7H3 antibody had an EC50 of 4.77 μg/ml, and 4I22 had an EC50 of 0.076 μg/ml while the control hCMV hyperimmune globulin had an EC50 of 311.34

### Example 8

### Viral Resistance

hCMV isolates with reduced susceptibility to 7H3 or 4I22 were selected in vitro after serial passage of virus in the presence of either antibody alone.

For 7H3, emergence of virus with reduced susceptibility correlated with the detection of mutations mapping to gB and were dependent on the cell-type used during serial passage. A gB E381 deletion was selected after passage in epithelial cells while E361K and D362N mutations were selected after passage in fibroblasts. Two gB sequences contained a D362E substitution, and the susceptibility to the 7H3 antibody was comparable to the other strains including VR1814.

For 4I22, reduced viral susceptibility after passage in epithelial cells correlated with detection of Q191K mutations in UL130. No mutations were identified in gH, gL, UL128, or UL131A. Passage of hCMV in the presence of 1F11, an antibody that competes for binding with 4I22, resulted in the selection of one of two different resistance mutations within UL130, D185N or Q191R. 4I22 cannot neutralize virus with either of these mutations indicating cross-resistance with these variants. None of the identified mutations in UL130 were detected in any of 21 successfully sequenced hCMV clinical isolates.

No cross resistance was observed between 7H3 and 4I22. Viruses displaying reduced susceptibility following selection with one antibody remained susceptible to the other antibody at concentrations similar to those required to inhibit wild-type virus. Also, 7H3 and 4I22 are not cross-resistant with the nucleoside inhibitor ganciclovir, as viruses with reduced susceptibility to 7H3 or 4I22 remain susceptible to ganciclovir in vitro. MSL-109 is an IgG1 monoclonal antibody that recognizes an epitope in hCMV gH. MSL-109 neutralized laboratory and clinical hCMV strains in vitro. During clinical trials (Boeckh et al 2001), hCMV isolated from MSL-109-treated stem cell transplant recipients suggested that the virus had developed resistance to the antibody. Studies demonstrated that the viral resistance was the result of a non-genetic escape mechanism in which MSL-109 is taken up by hCMV infected cells and incorporated into the envelope of virions in a dose-dependent manner. The virus subsequently used the Fc domain of the incorporated MSL-109 to infect other cells (Manley et al 2011). hCMV exposed to 7H3 or 4I22 did not develop resistance via this mechanism as virus was not able to escape antibody inhibition in a single passage in a dose-dependent manner and did not require involvement of the Fc region to mediate reduced susceptibility. These data indicate that resistance to 7H3 and 4I22 occurs via mechanisms distinct from that observed for MSL-109. 7H3 does not utilize the non-genetic escape mechanism that was observed for MSL-109. These data show that CMV mutants arising from the administration of a single antibody would be neutralized by the other antibody in the 7H3/4122 combination.

Viruses with reduced susceptibility to 7H3 and 4I22 in combination could not be isolated. Compared to the individual antibodies, passaging in ARPE-19 epithelial cells in the presence of 7H3 and 4I22 in combination inhibited viral infection to a greater extent. This was indicated by a significant delay in the appearance of CPE and much lower viral titers at each round of propagation (1×10² to 1×10³ infectious units [IU]/mL) than typical for wild type VR1814 (1×10⁶ to 1×10⁷ IU/mL). After 439 days in culture, titers were too low for the virus to be analyzed in the neutralization assay. However, it was possible to able to PCR amplify gB, gH, gL and UL128-131A; no mutations in these genes were detected.

The roles of the resistance-associated mutations by engineering each variant (UL130 Q191K, gB E361K, gB D362N, gB E361K+D362N, and gB E381 deletion) into HCMV strain AD169 using BAC mutagenesis were investigated. The wild-type and mutant BAC-derived viruses reached comparable titers in culture (data not shown). The ability of 7H3 and 4I22 to neutralize wild-type and mutant BAC-derived virus was then compared. When introduced individually into the HCMV genome, two of the gB mutations observed on selection with 7H3 were found to result in decreased susceptibility to the antibody (E361K and E381 deletion). In contrast, the single D362N mutation conferred no significant resistance. Both mutations together, however, reduced susceptibility more than the E361K variant alone. The UL130 mutation identified on passage in the presence of 4I22 (Q191K) also resulted in a decrease in susceptibility to the antibody.

HCMV passaged in the presence of 7H3 or 4I22 remains susceptible to the non-selecting monoclonal antibody. Pooled HCMV virus resistant to 7H3 after passage in the presence of antibody on fibroblasts (NHDF cells), was tested for neutralization by 7H3 on NHDF cells and 4I22 on epithelial cells (ARPE-19 cells). As expected, the virus had reduced susceptibility to 7H3; however, it remained susceptible to 4I22. Similarly, pooled virus resistant to 7H3 after passage in epithelial cells was not as readily neutralized by 7H3 on NHDF cells and remained sensitive to 4I22 on ARPE-19 cells. Virus resistant to 4I22 after passage in epithelial cells showed decreased susceptibility to 4I22 on ARPE-19 cells but remained susceptible to 7H3 on ARPE-19 cells. These results indicate an absence of cross resistance between 7H3 and 4I22 monoclonal antibodies, consistent with the antibodies targeting distinct glycoproteins and consistent with the reduction in viral resistance seen with the combination of the two antibodies.

### Example 9

### Safety Profile of the Antibody Combination in Rats

The potential for 7H3 and 4I22 to exhibit off-target binding was initially assessed using two protein-binding microarray assays. The Protagen® protein chip assay contains 384 intracellular and secreted human proteins expressed in bacterial cells (Protagen, Dortmund, Germany). An in-house assay contains 50 human proteins expressed in insect cells. No significant binding to any antigen was observed for either 7H3 or 4I22.

A single 4 week study with weekly IV dosing of 7H3 and 4I22 was conducted in the rat to support first-in-human administration. In this study, specific assessments of safety pharmacological end-points (such as cardiovascular, central nervous system, and respiratory) were not included due to the lack of target in rats, lack of non-specific binding to rat tissues, and lack of pharmacological activity and relevance in the rat. No clinical signs or changes in hematology or clinical chemistry were noted in the study indicating any effects of the antibodies on cardiovascular, central nervous system, or respiratory function.

Concentrations of the monoclonal antibodies in rat serum were determined using a sandwich Meso Scale Discovery® (MSD, Rockville, Md.)-based method in the GLP toxicology study for 7H3 and 4I22 or a high-performance liquid chromatography with tandem mass spectrometry (HPLC-MS/MS)-based method in the earlier dose-range finding study for 4I22. The MSD®-based assays used anti-idiotypic mouse monoclonal antibodies against either 7H3 or 4I22 to allow specific determination of the two antibodies from the same serum sample. The lower limits of quantification (LLOQ) for the assays are 10 ng/mL for 7H3 and 100 ng/mL for 4I22.

An HPLC-MS/MS assay based on the unique amino acid sequence at the complementarity determining region was developed to quantify 4I22 in serum samples. The presence of anti-drug antibodies against 7H3 or 4I22 was evaluated in rat serum using MSD®-based bridging assays in which the specific antibody (7H3 or 4I22) was used both as the capture and the detection reagent. A mouse anti-human IgG monoclonal antibody was used as a non-drug-specific positive control antibody. The sensitivity of the assay was 15 ng/mL for the positive control antibody in rat serum, with drug tolerances of 240 μg/mL for 7H3 and 44.3 μg/mL for 4I22. Both assays were validated in compliance with regulatory guidelines.

### Example 10

### Pharmacokinetics in Rats

The pharmacokinetic (PK) profile of 7H3/4122 (7H3 and 4122) was evaluated in a 4-week GLP toxicology study in rats following 5 weekly intravenous doses. Serial PK blood samples were collected from 4 to 12 animals at each time point. Samples were obtained around the first and fifth doses. The PK profile of 4I22 was also assessed in a 2-week non-GLP dose-range finding study in rats following 3 weekly intravenous doses. Serial PK blood samples were collected from 2 animals at each time point. In the 4-week toxicology study, both 7H3 and 4I22 exhibited typical IgG1 PK profiles, with observed Cmax values at the first sampling time point (15 minutes post-dose) followed by rapid distribution phases within the first 24 hours and slower elimination phases (Table 14). The terminal half-life (T_{1/2}) values were 9.52 and 11.5 days for 7H3 and 4I22, respectively. Exposure to both antibodies increased in an approximately dose proportional manner, indicating linear kinetics. As expected based on the elimination T_{1/2} values of 9.52 and 11.5 days, accumulation was observed for both antibodies. No pronounced gender-based PK differences were observed.

TABLE 14
Mean non-compartmental PK parameters of 7H3 and
4I22 following intravenous administration in rats

| | Dose | Tmax | Cmax | Cmin | AUC_{0-7 d} | T_{1/2} |
|---|---|---|---|---|---|---|
| Antibody | (mg/kg) | (hr) | (μg/mL) | (μg/mL) | (μg-days/mL) | (days) |
| 7H3 | 50 | 0.25 | D 1: 1620 | D 8: 285 | D 1: 3140 | ND |
| | | | D 22: 1990 | D 22: 514 | D 22: 6190 | |
| | | | | D 29: 569 | | |
| | 150 | 0.25 | D 1: 3770 | D 8: 684 | D 1: 8730 | ND |
| | | | D 22: 4910 | D 22: 1030 | D 22: 13000 | |
| | | | | D 29: 1090 | | |
| | 500 | 0.25 | D 1: 14000 | D 8: 1540 | D 1: 24300 | Post D 29: 9.52 |
| | | | D 22:15300 | D 22: 2280 | D 22: 35300 | |
| | | | D 29: 16800 | D 29: 2320 | D 29: 33100 | |
| | | | | D 36: 2010 | | |
| 4I22 | 5 | 0.25 | D 1: 125* | D 8: 36.1 | D 1: 333 | ND |
| | | | D 22: 204 | D 22: 65.4 | D 22: 692 | |
| | | | | D 29: 69.9 | | |
| | 15 | 0.25 | D 1: 401 | D 8: 99.8 | D 1: 1020 | ND |
| | | | D 22: 581 | D 22: 162 | D 22: 1700 | |
| | | | | D 29: 164 | | |
| | 50 | 0.25 | D 1: 1710 | D 8: 254 | D 1: 3510 | Post D 29: 11.5 |
| | | | D 22: 2710 | D 22: 486 | D 22: 5760 | |
| | | | D 29: 1880 | D 29: 416 | D 29: 4610 | |
| | | | | D 36: 320 | | |

Cmin = trough serum concentration measured 7 days post each injection on the specified study day;
D = study day; ND = not determined.
*One animal with a value below the limit of quantification is excluded.

Using a different bioanalytical method (HPLC-MS/MS), similar results were obtained for 4I22 in the 2-week dose-range finding study.

### Example 11

### Serum Exposure in Rats

The observed serum exposures of 7H3 and 4I22 at the NOAEL defined in the 4-week toxicology study are compared in Table 15 with the predicted human PK exposures of both antibodies following IV administration every 4 weeks. Sufficient 7H3/4 22 exposure was achieved in the nonclinical toxicology study to support human doses for clinical studies (detailed herein and below).

TABLE 15
Serum exposure multiples of 7H3 and 4I22 in rat versus human

| | NOAEL | | | Human | Exposure | | |
|---|---|---|---|---|---|---|---|
| | dose | | | dose | multiple | | |
| | (mg/kg | | | (mg/kg | (rat/ | | |
| | IV | AUC0-7d | Rat | IV) | human) | Based | Based |
| Anti- | every | (μg/ | Cavg | Cmax | every 4 | on | On |
| body | week) | mL · d) | (μg/mL) | (μg/mL) | weeks) | Cavg | Cmax |
| 7H3 | 500 | 33100 | 4728.6 | 16800 | 1 | 649 | 581 |
| | | | | 50 | 13.0 | 11.6 | |
| 4I22 | 50 | 4610 | 658.6 | 1880 | 0.1 | 904 | 650 |
| | | | | 5 | 18.1 | 13.0 | |

No gender differences in PK values were observed; exposures are presented based on combined male and female data.
Rat exposures (AUC0-7d and Cmax) are based on observed values following the last IV administration on Day 29 in the 4-week toxicology study. Rat Cavg values were calculated based on AUC0-7d divided by 7.
Since the dosing frequencies are different in human vs. rat, steady state average concentrations rather than AUCs are used to calculate exposure multiples. For human Cavg and Cmax predictions, typical human IgG1 linear PK parameters were assumed for both 7H3 and 4I22 (CL = 0.2 L/d, Vc = 2.5 L, Vtissue = 2.6 L, Q = 0.5 L/d). AUC0-28d at steady state (84 to 112 days after dosing) following IV administration once every 4 weeks was calculated based on PK simulations. This value was then divided by 28 to generate the predicted human Cavg exposure.

### Example 12

### Immunogencity of the Antibodies in Rats

As fully human IgG1 monoclonal antibodies, 7H3 and 4I22 are expected to raise antidrug antibodies when dosed in non-human species. Although the immunogenicity in animals is not considered predictive of human immunogenicity, anti-7H3 and anti-4I22 antibodies were evaluated in both of the rat studies in order to help interpret the TK results. No treatment-related anti-drug antibodies were detected in any of the samples tested from the 2-week dose-range finding study and the 4-week toxicology study. Because some of the samples contained antibody concentrations that exceeded the drug tolerance levels of the assays (240 and 44.3 μg/mL for anti-7H3 and anti-4I22, respectively), the presence of an anti-drug antibody response cannot be excluded. However, the observed TK profiles indicated that exposures to the antibodies were maintained throughout the studies, making it unlikely that significant immunogenicity occurred.

### Example 13

### Toxicology in Rats

The nonclinical safety and toxicology program for 7H3/4122 combination has included tissue cross-reactivity studies in human, rat and monkey tissues and a 4-week repeat dose toxicology study in the rat with weekly dosing up to 500 mg/kg and 50 mg/kg of 7H3 and 4I22, respectively (Table 16). The rhesus CMV infection model most closely simulates human infections (Powers and Frith 2008), but the antibodies were unable to neutralize rhesus CMV in vitro. Thus, in accordance with ICH guidance S6 (R1), the toxicology program for 7H3/4I22 was restricted to one 4-week GLP study in rats in which in the combination of 7H3 and 4I22 were dosed weekly. No single-dose toxicology studies were conducted.

TABLE 16
Toxicology

| | Method of | | | | |
|---|---|---|---|---|---|
| | Administration | | | | |
| | (Vehicle/ | | Antibodies | | |
| | Formulation) | Duration | and dose | Gender and | |
| | Toxicology | of dosing | levels | number per | |
| Species | study | (Weeks) | (mg/kg) | group | Major findings |
| Rat | Intravenous | Weekly, 3 | 6B4/4I22 | 5 males | Mild alterations in |
| (non-GLP) | | doses over | 0/0, 60/6, | | prothrombin time (mid |
| | | 2 weeks | 200/20, and | | and high dose), |
| | | | 600/60 | | triglyceride (all doses) and |
| | | | | | magnesium (high dose) |
| | | | | | concentrations; mild to |
| | | | | | moderate increases in |
| | | | | | globulins (all doses). |
| | | | | | NOAEL defined as 600/60 |
| | | | | | mg/kg for 6B4/4I22 |
| Rat (GLP) | Intravenous | 5 weekly | 7H3/4I22 at | 10M + 10F per | Increases in serum |
| | | doses over | the following | dose group in the | phosphorus (males mid |
| | | concentrations | doses: 0/0, | main study; 6M + | and high doses). Increases |
| | | 4 weeks; 8- | 50/5, and | 6F per control | in globulins and total |
| | | Week | 500/50 | and high-dose | protein (males and females |
| | | Recovery | | group for | high doses). Decreases in |
| | | | | recovery part | triglycerides and |
| | | | | | bicarbonate levels (males |
| | | | | | and females high doses). |
| | | | | | Decreases in sodium and |
| | | | | | chloride concentrations |
| | | | | | (all treated males and |
| | | | | | females). NOAEL defined |
| | | | | | as 500/50 mg/kg for |
| | | | | | 7H3/4I22. |

ADCC = antibody-dependent cell-mediated cytotoxicity; GLP = good laboratory practice; ISH = in situ hybridization; NA = not applicable; NOAEL = no observed adverse effect level. 6B4 is an earlier candidate monoclonal antibody.

### Example 14

### Antibody Dependent Cell-Mediated Cytotoxicity (ADCC)

Expression of hCMV proteins on the surface of infected cells can trigger antibody-dependent cell-mediated cytotoxicity (ADCC) killing of these cells, limiting the extent of infection. hCMV expresses proteins on the surface of infected cells that can function as Fc-gamma receptors (Keller et al 1976, Murayama et al 1986, Antonsson and Johansson 2001), and these proteins are presumed to capture the Fc portion of circulating antibodies and limit the extent of ADCC during a natural infection. The potency of 7H3/4I22 for inducing ADCC was tested in vitro using the hCMV hyperimmune globulin (Cytotec®) as a reference because hCMV hyperimmune globulin has been safely administered to humans for the treatment and prevention of hCMV infections. In this in vitro study, limited ADCC was observed with hCMV-infected cells treated with 7H3 but not with 4I22. The extent of ADCC was similar to or lower than that noted with hCMV-infected cells treated with hCMV hyperimmune globulin. In contrast, the chimeric mouse/human antibody that recognizes the human epidermal growth factor receptor (Cetuximab®) was used as positive control and induced a high level of cytotoxicity. Targeting cells that express hCMV antigens for antibody-dependent destruction would likely be a benefit of 7H3/4I22 therapy by helping to destroy cells actively generating infectious virus. Only cells with actively replicating virus express hCMV glycoproteins on the cell surface, and hCMV replication is a lytic process that results in cell death. Thus, ADCC would only be expected to hasten ultimate cell death. The safety of hCMV hyperimmune globulin when administered to patients is consistent with the lack of significant ADCC or even with a possible benefit if ADCC limits hCMV replication and resulting symptoms.

### Example 15

### Tissue Cross-Reactivity in Rats and Humans

In initial non-GLP tissue-cross reactivity studies, no binding of 7H3 and 4I22 to a selected panel of rat tissues and 4I22 to rat and cynomologus monkey tissues (heart, lung, liver, kidney, spleen and brain) were observed (Table 17). These results are consistent with the absence of human hCMV glycoproteins in these species and the absence of cross-reactivity of these antibodies to CMV able to infect rats and cynomologus monkeys (Davison et al 2003, Murphy and Shenk 2008, Powers and Frith 2008).

7H3 and 4I22 exhibited no off-target binding in GLP human tissue cross-reactivity studies. Scattered binding was noted in few human tissues (lung, kidney, thymus, salivary gland, jejunum and stomach) but only occurred in tissues confirmed to be positive for hCMV DNA and

RNA by in situ hybridization. In non-GLP tissue cross-reactivity studies, no off target binding was observed from a selected panel of human adult and fetal tissues (heart, lung, liver, kidney, spleen, brain and placenta). Scattered positive stained cells were noted in some human adult tissues (heart, lung, brain, kidney, spleen and placenta) and human fetal tissues (liver, lung and brain) but only occurred to tissues confirmed to be positive for hCMV DNA and RNA by in situ hybridization.

Rats in a 4-week GLP toxicology study received 5 weekly intravenous doses of both antibodies, 7H3 and 4I22, or of vehicle, followed by an 8-week recovery period. Doses administered were 0/0, 50/5, 150/15 and 500/50 mg/kg of 7H3/4I22. No unscheduled deaths or toxicologically relevant test item-related effects on clinical signs, body weight development, ophthalmic changes, or food consumption as well as no macroscopic or microscopic findings that may have been attributed to 7H3/4I22 were noted in this study. There was no clinical pathology evidence of toxicity. The only changes noted were an increase in serum phosphorus concentrations in males dosed at >150/15 mg/kg/week, a decrease in serum triglycerides and bicarbonate levels in males and females dosed at >500/50 mg/kg/week and a decrease in sodium and chloride concentrations in male and/or female rats at 50/5, 150/15 and 500/50 mg/kg/week. An increase in globulin concentration associated with increased total protein levels and decreased albumin-globulin ratios was also observed but this was considered to be due to the detection of the test item (7H3 and 4I22 being detected by the assay and not an increase in rat globulin concentrations). None of these changes were considered to be adverse. In conclusion, no adverse effects were noted at all doses tested, including at the highest dose administered.

No evidence of immunogenicity to either antibody was noted. The NOAEL was defined as the highest dose administered (500 mg/kg of 7H3 and 50 mg/kg of 4I22). With non-overlapping resistance mechanisms (7H3 can neutralize 4I22-resistant virus and 4I22 can neutralize 7H3-resistant virus), the rate for developing resistance to both 7H3 and 4I22 when dosed together is predicted to be the product of the two rates for each antibody alone. During in vitro neutralization experiments, no resistant virus was detected with combination of 7H3 and 4I22 at concentrations at or above the EC₅₀ so for hCMV for 439 days of continuous culture.

Although viral loads in excess of 10⁸ copies of hCMV DNA/mL can be detected in transplant recipients, 7H3 and 4I22 will be administered with the specific aim of preventing significant hCMV replication from occurring (defined as viral loads greater than 10³ copies of hCMV DNA/mL), which further decreases the risk of resistance developing. For all hCMV strains, cell types (excluding fibroblasts), and dose combinations tested, 7H3 and 4I22 in combination demonstrated additive or slightly synergistic ability to inhibit hCMV replication in vitro, with no antagonism noted. Similar results were seen with other combinations, using one antibody that targets gB (such as 7H3) and another that targets gH, gH-gL, the 3-member complex or the 5-member complex (such as 4I22). Because synergy was not consistently seen, the anti-viral activities of 7H3 and 4I22 were assumed to be independent for all modeling and dose predictions.

TABLE 17
Tissue cross-reactivity
Tissue cross reactivity studies

| Species | Antibodies (mg/kg) | Major findings |
|---|---|---|
| Human, | 6B4 or 4I22 | Scattered positively stained |
| rat and | (but not 7H3); | cells in human adult heart, |
| monkey | | lung, brain, kidney, spleen |
| (non- | | and placenta and in fetal |
| GLP) | | lung and brain; positively |
| | | stained cells confirmed by |
| | | ISH to be hCMV-infected. |
| | | No staining observed in rat |
| | | or cynomolgus monkey |
| | | tissues. |
| Human | 7H3 or 4I22; | Scattered positively stained |
| and rat | | cells in human fetal liver |
| (non- | | and brain; positively stained |
| GLP) | | cells confirmed by ISH to |
| | | be hCMV-infected. |
| | | No staining observed in rat |
| | | tissues. |
| Human | 6B4 or 4I22 | Scattered positively stained |
| (GLP) | (but not 7H3); | cells in the lung, kidney and |
| | | cerebral cortex; positively |
| | | stained cells confirmed by |
| | | ISH to be hCMV-infected. |
| | | Off-target binding of |
| | | 6B4 to cells in the skin |
| | | and thymus. |
| Human | 7H3 or 4I22; | Scattered positively stained |
| (GLP) | | cells in the kidney, jejunum, |
| | | tonsil, parotid and placenta; |
| | | positively stained cells |
| | | confirmed by ISH to be |
| | | hCMV-infected. |

### Example 16

### Dosing for CMV Infection in Humans

As shown herein, including in the Examples, a combination of anti-gB (7H3) and anti-5-member complex (4I22) antibodies that can inhibit infection of fibroblasts as well as endothelial and hematopoietic cells should be able to block replication as well as systemic spread of hCMV.

The combination of 7H3/4I22 has several advantages. (1) Although 7H3 inhibited hCMV infection of all cell types tested, 4I22 is a high affinity and potency neutralizing antibody that targets the 5-member complex, which is required for the infection of cell types likely required for systemic spread of hCMV. (2) Antibodies directed against gB (such as 7H3) and the 5-member complex (such as 4122) are the predominant neutralizing antibodies detected after a natural infection. Targeting both gB and the 5-member complex will likely maximize viral neutralization and control of hCMV infections in vivo. (3) In vitro data suggest that the combination of 7H3 and 4I22 will significantly decrease the development of viral resistance to either antibody.

As detailed in Examples 10 to 15, rats in a 4-week GLP toxicology study received 5 weekly intravenous doses of both antibodies, 7H3 and 4I22, or of placebo. No adverse effects were noted at all doses tested, including at the highest dose administered: 500 mg/kg of 7H3 and 50 mg/kg of 4I22. No evidence of treatment-related immunogenicity to either antibody was noted. The pharmacokinetic (PK) profiles of 7H3 and 4I22 were typical of human IgGlantibodies, with dose-related increases in exposure, slow clearance, and long terminal elimination half-lives.

However, hCMV viral breakthrough in vitro is only fully inhibited when virus is serially passaged in the presence of 4I22 at concentrations that are at least 10-times the concentration of antibody inducing 90% neutralization (EC₉₀), indicating a need for a 10-fold increase in the dose predicted from the neutralization assays in order to suppress viral rebound in patients. For 7H3, in vitro suppression of viral breakthrough requires lower concentrations of 7H3 and these are similar to the concentrations needed in the neutralization assay. Thus, the model prediction along with the in vitro viral breakthrough data indicate that in order to durably suppress viral replication, minimal trough serum concentrations of at least about 7.4 μg/mL (for 7H3) and at least about 0.74 μg/mL (for 4I22) eeds to be maintained in humans.

### Example 17

### 7H3 and 4I22 combination in healthy human volunteers

Preliminary safety data is available from a randomized, double-blind, placebo-controlled first-in-human study designed to assess the safety, tolerability and pharmacokinetics of single intravenous doses of the monoclonal antibodies in healthy subjects. In this study, 1 subject received placebo and 4 subjects received 1 mg/kg of 7H3. In the other arm of the study, 1 subject received placebo and 4 subjects received 0.1 mg/kg of 4I22. In each of the subsequent patient groups, 1 subject received placebo and 4 subjects received 7H3 and 4I22 simultaneously through two separate intravenous lines. The doses of 7H3 and 4I22 in combination were 1 mg/kg and 0.1 mg/kg, 5 mg/kg and 0.5 mg/kg, 20 mg/kg and 2 mg/kg, 50 mg/kg and 5 mg/kg. 7H3/4122 and the individual antibodies were well tolerated. In healthy subjects with the absence of viral target, preliminary data from the ongoing safety study of 7H3/4122 revealed that both 7H3 and 4I22 demonstrate typical IgG1 PK profiles with slow systemic clearance and long residence time (terminal elimination half-life around 21 days). The PK of both antibodies was linear with tight inter-individual variability within each cohort.

### Example 18

### Dosing Design in Humans

This example describes a design for administration to humans of a combination of 7H3 and 4I22. The actual administration to humans of this combination is described in Example 19.

Patients can receive intravenous (IV) doses of 7H3 and 4I22 sequentially (as two staggered short IV infusions). The initial dosing interval can be every 28 days but the dosing interval may be adjusted to be more frequent in order to maintain adequate trough levels of both antibodies to stay above the target efficacious levels (7.4 and 0.74 μg/mL). Once the initial PK data for at least 4 weeks after the first dose of 7H3/4I22 become available from greater than four patients in the cohort, decision can be made whether and how to adjust the dosing interval for subsequent doses. If dosing frequency remains once every 4 weeks for the entire treatment period, patients can be dosed on Day 1, Day 29, Day 57, and Day 85. Potential dosing intervals can be no more frequent than once a week and no less frequent than once every 4 weeks. The dosing days for these 4 dosing intervals are listed below.
1 week: Day 1, Day 8, Day 15, Day 22, Day 29, Day 36, Day 43, Day 50, Day 57, Day 64, Day 71, Day 78, Day 85 and Day 92
2 weeks: Day 1, Day 15, Day 29, Day 43, Day 57, Day 71 and Day 85
3 weeks: Day 1, Day 22, Day 43, Day 64 and Day 85
4 weeks: Day 1, Day 29, Day 57, and Day 85.

The initial dose of 7H3/4122 can be administered the day before the stem cell transplant conditioning regimen starts. Subsequent doses can be administered every 4 weeks unless initial PK data obtained indicates that more frequent administration is required to maintain adequate monoclonal antibody levels. 7H3 can be administered over a period of at least 2 hours while 4I22 can be administered over a period of at least 12 minutes. The infusions can be given either through separate catheters, separate lumens (from the same catheter) or the same catheter or lumen after flushing in between administration of 7H3 and 4I22.

The 7H3/4I22 combination is indicated for bone marrow transplant patients who may be immunosuppressed, so a pharmaceutical carrier of 50mg/ml sucrose and 10mg/ml human albumin as used previously in bone marrow transplant patients receiving CytoGam® can be used (DeRienzo et al. Pharmacotherapy 2000; 20:1175-8). Alternatively, the 7H3/4I22 combination is introduced into bone marrow transplant patients via a pharmaceutical carrier as described for another anti-viral antibody, Synagis®, as described in WO2003105894. In this disclosure, the pharmaceutical carrier was comprised of histidine and/or glycine, a saccharide (e.g. sucrose) and a polyol (e.g. polysorbate).

### Example 19

### Safety, Tolerability, and Pharmacokinetics in Humans

As discussed in Example 17, the safety, tolerability, and pharmacokinetics of a single intravenous dose of 7H3 or 4I22 or their combination was evaluated in healthy volunteers. The combination and the individual monoclonal antibodies were safe and well tolerated, with adverse events and laboratory abnormalities occurring sporadically with similar incidence between antibody and placebo groups and without any apparent relationship to dose. No subject who received antibody developed a hypersensitivity, infusion-related reaction or anti-drug antibodies. Following intravenous administration, both 7H3 and 4I22 demonstrated typical human IgG1 pharmacokinetic properties, with slow clearances, limited volumes of distribution, and long terminal half-lives. Pharmacokinetic parameters were linear and dose-proportional for both antibodies across the 50-fold range of doses evaluated in the study. There was no apparent impact on pharmacokinetics when the antibodies were administered alone or in combination.

Tolerability 7H3 and 4I22 were safe and well tolerated following single intravenous doses up to 50 mg/kg and 5 mg/kg, respectively. Both antibodies showed dose proportional linear pharmacokinetics, slow clearances, limited volumes of distribution, and long terminal half-lives, consistent with the values observed for other human IgG1 monoclonal antibodies in the absence of apparent target-mediated drug disposition. This was expected because 7H3 and 4I22 have intact Fc domains, and low levels of the targets for both antibodies are to be expected in healthy volunteers who have no evidence of actively replicating virus. As expected, administering the 2 antibodies in combination did not appear to impact the pharmacokinetic parameters of either individual antibody at dose levels well below the level at which saturation of the neonatal Fc receptor may become a factor influencing clearance. Jin et al. 2005. Human immunology 66:403-10.

In the human clinical study, 32 subjects were enrolled and received the following dosing ner treatment group.

| | | | | | | |
|---|---|---|---|---|---|---|
| 7H3 | 4I22 | 7H3 | 7H3 | 7H3 | 7H3 | Pooled |
| 1 | 0.1 | 1 mg/kg | 5 mg/kg | 20 mg/kg | 50 mg/kg | Placebo |
| mg/kg | mg/kg | 4I22 | 4I22 | 4I22 | 4I22 | N = 7 |
| N = 4 | N = 5 | 0.1 mg/kg | 0.5mg/kg | 2 mg/kg | 5 mg/kg | |
| | | N = 4 | N = 4 | N = 4 | N = 4 | |

Overall, 32 subjects were enrolled and 28 completed the study. Two subjects were lost to follow-up, one who received 4I22 (0.1 mg/kg) on Day 14 and one who received placebo on Day 19; both subjects were replaced. Two subjects withdrew consent, one who received 7H3 (50 mg/kg) and 4I22 (5 mg/kg) on Day 60 and one who received placebo on Day 34; neither subject was replaced. The subjects were predominantly male (65.6%) and Caucasian (93.8%); all subjects identified their ethnicity as Hispanic/Latino. The study population had a mean body mass index (BMI) of 27 kg/m2 and the mean age was 43 years. Twenty-eight of the 32 subjects (87.5%) had serological evidence of prior HCMV infection.

### Safety

There were no deaths during the study and no subject discontinued due to an adverse event. There was one serious adverse event, a Grade 1 transient ischemic attack on Day 58, which developed in a subject who received 1 mg/kg of 7H3, 57 days earlier. The subject was admitted to the hospital for observation and his symptoms resolved without treatment. The event was not considered by the investigator to be related to study drug. The subject reported no further symptoms or concomitant medications during follow-up and completed the study as planned.

Seventeen treatment-emergent adverse events occurred during the study period: 14 among 7 subjects who received antibody and 3 among 2 subjects who received placebo. The percentages of subjects who developed adverse events were similar among those administered antibody (7/25; 28.0%) or placebo (2/7; 28.6%). Adverse events occurred sporadically and without any apparent relationship to treatment group or dose.

All adverse events were assessed as Grade 1 in severity and all resolved, with only 3 events (transient ischemic attack, dizziness, and an influenza-like illness) in 2 subjects requiring action. All but 1 adverse event were assessed as not related to study drug. The 1 event suspected to be related was palpitations, which developed in a subject who received 20 mg/kg of 7H3 and 2 mg/kg of 4I22 the day before. This subject also developed myalgia and headache that same day, Day 2. All 3 events resolved after 2 hours without any action taken. The myalgia and headache were not assessed as related to study drug.

The most common adverse events were dizziness (n=3) and palpitations (n=2).

Dizziness developed in 2 subjects who received antibody and 1 subject who received placebo. The first subject received 0.1 mg/kg of 4I22, and he developed dizziness on Day 53; no action was taken. This subject had also developed palpitations on Day 28. The second subject received 1 mg/kg of 7H3 and 0.1 mg/kg of 4I22, and she developed dizziness and syncope on Day 88; the dizziness was treated with promethazine. This subject also developed an influenza-like illness on Day 87 and menorrhagia on Day 40. The third subject received placebo, and he developed Grade 1 dizziness on Day 1 shortly after completion of infusion; no action was taken. Palpitations developed in 2 subjects who received antibody; both subjects are discussed above. No treatment site or infusion-related reactions were reported, although 1 subject who received placebo developed pruritus and erythematous rash on the hands within 5 hours of dosing on Day 1; the pruritus and rash resolved without treatment. Only 1 subject, who received 5 mg/kg of 7H3 and 0.5 mg/kg of 4I22, had a laboratory abnormality assessed as an adverse event. This subject had a Grade 1 low blood glucose level on Day 105. No other laboratory abnormality was assessed as clinically significant, and no clinically significant vital sign or electrocardiogram abnormalities were noted during the study.

### Pharmacokinetics.

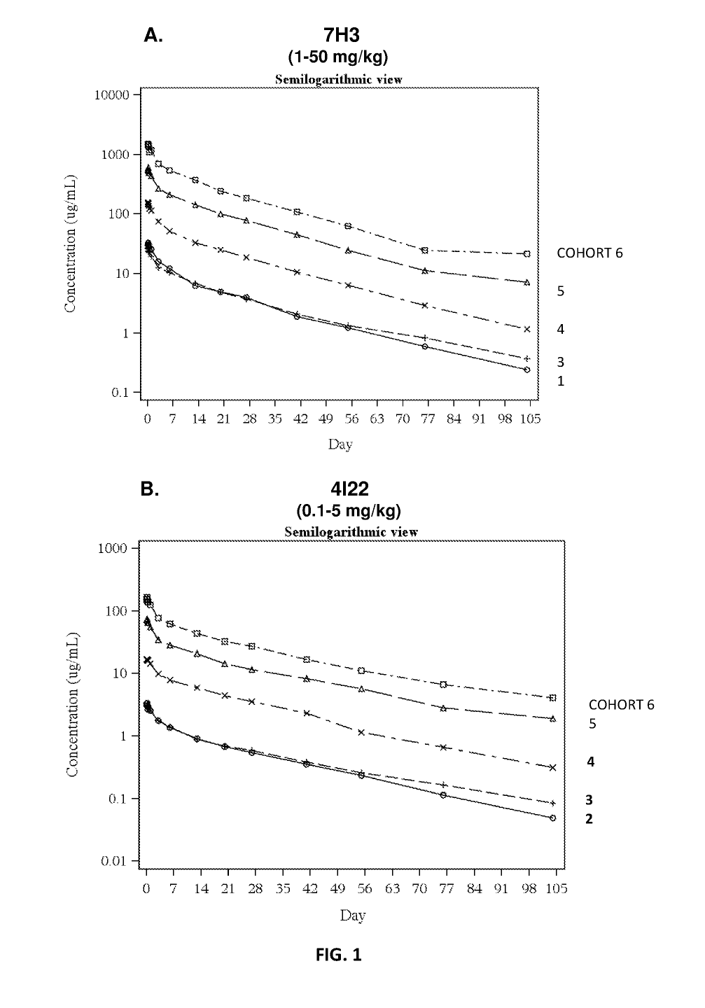

Pharmacokinetic properties were consistent within each cohort, with limited inter-individual variability noted in the serum concentration versus time profiles and in the non-compartmental pharmacokinetic parameter estimates. Following the 2-hour intravenous infusions of 7H3 and 4I22, serum concentrations of both antibodies increased rapidly and reached the maximum concentration around the end of infusion (FIG. 1). After infusion, serum concentrations decreased rapidly during the initial few days, followed by a much slower terminal elimination phase. Overall, pharmacokinetic characteristics of both 7H3 and 4I22 were typical of human IgG1 antibodies, with slow clearances, limited apparent volumes of distribution, and long terminal half-lives. Administration of the two antibodies in combination did not appear to impact the pharmacokinetics of either individual antibody, since the pharmacokinetic profiles and parameters were generally comparable between Cohorts 1 and 3 for 7H3 at 1 mg/kg and Cohorts 2 and 3 for 4I22 at 0.1 mg/kg. In addition, prior infection with HCMV based on serology had no appreciable impact on the pharmacokinetics of 7H3 or 4I22. HCMV DNA was detectable in two subjects 3 and 15 weeks after administration of 7H3 (1 mg/kg) alone and 4122 (0.1 mg/kg) alone, respectively. In both cases the amount of HCMV DNA was below the lower limit of the quantitative range (200 IU/mL).

Across the 50-fold dose ranges (1-50 mg/kg for 7H3 and 0.1-5 mg/kg for 4I22, a 10:1 ratio), key pharmacokinetic parameter estimates for serum exposure (AUC and Cmax), clearance (CL), distribution (Vss), and terminal half-life (T1/2) remained relatively constant, indicating linear pharmacokinetics across the range of doses, were evaluated. Based on the statistical analyses, dose proportionality was demonstrated for AUClast, AUCinf and Cmax across the whole range of doses for 7H3 and 4I22 (Table 18).

TABLE 18
Dose proportionality of pharmacokinetic parameters

| | | | | | |
|---|---|---|---|---|---|
| | | | Estimated | | |
| | | | increase | | Dose proportionality |
| | Slope | 90% CI | across dose | 90% CI for | across the whole |
| Parameter | estimate | for slope | range* | increase | range based on slope |

7H3

| | | | | | |
|---|---|---|---|---|---|
| AUClast | 1.00 | (0.95, 1.05) | 50.50 | (41.79, 61.03) | Yes |
| (day*μg/mL) | | | | | |
| AUCinf | 1.00 | (0.95, 1.05) | 50.72 | (41.65, 61.77) | Yes |
| (day*μg/mL) | | | | | |
| Cmax | 1.00 | (0.97, 1.03) | 50.03 | (44.52, 56.21) | Yes |
| (μg/mL) | | | | | |

4I22

| | | | | | |
|---|---|---|---|---|---|
| AUClast | 0.99 | (0.94, 1.04) | 48.48 | (40.02, 58.74) | Yes |
| (day*μg/mL) | | | | | |
| AUCinf | 1.00 | (0.95, 1.05) | 49.21 | (40.37, 60.00) | Yes |
| (day*μg/mL) | | | | | |
| Cmax | 1.02 | (0.99, 1.04) | 53.84 | (48.70, 59.52) | Yes |
| (μg/mL) | | | | | |

*Dose range was 1-50 mg/kg for LJP538 and 0.1-5 mg/kg for LJP539. Estimates and 90% CI of the exponent are determined from a simple linear regression analysis of the log-transformed values of the pharmacokinetic parameter and dose.

### Immunogenicity

Among the 25 subjects administered 7H3 and/or 4I22, no anti-drug antibodies were detected either at baseline or any of the post-dose time points (Day 14, Day 28, Day 56 and Day 105). 7H3 concentrations in 19 samples from all subjects in Cohorts 5 and 6 who received antibody exceeded the drug tolerance level (48.7 μg/mL) for the anti-drug antibody assay at the earlier time points; above the drug tolerance level there is the potential for interference with the assay and the presence of anti-drug antibodies can therefore not be fully excluded. However, samples from the same subjects at later time points, especially on Day 105, indicated that no subjects treated with either 7H3 and/or 4I22 had anti-drug antibodies by the end of the study. Moreover, the typical IgG1 pharmacokinetic profiles with the absence of accelerated clearance during the long terminal elimination phase (a common phenomenon associated with anti-drug antibody formation against therapeutic monoclonal antibodies) also suggest that anti-drug antibodies had not developed during the study. No anti-drug antibodies were detected in any of the samples from subjects who received placebo.

Unless defined otherwise, the technical and scientific terms used herein have the same meaning as that usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims are non-limiting and are provided below.

SEQ ID List

| SEQ ID | mAb | Description | Sequence |
|---|---|---|---|
| 1 | 4N10 | CDRH1 aa | GGTFSSYV |
| 2 | | CDRH2 aa | VIPIFDTV |
| 3 | | CDRH3 aa | ARGILAYCGGDCYNTPYGMDV |
| 4 | | CDRL1 aa | QSISSW |
| 5 | | CDRL2 aa | KAS |
| 6 | | CDRL3 aa | QQYNSSWT |
| 7 | | CDRH1 nuc | ggaggcaccttcagcagctatgtt |
| 8 | | CDRH2 nuc | gtcatccctatctttgatacagta |
| 9 | | CDRH3 nuc | gcgagaggaattctagcatattgtggtggtgattgctataataccccttacggtatggacgtc |
| 10 | | CDRL1 nuc | cagagtattagtagctgg |
| 11 | | CDRL2 nuc | aaggcgtct |
| 12 | | CDRL3 nuc | caacagtataatagttcgtggacg |
| 13 | | heavy ch aa | QVQLVQSGAEVKKPGSSVRVSCKASGGTFSSYVIIWVRQAPGQGLEWMGGVIPIFDTVNYAQK |
| | | | FQGRVTITADESTSTAYMELSSLKSEDTAVYYCARGILAYCGGDCYNTPYGMDVWGQGTTVTV |
| | | | SS |
| 14 | | light ch aa | DIQMTQSPSILSASVGDRVTITCRASQSISSWLAWYQQKPGKAPKLLIYKASSLEIGVPSRIS |
| | | | GSGSGTEFTLTISSLQPDDFATYYCQQYNSSWTFGQGTKVEIK |
| 15 | | heavy ch nuc | caggtgcagctggtgcagtctggggctgaggtgaagaagcctgggtcctcggtgagggtctcc |
| | | | tgcaaggcttctggaggcaccttcagcagctatgttatcatctgggtgcgacaggcccctgga |
| | | | caaggtcttgagtggatggggggggtcatccctatctttgatacagtaaattacgcacagaag |
| | | | ttccagggcagagtcacgattaccgcggacgaatccacgagtactgcctacatggagctgagc |
| | | | agcctgaaatctgaggacacggccgtatattactgtgcgagaggaattctagcatattgtggt |
| | | | ggtgattgctataataccccttacggtatggacgtctggggccaagggaccacggtcaccgtc |
| | | | tcctcag |
| 16 | | light ch nuc | gacatccagatgacccagtctccttccatcctgtctgcatctgtaggagacagagtcaccatc |
| | | | acttgccgggccagtcagagtattagtagctggttggcctggtatcagcagaaaccagggaaa |
| | | | gccccaaaactcctaatctataaggcgtctagtttagaaattggggtcccatcaaggatcagc |
| | | | ggcagtggatctgggacagaattcactctcaccatcagcagcctgcagcctgatgattttgca |
| | | | acttattactgccaacagtataatagttcgtggacgttcggccaagggacgaaggtggaaatc |
| | | | aaac |
| 17 | 10F7 | CDRH1 aa | GFTFGDYA |
| 18 | | CDRH2 aa | IRSKAYGGTT |
| 19 | | CDRH3 aa | TRASSLLWLLNPQPNFDY |
| 20 | | CDRL1 aa | NIGSNN |
| 21 | | CDRL2 aa | DDS |
| 22 | | CDRL3 aa | QVWDSSSDHPV |
| 23 | | CDRH1 nuc | ggattcacctttggtgattatgct |
| 24 | | CDRH2 nuc | attagaagcaaagcttatggtgggacaaca |
| 25 | | CDRH3 nuc | actagagcatcttcattactatggttactaaaccctcaacccaactttgactac |
| 26 | | CDRL1 nuc | aacattggaagtaacaat |
| 27 | | CDRL2 nuc | gatgatagc |
| 28 | | CDRL3 nuc | caggtgtgggatagtagtagtgatcatccggta |
| 29 | | heavy ch aa | EVQLVESGGGLVQPGRSLRLSCTASGFTFGDYAMSWFRQAPGKGLEWVGFIRSKAYGGTTEYA |
| | | | ASVKGRFTISRDDSKSIAYLQMNSLKTEDTAVYYCTRASSLLWLLNPQPNFDYWGQGTLVTVS |
| | | | S |
| 30 | | light ch aa | SYVLTQPPSVSVAPGQTARITCGGNNIGSNNVHWYQQKPGQAPVLVVYDDSDRPSGIPERFSG |
| | | | SNSGNTATLTISRVEAGDEADYYCQVWDSSSDHPVFGGGTKLTVL |
| 31 | | heavy ch nuc | gaggtgcagctggtggagtctgggggaggcttggtacagccagggcggtccctgagactctcc |
| | | | tgtacagcttctggattcacctttggtgattatgctatgagctggttccgccaggctccaggg |
| | | | aaggggctggagtgggtaggtttcattagaagcaaagcttatggtgggacaacagaatacgcc |
| | | | gcgtctgtgaaaggcagattcaccatctcaagagatgattccaaaagcatcgcctatctgcaa |
| | | | atgaacagcctgaaaaccgaggacacagccgtgtattactgtactagagcatcttcattacta |
| | | | tggttactaaaccctcaacccaactttgactactggggccagggaaccctggtcaccgtctcc |
| | | | tcag |
| 32 | | light ch nuc | tcctatgtgctgactcagccaccctcggtgtcagtggccccaggacagacggccaggattacc |
| | | | tgtgggggaaacaacattggaagtaacaatgtgcactggtaccagcagaagccaggccaggcc |
| | | | cctgtgctggtcgtctatgatgatagcgaccggccctcagggatccctgagcgattctctggc |
| | | | tccaactctgggaacacggccaccctgaccatcagcagggtcgaagccggggatgaggccgac |
| | | | tattactgtcaggtgtgggatagtagtagtgatcatccggtattcggcggagggaccaagctg |
| | | | accgtcctag |
| 33 | 10P3 | CDRH1 aa | GFTFHNYR |
| 34 | | CDRH2 aa | IKQDGSEK |
| 35 | | CDRH3 aa | ARGEGYTYGVVYSYSAMDV |
| 36 | | CDRL1 aa | VLPNQY |
| 37 | | CDRL2 aa | KDT |
| 38 | | CDRL3 aa | QSADSSGADYV |
| 39 | | CDRH1 nuc | ggattcacctttcataactatcgc |
| 40 | | CDRH2 nuc | ataaagcaagatggaagtgagaaa |
| 41 | | CDRH3 nuc | gcgaggggtgaagggtacacctatggtgtcgtctactcctattccgctatggacgtc |
| 42 | | CDRL1 nuc | gtattgccaaaccaatat |
| 43 | | CDRL2 nuc | aaagacact |
| 44 | | CDRL3 nuc | caatcagcagacagcagtggtgccgattatgtc |
| 45 | | heavy ch aa | EVQLVESGGGLVRPGGSLRLSCAASGFTFHNYRMNWVRQAPGKGLEWVANIKQDGSEKSYVDS |
| | | | VRGRFTTSRDNSKNSLYLQINSLRAEDTAVYYCARGEGYTYGVVYSYSAMDVWGQGTTVIVSS |
| 46 | | light ch aa | SYELTQPPSVSVSPGQTARITCSGNVLPNQYASWYQQKPGQAPVLVIYKDTERPSGIPGRFSG |
| | | | SSSGTTVTLTISGVQAEDEADYYCQSADSSGADYVFGTGTKVTVL |
| 47 | | heavy ch nuc | gaggtgcagctggtagagtctgggggaggcttggtccggcctggggggtccctgagactctca |
| | | | tgtgcagcctctggattcacctttcataactatcgcatgaactgggtccgccaggctccaggg |
| | | | aaggggctggagtgggtggccaacataaagcaagatggaagtgagaaatcctatgtggactct |
| | | | gtgaggggccgattcaccacctccagagacaactccaagaattcactctatctgcaaattaac |
| | | | agcctgcgagccgaggacacggctgtctattactgtgcgaggggtgaagggtacacctatggt |
| | | | gtcgtctactcctattccgctatggacgtctggggccaagggaccacagtcatcgtctcctca |
| | | | g |
| 48 | | light ch nuc | tcctatgagctgacacagccaccctcggtgtcagtgtccccaggacagacggccaggatcacc |
| | | | tgctctggaaatgtattgccaaaccaatatgcttcttggtaccagcagaagccaggccaggcc |
| | | | cctgtattggtgatatataaagacactgagaggccctcagggatccctgggcgattctctggc |
| | | | tccagctcagggacgacagtcacgttgaccatcagtggagtccaggcagaggacgaggctgac |
| | | | tattactgtcaatcagcagacagcagtggtgccgattatgtcttcggaactgggaccaaggtc |
| | | | accgtcctag |
| 49 | 4I22 | CDRH1 aa | GFTFSSYA |
| 50 | | CDRH2 aa | ISYDGDNK |
| 51 | | CDRH3 aa | AREELVGLMPPYYNYGLDV |
| 52 | | CDRL1 aa | NSNIGNNY |
| 53 | | CDRL2 aa | DND |
| 54 | | CDRL3 aa | ETWDTSLSAAVV |
| 55 | | CDRH1 nuc | ggattcaccttcagttcctatgct |
| 56 | | CDRH2 nuc | atttcatatgatggcgacaacaaa |
| 57 | | CDRH3 nuc | gcgagagaagagttagtcgggttgatgcctccctattacaactacggattggacgtc |
| 58 | | CDRL1 nuc | aactccaacatcgggaataattat |
| 59 | | CDRL2 nuc | gacaatgat |
| 60 | | CDRL3 nuc | gaaacatgggataccagcctgagtgctgctgttgtc |
| 61 | | heavy ch aa | QVQLVESGGGVVQPGRSLRLSCVASGFTFSSYAMHWVRQAPGKGLEWVAVISYDGDNKFYADS |
| | | | VKGRFRISRDTSKNTLYLEMNSLRAADTAIYYCAREELVGLMPPYYNYGLDVWGQGTTVTVSS |
| 62 | | light ch aa | QSVLTQPPSVSAAPGQKVTISCSGSNSNIGNNYVSWYQQLPGRAPKLLIYDNDHRPSGIPDRF |
| | | | SGSKSGTSATLVITGLQTGDEADYYCETWDTSLSAAVVFGGGTKLTVL |
| 63 | | heavy ch nuc | caggtgcagctggtggagtctgggggaggggtggtccagcctgggaggtccctgagactctcc |
| | | | tgtgtagcctctggattcaccttcagttcctatgctatgcactgggtccgccaggctccaggc |
| | | | aagggactggagtgggtggcagttatttcatatgatggcgacaacaaattctacgcagactcc |
| | | | gtgaagggccgattcaggatctccagagacacatccaagaatacactgtatctggaaatgaac |
| | | | agcctgagagctgcggacacggctatatattactgtgcgagagaagagttagtcgggttgatg |
| | | | cctccctattacaactacggattggacgtctggggccaaggaaccacggtcaccgtctcgtca |
| | | | g |
| 64 | | light ch nuc | cagtctgtgttgactcagccgccctcagtgtctgcggccccaggacagaaggtcaccatctcc |
| | | | tgctctggaagcaactccaacatcgggaataattatgtatcgtggtaccagcagctcccagga |
| | | | agagcccccaaactcctcatttatgacaatgatcaccgaccctcagggattcctgaccgattc |
| | | | tctggctccaagtctggcacgtcagccaccctggtcatcaccggactccagactggggacgag |
| | | | gccgattattactgcgaaacatgggataccagcctgagtgctgctgttgtcttcggcggaggg |
| | | | accaagctgaccgtcctac |
| 65 | 2C12 | CDRH1 aa | GFSLNTNGVG |
| 66 | | CDRH2 aa | IYWNGNE |
| 67 | | CDRH3 aa | VHWPQGLTTVTRLAFDI |
| 68 | | CDRL1 aa | TSDVGRYNF |
| 69 | | CDRL2 aa | DVS |
| 70 | | CDRL3 aa | CSYAGGNFFSYV |
| 71 | | CDRH1 nuc | ggcttctcactcaacactaatggagtgggt |
| 72 | | CDRH2 nuc | atttactggaatggtaatgag |
| 73 | | CDRH3 nuc | gtacactggccccaagggttgactacggtgacaagacttgcttttgatatc |
| 74 | | CDRL1 nuc | accagtgatgttggtcgttataacttt |
| 75 | | CDRL2 nuc | gatgtcagt |
| 76 | | CDRL3 nuc | tgctcatatgcaggcggcaattttttctcttatgtc |
| 77 | | heavy ch aa | QITLRESGPTLVKPTQTLTLTCTFSGFSLNTNGVGVGWIRQPPGKALEWLALIYWNGNEGYSP |
| | | | SLKSRLTITKDTSKNQVVLTMTNMDPVDTATYYCVHWPQGLTTVTRLAFDIWGQGTMVTVSS |
| 78 | | light ch aa | QSALTQPRSVSGSPGQSVTISCTGTTSDVGRYNFVSWYQQHPGKAPKLLMYDVSQRPSGVPSR |
| | | | FSGSKSGNTASLTISGLQAEDEAVFYCCSYAGGNFFSYVFGTGTKVTVL |
| 79 | | heavy ch nuc | cagatcaccttgagggagtctggtcctacgctggtgaaacccacacagaccctcacgctgacc |
| | | | tgcaccttctctggcttctcactcaacactaatggagtgggtgtgggctggatccgtcagccc |
| | | | ccaggaaaggccctggagtggcttgcactcatttactggaatggtaatgagggctacagcccc |
| | | | tctctgaaaagcagactcaccatcaccaaggacacctccaaaaaccaggtggtcctgacaatg |
| | | | accaacatggaccctgtggacacagccacatattactgtgtacactggccccaagggttgact |
| | | | acggtgacaagacttgcttttgatatctggggccaagggactatggtcaccgtctcttcag |
| 80 | | light ch nuc | cagtctgccctgactcagcctcgctcagtgtccgggtctcctggacagtcagtcaccatctcc |
| | | | tgcactggaaccaccagtgatgttggtcgttataactttgtctcctggtaccaacaacaccca |
| | | | ggcaaagcccccaaactcctgatgtatgatgtcagtcagcggccctcaggggtccctagtcgc |
| | | | ttctctggctccaagtctggcaacacggcctccctgaccatctctgggctccaggctgaggat |
| | | | gaggctgttttttactgctgctcatatgcaggcggcaattttttctcttatgtcttcggaact |
| | | | gggaccaaggtcaccgtcctag |
| 81 | 8C15 | CDRH1 aa | GGSIRSYY |
| 82 | | CDRH2 aa | IYYSGNT |
| 83 | | CDRH3 aa | ARHDVIVVRGVFDV |
| 84 | | CDRL1 aa | SSDIGTYNL |
| 85 | | CDRL2 aa | DGS |
| 86 | | CDRL3 aa | CSYAGTSDFFVV |
| 87 | | CDRH1 nuc | ggtggctccatccggagttactac |
| 88 | | CDRH2 nuc | atctattacagtgggaacacc |
| 89 | | CDRH3 nuc | gcgagacatgatgtgatagtagtccgcggtgtctttgatgtc |
| 90 | | CDRL1 nuc | agcagtgatattggaacttataacctt |
| 91 | | CDRL2 nuc | gatggcagt |
| 92 | | CDRL3 nuc | tgctcatatgctggtactagcgatttctttgtggtt |
| 93 | | heavy ch aa | QVQLQESGPGLVKPSETLSLTCTVSGGSIRSYYWSWIRQPPGKGLEWIGHIYYSGNTNYSPSL |
| | | | QSRVTISLDTPKNQFSLRLSSVTAADTAVYYCARHDVIVVRGVFDVWGQGTVVTVSS |
| 94 | | light ch aa | QSALTQPASVSGSPGQSITISCTGTSSDIGTYNLVSWYQQHPGKAPKVLIYDGSKRPSGVSSR |
| | | | FSASKSGNTASLTISGLQAEDETDYYCCSYAGTSDFFVVFGGGTKLTVL |
| 95 | | heavy ch nuc | caggtgcagctgcaggagtcgggcccaggtctggtgaagccttcggagaccctgtccctcacc |
| | | | tgcactgtctctggtggctccatccggagttactactggagctggatccggcagcccccaggg |
| | | | aagggactggagtggattgggcacatctattacagtgggaacaccaactacagcccctccctc |
| | | | cagagtcgagtcaccatatcattagacacgcccaagaaccaattctccctgcggctgagctct |
| | | | gtgaccgccgcagacacggccgtctattactgtgcgagacatgatgtgatagtagtccgcggt |
| | | | gtctttgatgtctggggccaagggacagtggtcaccgtctcttcag |
| 96 | | light ch nuc | cagtctgccctgactcagcctgcctccgtgtctgggtcacctggacagtcgatcaccatctcc |
| | | | tgcactggaaccagcagtgatattggaacttataaccttgtctcctggtaccaacaacaccca |
| | | | ggcaaagcccccaaagtcctaatttatgatggcagtaagcggccctcaggggtttctagtcgc |
| | | | ttctctgcctccaagtctggcaacacggcctccctgacaatctctgggctccaggctgaggac |
| | | | gagactgattattactgctgctcatatgctggtactagcgatttctttgtggttttcggcgga |
| | | | gggaccaagctgaccgtcctgg |
| 97 | 9I6 | CDRH1 aa | GDTFPAYW |
| 98 | | CDRH2 aa | IYPIDSET |
| 99 | | CDRH3 aa | ARGTSTGLREAFHI |
| 100 | | CDRL1 aa | QSLGYSDGNTY |
| 101 | | CDRL2 aa | EVS |
| 102 | | CDRL3 aa | MQGTHWPPMCS |
| 103 | | CDRH1 nuc | ggagacacttttcccgcctactgg |
| 104 | | CDRH2 nuc | atctatcctattgactctgagacc |
| 105 | | CDRH3 nuc | gcccgggggacaagtactggcctcagagaggcttttcatatc |
| 106 | | CDRL1 nuc | caaagcctcggatacagtgatggaaacacctat |
| 107 | | CDRL2 nuc | gaggtttct |
| 108 | | CDRL3 nuc | atgcaaggtacacactggcctcccatgtgcagt |
| 109 | | heavy ch aa | EVQLVQSGAEVKKPGESLKISCRESGDTFPAYWIAWVRQMPGKGLEWMGIIYPIDSETTYSPS |
| | | | FQGQVTISADKSINTAYLQWSSLKASDSAIYYCARGTSTGLREAFHIWGQGTMVTVSS |
| 110 | | light ch aa | DVVMTQSPLSLAVTLGQPAYISCRSSQSLGYSDGNTYLNWFQQRPGQSPRRLIYEVSNRDSGV |
| | | | PDRFSGSGSGTDFTLKISRVEAEDVGTYYCMQGTHWPPMCSFGQGTKLEIK |
| 111 | | heavy ch nuc | gaggtgcagctggtgcagtctggagcagaggtgaaaaagcccggggagtctctgaagatctcc |
| | | | tgtagggaatctggagacacttttcccgcctactggatcgcctgggtgcgccagatgcccggg |
| | | | aaaggcctggagtggatgggaattatctatcctattgactctgagaccacatatagcccgtcc |
| | | | ttccaaggccaggtcaccatttcagccgacaagtccatcaacaccgcctacctgcagtggagc |
| | | | agcctgaaggcctcggactccgccatttattactgtgcccgggggacaagtactggcctcaga |
| | | | gaggcttttcatatctggggccaagggacaatggtcaccgtctcttcag |
| 112 | | light ch nuc | gatgttgtgatgactcagtctccactctccctggccgtcacccttggacagccggcctacatc |
| | | | tcctgcaggtcaagtcaaagcctcggatacagtgatggaaacacctatttgaattggtttcag |
| | | | cagagaccaggccaatctcccaggcgcctaatttatgaggtttctaaccgggactctggggtc |
| | | | ccagacagattcagcggcagtgggtcgggcactgatttcacactgaaaatcagcagggtggag |
| | | | gctgaggatgttgggacttattactgcatgcaaggtacacactggcctcccatgtgcagtttt |
| | | | ggccaggggaccaagttggagatcaaac |
| 113 | 8L13 | CDRH1 aa | GFTFSNYG |
| 114 | | CDRH2 aa | IWNDGSKK |
| 115 | | CDRH3 aa | ARDEGVQMVFAMPDYGMDV |
| 116 | | CDRL1 aa | KLGDKF |
| 117 | | CDRL2 aa | QDS |
| 118 | | CDRL3 aa | QAWDSSTAHYV |
| 119 | | CDRH1 nuc | ggattcaccttcagtaattatggc |
| 120 | | CDRH2 nuc | atatggaatgatggaagtaagaaa |
| 121 | | CDRH3 nuc | gcgagagatgaaggtgtacaaatggtgttcgccatgcctgactacggtatggacgtc |
| 122 | | CDRL1 nuc | aaattgggggataaattc |
| 123 | | CDRL2 nuc | caagattcc |
| 124 | | CDRL3 nuc | caggcgtgggacagcagcactgcccattatgtc |
| 125 | | heavy ch aa | QVQLLESGGGVVQPGRSLRLSCAASGFTFSNYGMHWVRQAPGKGLEWVAVIWNDGSKKYYAES |
| | | | VKGRFTISRDNSKNTVYLQMNSLRAEDTAVYYCARDEGVQMVFAMPDYGMDVWGQGTTVTVSS |
| 126 | | light ch aa | SYELTQPPSVSVSPGQTASITCSGDKLGDKFACWYQQRPGQSPILVIYQDSKRPSGIPERFSG |
| | | | SNSGNTATLTIRGTQAMDEADYYCQAWDSSTAHYVFGTGTKVTVL |
| 127 | | heavy ch nuc | caggtgcagttgctggagtctgggggaggcgtggtccagcctgggaggtccctgagactctcc |
| | | | tgtgcagcgtctggattcaccttcagtaattatggcatgcactgggtccgccaggctccaggc |
| | | | aaggggctggagtgggtggcagttatatggaatgatggaagtaagaaatattatgcagagtcc |
| | | | gtgaagggccggttcaccatctccagagacaattccaagaacacagtatatctacaaatgaac |
| | | | agcctgagagccgaggacacggctgtgtattactgtgcgagagatgaaggtgtacaaatggtg |
| | | | ttcgccatgcctgactacggtatggacgtctggggccaggggaccacggtcaccgtctcctca |
| | | | g |
| 128 | | light ch nuc | tcctatgaactgactcagccaccctcagtgtccgtgtccccaggacagacagccagcatcact |
| | | | tgctctggagataaattgggggataaattcgcttgctggtatcagcagaggccaggccagtct |
| | | | cctatactggtcatctatcaagattccaagcggccctcagggatccctgagcgattctctggc |
| | | | tccaactctgggaacacagccactctgaccatccgcgggacccaggctatggatgaggctgac |
| | | | tattactgtcaggcgtgggacagcagcactgcccattatgtcttcggaactgggaccaaggtc |
| | | | accgtccttg |
| 129 | 7B13 | CDRH1 aa | GFSFSNYG |
| 130 | | CDRH2 aa | IPSDGNYQ |
| 131 | | CDRH3 aa | AHLGGGLFDF |
| 132 | | CDRL1 aa | SSDVGGYEF |
| 133 | | CDRL2 aa | DVD |
| 134 | | CDRL3 aa | YSSADTWV |
| 135 | | CDRH1 nuc | ggattctccttcagtaattatggc |
| 136 | | CDRH2 nuc | ataccgtctgatggaaattatcaa |
| 137 | | CDRH3 nuc | gcccacctcggggggggtttatttgacttc |
| 138 | | CDRL1 nuc | agcagtgatgttggtggttatgagttt |
| 139 | | CDRL2 nuc | gatgtcgat |
| 140 | | CDRL3 nuc | tactcatctgcagacacctgggtc |
| 141 | | heavy ch aa | QVQLVESGGGVVQPGGSLRLSCAASGFSFSNYGMHWVRQAPGKGLEWVALIPSDGNYQYYTDS |
| | | | VKGRFTVSRDNSRNTLYLQMKSLRAEDTARYHCAHLGGGLFDFWGQGTLVTVSS |
| 142 | | light ch aa | QSALNQPRSVSGSPGQSVSISCTGSSSDVGGYEFVSWYQHHPGKAPKLIIYDVDKRPSGVPDR |
| | | | FSGSRSGDTASLTISGLQAEDEADYYCYSSADTWVFGGGTKLTVL |
| 143 | | heavy ch nuc | caggtgcagctggtggagtctgggggaggcgtggtccagcctggggggtccctgagattgtcc |
| | | | tgtgcagcgtctggattctccttcagtaattatggcatgcactgggtccgccaggctccaggc |
| | | | aaggggctggagtgggtggcacttataccgtctgatggaaattatcaatactatacagactcc |
| | | | gtgaagggccgattcaccgtctccagagacaattccaggaacacgttgtatctgcaaatgaag |
| | | | agcctgagagctgaggacacggctagatatcattgtgcccacctcggggggggtttatttgac |
| | | | ttctggggccagggcaccctggtcaccgtctcctcag |
| 144 | | light ch nuc | cagtctgccctgaatcagcctcgctcagtgtccgggtctcctggacagtcagtctccatctcc |
| | | | tgcactggctccagcagtgatgttggtggttatgagtttgtctcctggtaccaacaccaccca |
| | | | ggcaaagcccccaaactcataatttatgatgtcgataagcggccctcaggggtccctgatcgc |
| | | | ttctctggctccaggtctggcgacacggcctccctgaccatctctgggctccaggctgaggat |
| | | | gaggctgattattactgctactcatctgcagacacctgggtcttcggcggagggaccaagctc |
| | | | actgtcctag |
| 145 | 8J16 | CDRH1 aa | GGFTSSYY |
| 146 | | CDRH2 aa | VYYGEST |
| 147 | | CDRH3 aa | AREVDKRGFDY |
| 148 | | CDRL1 aa | QSVSGGY |
| 149 | | CDRL2 aa | GAS |
| 150 | | CDRL3 aa | QQYGRTPLT |
| 151 | | CDRH1 nuc | ggtggcttcaccagtagttattat |
| 152 | | CDRH2 nuc | gtgtattacggtgaaagtacc |
| 153 | | CDRH3 nuc | gcgagagaagtggataaacggggctttgactac |
| 154 | | CDRL1 nuc | cagagtgttagcggcggttac |
| 155 | | CDRL2 nuc | ggtgcatcc |
| 156 | | CDRL3 nuc | cagcagtatggtaggacaccgctcact |
| 157 | | heavy ch aa | QVQLQESGPGLVKPSETLSLTCSVSGGFTSSYYWSWIRQAPGKGLEWIGYVYYGESTDYNPSL |
| | | | KSRATISIDTSKNQFSLKLSSVTAADTAVYYCAREVDKRGFDYWGQGALVTVSS |
| 158 | | light ch aa | EIVLTQSPGTLSLSPGERATLSCRASQSVSGGYLAWYQQEPGQAPRLVIYGASSRATGIPDRF |
| | | | SASGSGTDFTLTITRLEPEDFAVYYCQQYGRTPLTFGGGTKVEIK |
| 159 | | heavy ch nuc | caggtgcagctgcaggagtcgggcccaggactggtgaagccttcggagaccctgtccctcacc |
| | | | tgcagtgtctctggtggcttcaccagtagttattattggagttggatccggcaggcccccggg |
| | | | aagggactggagtggattggctatgtgtattacggtgaaagtaccgattacaacccctccctc |
| | | | aagagtcgagccaccatatcaatagacacgtccaagaaccaattctccctgaagctgagctct |
| | | | gtgaccgctgcggacacggccgtctattattgtgcgagagaagtggataaacggggctttgac |
| | | | tactggggccagggagccctggtcaccgtctcctcag |
| 160 | | light ch nuc | gaaattgtgttgacgcagtctccaggcaccctatctttgtctccaggggaaagagccaccctc |
| | | | tcctgcagggccagtcagagtgttagcggcggttacttagcctggtaccagcaggaacctggc |
| | | | caggctcccaggctcgtcatctatggtgcatccagcagggccactggcatcccagacaggttc |
| | | | agtgccagtgggtctgggacagacttcactctcaccatcaccagactggagccagaagatttt |
| | | | gcagtgtattactgtcagcagtatggtaggacaccgctcactttcggcggagggaccaaggtg |
| | | | gagatcaaac |
| 161 | 7I13 | CDRH2 aa | ISYDASSK |
| 162 | | CDRH3 aa | AKALRYLDWFLSDPFDY |
| 163 | | CDRL1 aa | QSVSSDF |
| 164 | | CDRL3 aa | QQYAASPP |
| 165 | | CDRH1 nuc | ggattcaccttcagtaactatggc |
| 166 | | CDRH2 nuc | atatcttatgatgcaagtagtaaa |
| 167 | | CDRH3 nuc | gcgaaagccctacgatatcttgactggttcctctcggaccccttcgactac |
| 168 | | CDRL1 nuc | cagagtgttagtagcgacttc |
| 169 | | CDRL3 nuc | cagcagtatgctgcctcaccgccc |
| 170 | | heavy ch aa | QVQLVESGGGVVQPGRSLRLSCAASGFTFSNYGMHWVRQGPGKGLEWVAVISYDASSKYYTDS |
| | | | VQGRFTISRDNSKNTLFLQMNSLRGEDTAVYYCAKALRYLDWFLSDPFDYWGQGTLVTVSS |
| 171 | | light ch aa | EIVLTQSPGTLSLSPGERATLSCRASQSVSSDFLAWYQQKPGQAPRLLIYGASSRATGIPDRF |
| | | | SGSGSGTDFTLTISRLEPEDFAVYYCQQYAASPPFGQGTRLEIK |
| 172 | | heavy ch nuc | caggtgcaactggtggagtctgggggaggcgtggtccagcctgggaggtccctcagactctcc |
| | | | tgtgcagcctctggattcaccttcagtaactatggcatgcactgggtccgccagggtccaggc |
| | | | aaggggctggagtgggtggcagttatatcttatgatgcaagtagtaaatactatacagactcc |
| | | | gtgcagggccgattcaccatctccagagacaattccaagaacacactgtttctgcaaatgaac |
| | | | agcctgagaggtgaagacacggctgtgtattactgtgcgaaagccctacgatatcttgactgg |
| | | | ttcctctcggaccccttcgactactggggccagggaaccctggtcaccgtctcctcag |
| 173 | | light ch nuc | gaaattgtgttgacgcagtctccaggcaccctgtctttgtctccaggggaaagagccaccctc |
| | | | tcctgcagggccagtcagagtgttagtagcgacttcttagcctggtaccagcagaaacctggc |
| | | | caggctcccaggctcctcatctatggtgcatccagcagggccactggcatcccagacaggttc |
| | | | agtggcagtgggtctgggacagacttcactctcaccatcagccgactggagcctgaagatttt |
| | | | gcagtctattactgtcagcagtatgctgcctcaccgcccttcggccaagggacacgactggag |
| | | | attaaac |
| 174 | 8I21 | CDRH1 aa | GFTFSSDG |
| 175 | | CDRH2 aa | ISSDGSTP |
| 176 | | CDRH3 aa | AKDWALFRWLRTFDH |
| 177 | | CDRL1 aa | QSVGIN |
| 178 | | CDRL3 aa | QQYNDWPPWT |
| 179 | | CDRH1 nuc | ggattcaccttcagtagcgacggc |
| 180 | | CDRH2 nuc | atatcatctgacggaagtactcca |
| 181 | | CDRH3 nuc | gccaaagattgggcattatttcggtggctacgaacctttgatcat |
| 182 | | CDRL1 nuc | cagagtgttggcatcaat |
| 183 | | CDRL3 nuc | caacaatataatgactggcctccgtggacg |
| 184 | | heavy ch aa | LVELVESGGGVVQPGRSLRLSCAASGFTFSSDGMHWVRQSPGRGLEWVAFISSDGSTPYYADS |
| | | | VKGRFTISRDNSKNTLYLQMNSLRAEDTAMYFCAKDWALFRWLRTFDHWGQGTLVTVSS |
| 185 | | light ch aa | ETVMTQSPATLSVSPGGRATLSCRASQSVGINLAWYQQKPGQAPRLLIYGASTRASGFPARFS |
| | | | GSGSGTEFTLTITSLQSEDFAVYYCQQYNDWPPWTFGQGTKVEIK |
| 186 | | heavy ch nuc | ctggtggaactggtggagtctgggggaggcgtggtccagcctgggaggtccctgagactctcc |
| | | | tgtgcagcctctggattcaccttcagtagcgacggcatgcactgggtccgccagagtccaggc |
| | | | agggggctggaatgggtggcctttatatcatctgacggaagtactccatactatgctgactcc |
| | | | gtgaagggccgattcaccatctccagagacaattccaagaacacactgtatctgcaaatgaac |
| | | | agcctcagagctgaggacacggctatgtacttctgtgccaaagattgggcattatttcggtgg |
| | | | ctacgaacctttgatcattggggccagggaaccctggtcaccgtctcctcag |
| 187 | | light ch nuc | gaaacggtgatgacgcagtctccagccaccctgtctgtgtctcctgggggaagagccaccctc |
| | | | tcctgcagggccagtcagagtgttggcatcaatttagcctggtaccagcagaaacctggccag |
| | | | gctcccaggctcctcatctatggtgcatccaccagggcctctggtttcccagccaggttcagt |
| | | | ggcagtgggtctgggacagagttcactctcaccatcaccagcctgcagtctgaagattttgca |
| | | | gtctattactgtcaacaatataatgactggcctccgtggacgttcggccaagggaccaaggtg |
| | | | gagatcaaac |
| 188 | 15D8 | CDRH1 aa | GYSFTNYW |
| 189 | | CDRH2 aa | IYPGDSDI |
| 190 | | CDRH3 aa | ARHAIRGDGFDY |
| 191 | | CDRL1 aa | KLGEKY |
| 192 | | CDRL2 aa | QDT |
| 193 | | CDRL3 aa | QAWDTNTVI |
| 194 | | CDRH1 nuc | ggatacagctttaccaactactgg |
| 195 | | CDRH2 nuc | atctatcctggtgactctgatatc |
| 196 | | CDRH3 nuc | gcgagacatgcaatacgaggagatgggtttgactac |
| 197 | | CDRL1 nuc | aaattgggggaaaaatac |
| 198 | | CDRL2 nuc | caagatacg |
| 199 | | CDRL3 nuc | caggcgtgggacaccaacactgtgata |
| 200 | | heavy ch aa | EVQLVQSGAEVKKPGESLKISCQASGYSFTNYWIAWVRQMPGKGLEWMGIIYPGDSDIKYSPS |
| | | | FRGQVTISADKSISNAFLQWRSLRASDTAMYYCARHAIRGDGFDYWGQGTLVTVSS |
| 201 | | light ch aa | SYELTQPPSVSVSPGQTATITCSGDKLGEKYACWYQQKPGQSPVLVMYQDTKRPSGIPERFSG |
| | | | SNSGNTATLTISGTRAMDEADYYCQAWDTNTVIFGGGTKLTVL |
| 202 | | heavy ch nuc | gaggtgcagctggtgcagtctggagcagaagtgaaaaagcccggggagtctctgaagatctcc |
| | | | tgtcaggcttctggatacagctttaccaactactggatcgcctgggtgcgccagatgcccggg |
| | | | aaaggcctggagtggatgggcatcatctatcctggtgactctgatatcaaatacagcccgtcc |
| | | | ttccgaggccaggtcaccatctcagccgacaagtccatcagtaatgccttcctccagtggcga |
| | | | agcctgagggcctcggacaccgccatgtattactgtgcgagacatgcaatacgaggagatggg |
| | | | tttgactactggggccagggaaccctggtcaccgtctcctcag |
| 203 | | light ch nuc | tcctatgagctgactcagccaccctcagtgtccgtgtccccaggacagacagccaccatcacc |
| | | | tgctctggagataaattgggggaaaaatacgcttgctggtatcagcagaagccaggccagtcc |
| | | | cctgttttggtcatgtatcaagatacgaagcggccctcagggatccctgagcgattctctggc |
| | | | tccaactctgggaacacagccactctgaccatcagcgggacccgggctatggatgaagctgac |
| | | | tattactgtcaggcgtgggacaccaacactgtgatattcggcggagggaccaagctgaccgtc |
| | | | ctag |
| 204 | | CDRH2 aa var1 | IYPGDSDT |
| 205 | | CDRH3 aa var 1 | GRHAIRGDGFDY |
| 206 | | CDRH2 nuc var 1 | atctatcctggtgactctgatacc |
| 207 | | CDRH3 nuc var 2 | gggagacatgcaatacgaggagatgggtttgactac |
| 208 | | heavy ch aa var 1 | EVQLVQSGAEVKKPGESLKISCQASGYSFTNYWIAWVRQMPGKGLEWMGIIYPGDSDTKYSPS |
| | | | FRGQVTISADKSISTAFLQWRSLRASDTAMYYCGRHAIRGDGFDYWGQGTLVTVSS |
| 209 | | heavy ch nuc var 1 | gaggtgcagctggtgcagtctggagcagaagtgaaaaagcccggggagtctctgaagatctcc |
| | | | tgtcaggcttctggatacagctttaccaactactggatcgcctgggtgcgccagatgcccggg |
| | | | aaaggcctggagtggatgggcatcatctatcctggtgactctgataccaaatacagcccgtcc |
| | | | ttccgaggccaggtcaccatctcagccgacaagtccatcagtactgccttcctccagtggcga |
| | | | agcctgagggcctcggacaccgccatgtattactgtgggagacatgcaatacgaggagatggg |
| | | | tttgactactggggccagggaaccctggtcaccgtctcctcag |
| 210 | | CDRH3 aa var 2 | ERHAIRGDGFDY |
| 211 | | CDRH3 nuc var 2 | gagagacatgcaatacgaggagatgggtttgactac |
| 212 | | heavy ch aa var 2 | EVQLVQSGAEVKKPGESLKISCQASGYSFTNYWIAWVRQMPGKGLEWMGIIYPGDSDTKYSPS |
| | | | FRGQVTISADKSISTAFLQWRSLRASDTAMYYCERHAIRGDGFDYWGQGTLVTVSS |
| 213 | | light ch aa var 2 | SYVLTQPPSVSVSPGQTATITCSGDKLGEKYACWYQQKPGQSPVLVMYQDTKRPSGIPERFSG |
| | | | SNSGNTATLTISGTRAMDEADYYCQAWDTNTVIFGGGTKLTVL |
| 214 | | heavy ch nuc var 2 | gaggtgcagctggtgcagtctggagcagaagtgaaaaagcccggggagtctctgaagatctcc |
| | | | tgtcaggcttctggatacagctttaccaactactggatcgcctgggtgcgccagatgcccggg |
| | | | aaaggcctggagtggatgggcatcatctatcctggtgactctgataccaaatacagcccgtcc |
| | | | ttccgaggccaggtcaccatctcagccgacaagtccatcagtactgccttcctccagtggcga |
| | | | agcctgagggcctcggacaccgccatgtattactgtgagagacatgcaatacgaggagatggg |
| | | | tttgactactggggccagggaaccctggtcaccgtctcctcag |
| 215 | | light ch nuc var 2 | tcctatgtcctgactcagccaccctcagtgtccgtgtccccaggacagacagccaccatcacc |
| | | | tgctctggagataaattgggggaaaaatacgcttgctggtatcagcagaagccaggccagtcc |
| | | | cctgttttggtcatgtatcaagatacgaagcggccctcagggatccctgagcgattctctggc |
| | | | tccaactctgggaacacagccactctgaccatcagcgggacccgggctatggatgaagctgac |
| | | | tattactgtcaggcgtgggacaccaacactgtgatattcggcggagggaccaagctgaccgtc |
| | | | ctag |
| 216 | 13H11 | CDRH1 aa | GYTFTNYY |
| 217 | | CDRH2 aa | IHPSSGGT |
| 218 | | CDRH3 aa | GRAFRILGLSDVFVND |
| 219 | | CDRL1 aa | QGINNY |
| 220 | | CDRL2 aa | AAS |
| 221 | | CDRL3 aa | QKYNSAPFT |
| 222 | | CDRH1 nuc | ggatacaccttcaccaactactat |
| 223 | | CDRH2 nuc | atccaccctagtagtggtggcaca |
| 224 | | CDRH3 nuc | gggagagcctttcggatcttgggactttcggatgtctttgttaatgac |
| 225 | | CDRL1 nuc | cagggcattaacaattat |
| 226 | | CDRL2 nuc | gctgcatcc |
| 227 | | CDRL3 nuc | caaaagtataacagtgcccccttcact |
| 228 | | heavy ch aa | QVQLVQSGAEVKKPGASVKVSCKASGYTFTNYYIHWVRQAPGQGLEWMGIIHPSSGGTSYAQK |
| | | | FQGRVTMTRDTSTSTVSMDLSSLRSEDTAVYYCGRAFRILGLSDVFVNDWGQGTVVTVSS |
| 229 | | light ch aa | DIQMTQSPSSLSASVGDRVTITCRASQGINNYLAWYQQKPGKVPKLLIYAASTLQSGVPSRFS |
| | | | GSGSGTAFTLTILSLQPEDVATYYCQKYNSAPFTFGPGTKVDIK |
| 230 | | heavy ch nuc | caggtgcagttggtgcagtctggggctgaggtgaagaagcctggggcctcagtgaaggtttcc |
| | | | tgcaaggcatctggatacaccttcaccaactactatatacactgggtgcgacaggcccctgga |
| | | | caaggacttgagtggatgggaataatccaccctagtagtggtggcacaagctacgcacagaag |
| | | | ttccagggcagagtcaccatgaccagggacacgtccacgagcacagtttccatggacctgagc |
| | | | agcctgagatctgaagacacggccgtatattactgtgggagagcctttcggatcttgggactt |
| | | | tcggatgtctttgttaatgactggggccagggaactgtggtcaccgtctcctcag |
| 231 | | light ch nuc | gacatccagatgacccagtctccatcctccctgtctgcatctgtaggagacagagtcaccatc |
| | | | acttgccgggcgagtcagggcattaacaattatttagcctggtatcagcagaaaccagggaaa |
| | | | gttcctaagctcctgatctatgctgcatccacattgcaatcaggggtcccatctcggttcagt |
| | | | ggcagtggatctgggacagctttcaccctcaccatcctcagcctgcagcctgaagatgttgca |
| | | | acttattactgtcaaaagtataacagtgcccccttcactttcggccctgggaccaaagtggac |
| | | | atcaaac |
| 232 | 11B12 | CDRH1 aa | GFTFTSSA |
| 233 | | CDRH2 aa | IVLGSGNT |
| 234 | | CDRH3 aa | AADRGRGGYNVYTY |
| 235 | | CDRL1 aa | QTISNTY |
| 236 | | CDRL3 aa | QQNGQSPWT |
| 237 | | CDRH1 nuc | ggattcacctttactagctctgct |
| 238 | | CDRH2 nuc | atcgtccttggcagcggtaacaca |
| 239 | | CDRH3 nuc | gcggcagataggggtagaggtggatacaatgtatacacttac |
| 240 | | CDRL1 nuc | cagactattagtaacacctac |
| 241 | | CDRL3 nuc | cagcagaatggtcagtcaccttggacg |
| 242 | | heavy ch aa | QMQLVQSGPQVKKPGTSVKVSCKASGFTFTSSAMQWVRQARGQRPEWIGWIVLGSGNTNYAQK |
| | | | FQERVTLTRDMSTATAYMELSSLRSEDTAVYYCAADRGRGGYNVYTYWGQGTLVAVSS |
| 243 | | light ch aa | EIVMTQSPGTLSLSPGERATLSCRASQTISNTYVAWYQQKPGQAPRLLIYGASSRATGIPDRF |
| | | | SGSGSGTDFTLTIRRLEPEDFAVYYCQQNGQSPWTFGQGTNVEIK |
| 244 | | heavy ch nuc | caaatgcagctggtgcagtctgggcctcaagtgaagaagcctgggacctcagtgaaggtctcc |
| | | | tgcaaggcttctggattcacctttactagctctgctatgcagtgggtgcggcaggctcgtgga |
| | | | cagcgccctgagtggataggatggatcgtccttggcagcggtaacacaaactacgcacagaag |
| | | | ttccaggaaagagtcacccttaccagggacatgtccactgctacagcctacatggaactgagc |
| | | | agcctgagatccgaggacacggccgtgtattactgtgcggcagataggggtagaggtggatac |
| | | | aatgtatacacttactggggccaggggaccctggtcgccgtctcctcag |
| 245 | | light ch nuc | gaaattgtgatgacgcagtctccaggcaccctgtctttgtctccaggggaaagagccaccctc |
| | | | tcctgcagggccagtcagactattagtaacacctacgtggcctggtaccagcagaaacctggc |
| | | | caggctcccaggctcctcatctatggtgcatccagcagggccactggcatcccagacaggttc |
| | | | agtggcagtgggtctgggacagacttcactctcaccatccgcagactggagcctgaagatttt |
| | | | gcagtgtattactgtcagcagaatggtcagtcaccttggacgttcggccaagggaccaacgtg |
| | | | gaaatcaaac |
| 246 | 3G16 | CDRH1 aa | GYTFTGYY |
| 247 | | CDRH2 aa | INPMTGAT |
| 248 | | CDRH3 aa | ARGGPTSTRITGKRHFDL |
| 249 | | CDRL1 aa | ISDVGAYNS |
| 250 | | CDRL2 aa | DVT |
| 251 | | CDRL3 aa | SSYTTSDTYV |
| 252 | | CDRH1 nuc | ggatacaccttcaccggctactat |
| 253 | | CDRH2 nuc | atcaaccctatgactggagccaca |
| 254 | | CDRH3 nuc | gcgagaggaggtcctaccagtacccgaataacagggaaacggcacttcgatctc |
| 255 | | CDRL1 nuc | atcagtgacgttggtgcttataactct |
| 256 | | CDRL2 nuc | gacgtcact |
| 257 | | CDRL3 nuc | agctcatatacaaccagtgacacttatgtc |
| 258 | | heavy ch aa | RAQLVQSAAEMKNPGASVKVSCEASGYTFTGYYVHWMRQAPGQGLEWMGWINPMTGATKSPQK |
| | | | FQGRVTMTRDTSTTATHIELTRLRSDDSAVFFCARGGPTSTRITGKRHFDLWGRGTLITVAS |
| 259 | | light ch aa | QSALTQPASVSGSPGESITISCTGTISDVGAYNSVSWYQQHSGTAPELIIYDVTNRPAGVSSR |
| | | | FSGSKSGNTASLTISWLQSEDEAEYYCSSYTTSDTYVFGSGTQVTVL |
| 260 | | heavy ch nuc | cgggcgcagttggtgcagtctgcggctgagatgaagaaccctggggcctcagtgaaggtctcc |
| | | | tgcgaggcttctggatacaccttcaccggctactatgtacactggatgcgacaggcccccgga |
| | | | caaggactagagtggatgggatggatcaaccctatgactggagccacaaagtctccacagaag |
| | | | tttcagggcagggtcaccatgaccagggacacttccaccaccgcaacccacatagaactgact |
| | | | aggctgagatctgacgacagtgccgtctttttctgtgcgagaggaggtcctaccagtacccga |
| | | | ataacagggaaacggcacttcgatctctggggccgcggcaccctgatcactgtcgcctcag |
| 261 | | light ch nuc | cagtctgccctgactcagcctgcctccgtgtctgggtctcctggagagtcgatcaccatctcc |
| | | | tgcactggaaccatcagtgacgttggtgcttataactctgtctcctggtaccaacaacactca |
| | | | ggcacagcccccgaactcatcatttatgacgtcactaatcggcccgcaggggtttcgagtcgc |
| | | | ttctctggctccaagtctggcaacacggcctccctgaccatctcttggctccagtctgaggac |
| | | | gaggctgaatattattgcagctcatatacaaccagtgacacttatgtcttcggaagtgggacc |
| | | | caagtcaccgtcctaa |
| 262 | 6L3 | CDRH1 aa | GFTVSTTY |
| 263 | | CDRH2 aa | IHTGGIFGVGGT |
| 264 | | CDRH3 aa | AREHRGTIDAFDA |
| 265 | | CDRL1 aa | QNIRNY |
| 266 | | CDRL2 aa | TTS |
| 267 | | CDRL3 aa | QQSYDGWT |
| 268 | | CDRH1 nuc | ggattcaccgtcagtaccacctac |
| 269 | | CDRH2 nuc | attcataccggtggcatttttggcgttggcggtaca |
| 270 | | CDRH3 nuc | gcgagggaacatcggggaactatcgatgcttttgatgcc |
| 271 | | CDRL1 nuc | cagaacattcgaaattat |
| 272 | | CDRL2 nuc | actacatcc |
| 273 | | CDRL3 nuc | caacagagttacgatgggtggacg |
| 274 | | heavy ch aa | EVRLEESGGGLVQPGGSLRLSCAASGFTVSTTYMAWVRQAPGKGLEWVSLIHTGGIFGVGGTS |
| | | | YADSVKGRFTISRDTSKNTVSLQMSSLRVEDTAIYFCAREHRGTIDAFDAWGQGTVVIVSS |
| 275 | | light ch aa | DIHMTQSPSSLSASVGDRVTITCRASQNIRNYLNWYQHKPGKAPKLLIYTTSRLQSGVPSRFS |
| | | | GSGSGTDFTLTVNSLQPEDFASYYCQQSYDGWTFGQGTKVEMK |
| 276 | | heavy ch nuc | gaggtgcgactggaggagtctgggggaggcttggtccagcctggggggtccctgagactctcc |
| | | | tgtgcagcctctggattcaccgtcagtaccacctacatggcctgggtccgccaggctccaggg |
| | | | aaggggctggaatgggtctcacttattcataccggtggcatttttggcgttggcggtacatcc |
| | | | tacgcagactccgtgaagggcagattcaccatctccagagacacttccaagaacacagtgtct |
| | | | cttcaaatgagcagcctgagagtcgaggacacggccatctatttctgtgcgagggaacatcgg |
| | | | ggaactatcgatgcttttgatgcctggggccaagggacagtggtcatcgtctcttcag |
| 277 | | light ch nuc | gacatccacatgacccagtctccatcctccctgtctgcatctgttggagacagagtcaccatc |
| | | | acttgccgggcaagtcagaacattcgaaattatttaaattggtatcaacataaaccagggaaa |
| | | | gcccctaaactcctgatctatactacatcccgtctgcaaagtggggtcccatcaaggttcagt |
| | | | ggcagtggatctgggacagatttcactctcaccgtcaacagcctgcaaccagaagactttgca |
| | | | agttactactgtcaacagagttacgatgggtggacgttcggccaggggaccaaggtggaaatg |
| | | | aaac |
| 278 | 5F1 | CDRH1 aa | GFTFSSYE |
| 279 | | CDRH2 aa | IDFTGSTI |
| 280 | | CDRH3 aa | VRDAGRWGTSWYYFDY |
| 281 | | CDRL1 aa | SSNIGAGYD |
| 282 | | CDRL2 aa | GNN |
| 283 | | CDRL3 aa | QSYDSSLNGWV |
| 284 | | CDRH1 nuc | ggattcactttcagtagctatgag |
| 285 | | CDRH2 nuc | attgattttactggctcaaccatc |
| 286 | | CDRH3 nuc | gtgagagatgcgggccgttggggcaccagttggtactactttgactat |
| 287 | | CDRL1 nuc | agctccaacatcggggcaggttatgat |
| 288 | | CDRL2 nuc | ggtaacaac |
| 289 | | CDRL3 nuc | cagtcgtatgacagcagcctgaatggttgggtg |
| 290 | | heavy ch aa | AVQLVESGGGLAQPGRSLRLSCKVSGFTFSSYEMNWVRQAPGKGLEWIAYIDFTGSTIYYADS |
| | | | VKGRFTISRDTARNSLYLQMNKLRVEDTAVYYCVRDAGRWGTSWYYFDYWGQGTLVTVSS |
| 291 | | light ch aa | QSVLTQPPSVSGAPGQRVTISCTGLSSNIGAGYDIHWYQQIPGKAPKLLIYGNNNRPSGVPDR |
| | | | FSGSKSGTSVSLAITGLQAEDEADYYCQSYDSSLNGWVFGGGTRLTVL |
| 292 | | heavy ch nuc | gcggtgcagctggtggagtctgggggcggcttggcacagcctggacggtccctgaggctctcg |
| | | | tgtaaagtgtctggattcactttcagtagctatgagatgaactgggtccgccaggctccaggg |
| | | | aaggggctggagtggattgcatacattgattttactggctcaaccatctactacgcagactct |
| | | | gtgaagggacgattcaccatttccagagacaccgccaggaactcactctatctgcagatgaac |
| | | | aaattgagagtcgaggacacggctgtttattactgtgtgagagatgcgggccgttggggcacc |
| | | | agttggtactactttgactattggggccagggaaccctggtcaccgtctcctcag |
| 293 | | light ch nuc | cagtctgtgctgacgcagccgccctcagtgtctggggccccagggcagagggtcaccatctcc |
| | | | tgcactgggctcagctccaacatcggggcaggttatgatatacactggtatcagcagattcca |
| | | | ggaaaagcccccaaactcctcatctatggtaacaacaatcggccctcaggggtccctgaccga |
| | | | ttctctggctctaagtctggcacctcagtctccctggccatcactgggctccaggctgaggat |
| | | | gaggctgattattactgccagtcgtatgacagcagcctgaatggttgggtgttcggcggaggg |
| | | | accaggttgaccgtcctaa |
| 294 | | heavy ch aa var 1 | AVQLVESGGDLAQPGRSLRLSCKVSGFTFSSYEMNWVRQAPGKGLEWIAYIDFTGSTIYYADS |
| | | | VKGRFTISRDTARNSLYLQMNKLRVEDTAVYYCVRDAGRWGTSWYYFDYWGQGTLVTVSS |
| 295 | | heavy ch nuc var 1 | gcggtgcagctggtggagtctgggggcgacttggcacagcctggacggtccctgaggctctcg |
| | | | tgtaaagtgtctggattcactttcagtagctatgagatgaactgggtccgccaggctccaggg |
| | | | aaggggctggagtggattgcatacattgattttactggctcaaccatctactacgcagactct |
| | | | gtgaagggacgattcaccatttccagagacaccgccaggaactcactctatctgcagatgaac |
| | | | aaattgagagtcgaggacacggctgtttattactgtgtgagagatgcgggccgttggggcacc |
| | | | agttggtactactttgactattggggccagggaaccctggtcaccgtctcctcag |
| 296 | 4H9.6 | CDRH1 aa | GFTFSSHE |
| 297 | | CDRH2 aa | IDFTGSII |
| 298 | | CDRH3 aa | ARDGGRWGTSWYYFDY |
| 299 | | CDRL1 aa | SSNFGAGYD |
| 300 | | CDRL2 aa | GS |
| 301 | | CDRL3 aa | QSYDSSLSAWV |
| 302 | | CDRH1 nuc | ggattcaccttcagttctcatgag |
| 303 | | CDRH2 nuc | attgattttactggcagtattata |
| 304 | | CDRH3 nuc | gcgagagatgggggtcgttggggcaccagttggtactactttgactac |
| 305 | | CDRL1 nuc | agttccaacttcggggcaggttatgat |
| 306 | | CDRL2 nuc | ggtagc |
| 307 | | CDRL3 nuc | cagtcctatgacagcagcctgagcgcttgggtg |
| 308 | | heavy ch aa | AVQLVESGGGLVRPGGSLRLSCAASGFTFSSHEMHWVRQAPGKGLEWLSYIDFTGSIIYYADS |
| | | | VRGRFTISRDNTKKSLFLQMNSLRDEDTALYYCARDGGRWGTSWYYFDYWGQGVLVTVSS |
| 309 | | light ch aa | QSVLTQPPSVSGAPGQRVTITCTGSSSNFGAGYDGHWYQQLPGTAPKLLIYGSNRPSGVPDRF |
| | | | SGSKSGTSVSLAITGLQADDEADYYCQSYDSSLSAWVFGGGTKLTVL |
| 310 | | heavy ch nuc | gcggtgcagctggtggagtctgggggaggcttggtacggcctggagggtccctgagactctcc |
| | | | tgtgcagcctctggattcaccttcagttctcatgagatgcactgggtccgccaggctccaggg |
| | | | aaggggctggaatggctttcatacattgattttactggcagtattatatactacgcagactct |
| | | | gtgaggggtcggttcaccatctccagagacaacaccaaaaagtcactgtttctgcaaatgaac |
| | | | agcctgagagacgaggatacggctctttattactgtgcgagagatgggggtcgttggggcacc |
| | | | agttggtactactttgactactggggccagggagtcctggtcaccgtctcctcag |
| 311 | | light ch nuc | cagtctgtgctgacgcagccgccctcagtgtctggggccccagggcagagggtcaccataacc |
| | | | tgcactgggagcagttccaacttcggggcaggttatgatggacactggtaccagcaacttcca |
| | | | ggaacagcccccaaactcctcatctatggtagcaatcggccctcaggggtccctgaccgattc |
| | | | tctggctccaagtctggcacctcagtctccctggccatcactgggctccaggctgacgatgag |
| | | | gctgattattactgccagtcctatgacagcagcctgagcgcttgggtgttcggcggagggacc |
| | | | aagctgaccgtcctac |
| 312 | | CDRH2aa var 1 | IDFTGSSI |
| 313 | | CDRH2 nuc var 1 | attgattttactggcagtagtata |
| 314 | | Heavy ch aa var 1 | AVQLVESGGGLVRPGGSLRLSCAASGFTFSSHEMHWVRQAPGKGLEWLSYIDFTGSSIYYADS |
| | | | VRGRFTISRDNTKKSLFLQMNSLRDEDTALYYCARDGGRWGTSWYYFDYWGQGVLVTVSS |
| 315 | | Heavy ch nuc var 1 | gcggtgcagctggtggagtctgggggaggcttggtacggcctggagggtccctgagactctcc |
| | | | tgtgcagcctctggattcaccttcagttctcatgagatgcactgggtccgccaggctccaggg |
| | | | aaggggctggaatggctttcatacattgattttactggcagtagtatatactacgcagactct |
| | | | gtgaggggtcggttcaccatctccagagacaataccaaaaagtcactgtttctgcaaatgaac |
| | | | agcctgagagacgaggatacggctctttattactgtgcgagagatgggggtcgttggggcacc |
| | | | agttggtactactttgactactggggccagggagtcctggtcaccgtctcctcag |
| 316 | 7H3 | CDRH1 aa | GYTFTDYY |
| 317 | | CDRH2 aa | FNPNSGGT |
| 318 | | CDRH3 aa | AKDSAKTASAYYGLNFFYYGMDV |
| 319 | | CDRL1 aa | SSNIGKNY |
| 320 | | CDRL2 aa | KNN |
| 321 | | CDRL3 aa | SAWDGSLSRPL |
| 322 | | CDRH1 nuc | ggatacaccttcaccgactactat |
| 323 | | CDRH2 nuc | ttcaaccctaacagtggtggcaca |
| 324 | | CDRH3 nuc | gcgaaagattccgcgaaaactgcgagtgcttattatggactgaacttcttctactacggtatg |
| | | | gacgtc |
| 325 | | CDRL1 nuc | agttccaacatcggaaagaattat |
| 326 | | CDRL2 nuc | aagaataat |
| 327 | | CDRL3 nuc | tcagcgtgggatggcagcctgagtcgtccacta |
| 328 | | heavy ch aa | QVQLVQSGAEVKNPGASVKVSCKASGYTFTDYYIHWVRQAPGQGLEWMGWFNPNSGGTNFVQN |
| | | | FQGRVTMTRDTSISTAYMELSRLRSDDTAMYYCAKDSAKTASAYYGLNFFYYGMDVWGQGTTV |
| | | | TVSS |
| 329 | | light ch aa | QSVLSQPPSASGTPGQRVTISCSGSSSNIGKNYVYWYQQVPGTAPKLLMFKNNQRPSGVPDRF |
| | | | SGSKSGTSASLAISGLRSEDEADYYCSAWDGSLSRPLFGGGTKVTVL |
| 330 | | heavy ch nuc | caggtgcagctggtgcagtctggggctgaagtgaagaaccctggggcctcagtgaaggtctcc |
| | | | tgcaaggcttctggatacaccttcaccgactactatatacactgggtgcgacaggcccctgga |
| | | | caaggacttgagtggatgggctggttcaaccctaacagtggtggcacaaactttgtacagaac |
| | | | tttcagggcagggtcaccatgaccagggacacgtccatcagcacagcctacatggagctcagc |
| | | | aggctgagatctgacgacacggccatgtattactgtgcgaaagattccgcgaaaactgcgagt |
| | | | gcttattatggactgaacttcttctactacggtatggacgtctggggccaagggaccacggtc |
| | | | accgtctcctcag |
| 331 | | light ch nuc | cagtctgtactgagtcagccaccctcagcatctgggacccccgggcagagggtcaccatctct |
| | | | tgttctggaagcagttccaacatcggaaagaattatgtatattggtaccagcaggtcccagga |
| | | | acggcccccaaactcctcatgtttaagaataatcagcgaccctcaggggtccctgaccgattc |
| | | | tctggctccaagtctggcacctctgcctccctggccatcagtgggctccggtccgaggatgag |
| | | | gctgattattattgttcagcgtgggatggcagcctgagtcgtccactattcggcggagggacc |
| | | | aaggtgaccgtcctag |
| 332 | | CDRH3 aa var 1 | ARDSAKTASAYYGLNFFYYGMDV |
| 333 | | CDRH3 nuc var 1 | gcgagagattccgcgaaaactgcgagtgcttattatggactgaacttcttctactacggtatg |
| | | | gacgtc |
| 334 | | heavy ch aa var 1 | QVQLVQSGAEVKPNPGASVKVSCKASGYTFTDYYIHWVRQAPGQGLEWMGWFNPNSGGTNFVQ |
| | | | NFQGRVTMTRDTSISTAYMELSRLRSDDTAMYYCARDSAKTASAYYGLNFFYYGMDVWGQGTT |
| | | | VTVSS |
| 335 | | heavy ch nuc var 1 | caggtgcagctggtgcagtctggggctgaagtgaagaaccctggggcctcagtgaaggtctcc |
| | | | tgcaaggcttctggatacaccttcaccgactactatatacactgggtgcgacaggcccctgga |
| | | | caaggacttgagtggatgggctggttcaaccctaacagtggtggcacaaactttgtacagaac |
| | | | tttcagggcagggtcaccatgaccagggacacgtccatcagcacagcctacatggagctcagc |
| | | | aggctgagatctgacgacacggccatgtattactgtgcgagagattccgcgaaaactgcgagt |
| | | | gcttattatggactgaacttcttctactacggtatggacgtctggggccaagggaccacggtc |
| | | | accgtctcctcag |
| 336 | 6B4 | CDRH1 aa | GFRFNEFN |
| 337 | | CDRH2 aa | ISIDGRHK |
| 338 | | CDRH3 aa | VTDGKAVDGFSGILEF |
| 339 | | CDRL1 aa | QSVGGY |
| 340 | | CDRL2 aa | DAS |
| 341 | | CDRL3 aa | QQRNNWPPLT |
| 342 | | CDRH1 nuc | ggattcaggttcaatgaatttaat |
| 343 | | CDRH2 nuc | atctcaattgatgggagacacaaa |
| 344 | | CDRH3 nuc | gtgacagatgggaaagcagtggatgggttttccggaattttagagttc |
| 345 | | CDRL1 nuc | cagagtgttggcggctac |
| 346 | | CDRL2 nuc | gatgcatcc |
| 347 | | CDRL3 nuc | cagcagcgtaacaactggccaccactcact |
| 348 | | heavy ch aa | QVQLVESGGGVVQPGRSLRLSCAASGFRFNEFNMHWVRQAPGKGLEWVAVISIDGRHKYNADS |
| | | | VEGRFTISRDNSRNTLYLQMNSLRVEDTALYYCVTDGKAVDGFSGILEFWGQGTPVTVST |
| 349 | | light ch aa | EIVLTQSPATLSLSPGERATLSCWASQSVGGYLAWYQQKPGQAPRLLIYDASIRATGIPARFS |
| | | | GSGSGTHFTLTINSLEPEDFAVYYCQQRNNWPPLTFGGGTKVEIK |
| 350 | | heavy ch nuc | caggtgcaactggtggagtctgggggaggcgtggtccagcctgggaggtccctgagactctcc |
| | | | tgtgcagcctctggattcaggttcaatgaatttaatatgcactgggtccgccaggctccaggc |
| | | | aagggcctggagtgggtggcagttatctcaattgatgggagacacaaatacaacgcagactcc |
| | | | gtggagggccgattcaccatctccagagacaattccagaaacactctttatctgcaaatgaac |
| | | | agcctgagagttgaggacacggctctttattactgtgtgacagatgggaaagcagtggatggg |
| | | | ttttccggaattttagagttctggggccagggaaccccagtcaccgtctccacag |
| 351 | | light ch nuc | gaaattgtgttgacacagtctccggccaccctgtctttgtctccaggggagagagccaccctc |
| | | | tcctgctgggccagtcagagtgttggcggctacttagcctggtaccaacaaaaacctggccag |
| | | | gctcccaggctcctcatctatgatgcatccatcagggccactggcatcccagccaggttcagt |
| | | | ggcagtgggtctgggacacacttcactctcaccatcaatagcctcgagcctgaagattttgcc |
| | | | gtttattactgtcagcagcgtaacaactggccaccactcactttcggcggagggaccaaggtg |
| | | | gagatcaaac |
| 352 | 10C6 | CDRH1 aa | GFSFSNFE |
| 353 | | CDRH1 nuc | ggattcagtttcagtaactttgag |
| 354 | | CDRH2 nuc | attgattttactggctctaccatc |
| 355 | | CDRH3 nuc | gtgagagatgcgggccgttggggcaccagttggtactattttgactat |
| 356 | | CDRL3 nuc | cagtcatatgacagcagcctgaatggttgggtg |
| 357 | | heavy ch aa | AVQLVESGGGLAQPGRSLRLSCKVSGFSFSNFEMNWVRQAPGKGLEWIAYIDFTGSTIYYSDS |
| | | | VKGRFTISRDTARNSLYLQMNKLRVEDTAVYYCVRDAGRWGTSWYYFDYWGQGTLVTVSS |
| 358 | | heavy ch nuc | gcggtgcagctggtggaatccgggggcggcttggcacagcctggacggtccctgaggctctcg |
| | | | tgtaaagtgtccggattcagtttcagtaactttgagatgaactgggtccgccaggctccaggg |
| | | | aaggggctggagtggattgcatatattgattttactggctctaccatctactactcagactct |
| | | | gtgaagggacggtttaccatttccagagacaccgccaggaactcactctatctgcagatgaac |
| | | | aaattgagagtcgaggacacggctgtttattactgtgtgagagatgcgggccgttggggcacc |
| | | | agttggtactattttgactattggggccagggcaccctggtcaccgtctcctcag |
| 359 | | light ch nuc | Cagtctgtgctgacgcagccgccctcagtgtctggggccccagggcagagggtcaccatctcc |
| | | | tgcactgggctcagctccaacatcggggcaggttatgatatacactggtatcagcagattcca |
| | | | ggaaaagcccccaaactcctcatctatggtaacaacaatcggccctcaggggtccctgaccga |
| | | | ttctctggctctaagtctggcacctcagtctccctggccatcactgggctccaggctgaggat |
| | | | gaggctgattattactgccagtcatatgacagcagcctgaatggttgggtgttcggcggaggg |
| | | | accaggttgaccgtcctaa |
| 360 | 2B11 | CDRH1 aa | GFTFGSYE |
| 361 | | CDRL3 aa | QSYDNSLNGWV |
| 362 | | CDRH1 nuc | ggattcaccttcggaagctatgaa |
| 363 | | CDRH2 nuc | attgactttactggttcaaccatc |
| 364 | | CDRH3 nuc | gtgagagatgcgggccgctggggcaccagttggtattactttgactat |
| 365 | | CDRL2 nuc | ggcaacaac |
| 366 | | CDRL3 nuc | cagtcctatgacaacagcctgaatggttgggtg |
| 367 | | heavy ch aa | AVRLVESGGGLAQPGRSLRLSCQVSGFTFGSYEMNWVRQAPGKGLEWIAYIDFTGSTIYYADS |
| | | | VKGRFTISRNTARNSLYLQMNSLRVEDTAVYYCVRDAGRWGTSWYYFDYWGQGTRVTVSP |
| 368 | | light ch aa | QSVLTQPPSVSGAPGQRVTISCTGISSNIGAGYDIHWYQQIPGKAPKLLVYGNNNRPSGVPDR |
| | | | FSGSKSGTSVSLAITGLQVEDEADYYCQSYDNSLNGWVFGGGTRLTVL |
| 369 | | heavy ch nuc | gcggtgcggctggtggagtctgggggaggcttggcacagcctggacggtccctgagactctcg |
| | | | tgtcaagtgtctggattcaccttcggaagctatgaaatgaactgggtccgccaggctcccggc |
| | | | aagggactggagtggattgcctacattgactttactggttcaaccatctactacgcagactct |
| | | | gtgaagggccgattcaccatatccagaaacaccgccaggaactcactctatctgcagatgaac |
| | | | agcctgagagtcgaggacacggctgtttattactgtgtgagagatgcgggccgctggggcacc |
| | | | agttggtattactttgactattggggccaaggaacccgggtcaccgtctccccag |
| 370 | | light ch nuc | cagtctgtgctgacgcagccgccctcagtgtctggggccccagggcagagggtcaccatctcc |
| | | | tgcactgggatcagctccaacatcggggcaggttatgatatacactggtatcagcagattcca |
| | | | ggaaaagcccccaaactcctcgtctatggcaacaacaatcggccctcaggagtccctgaccga |
| | | | ttctctggctctaagtctggcacctcagtctccctggccatcactgggctccaggttgaggat |
| | | | gaggctgattattactgccagtcctatgacaacagcctgaatggttgggtgttcggcggaggg |
| | | | accaggttgaccgtcctaa |

1. A method of neutralizing hCMV infection, comprising the steps of:
(a) administering a dose via injection or infusion of a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively; and
(b) administering a dose via injection or infusion of a second antibody or antigen binding fragment thereof, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively;
wherein the first antibody or antigen binding fragment thereof is administered at a dosage of about 1 to about 50 mg/kg body weight, and the second antibody or antigen binding fragment thereof is administered at a dosage of about 0.1 to about 5.0 mg/kg body weight,
wherein steps (a) and (b) can be performed simultaneously or in any order, and
wherein steps (a) and/or (b) can optionally be repeated to administer multiple doses.

2. The method of claim 1, wherein in (a) the CDRH3 sequence is SEQ ID NO: 332.

3. The method of claim 1, wherein the ratio of the first antibody or fragment to the second antibody or fragment is between about 7.5:1 and about 12.5:1.

4. The method of claim 2, wherein the ratio of the dose of the first antibody or fragment to the second antibody or fragment is about 10:1.

5. The method of claim 1, wherein the first antibody or antigen binding fragment thereof is administered at a dosage of about 2.5 to about 25 mg/kg body weight, and the second antibody or antigen binding fragment thereof is administered at a dosage of about 0.25 to about 2.5 mg/kg body weight.

6. The method of claim 1, wherein the first antibody or antigen binding fragment thereof is administered at a dosage of about 5 to about 10 mg/kg body weight, and the second antibody or antigen binding fragment thereof is administered at a dosage of about 0.5 to about 1 mg/kg body weight.

7. The method of claim 1, wherein the first antibody or antigen binding fragment thereof is administered at a dosage of about 5 mg/kg body weight, and the second antibody or antigen binding fragment thereof is administered at a dosage of about 0.5 mg/kg body weight.

8. The method of claim 1, wherein the first and second antibody or fragment are in lyophilized form.

9. The method of claim 8, wherein the first and second antibody or fragment are reconstituted prior to injection or infusion.

10. The method of claim 9, wherein the first and second antibody or fragment are reconstituted in a pharmaceutical carrier.

11. The method of claim 10, wherein the pharmaceutical carrier is for injection or infusion into an immunocompromised or immunosuppressed subject.

12. The method of claim 10, wherein the pharmaceutical carrier is for injection or infusion into a pregnant subject.

13. The method of claim 1, wherein the doses are administered intraperitoneally, orally, subcutaneously, intramuscularly, topically or intravenously.

14. The method of claim 1, wherein the doses of the first and second antibody or antigen binding fragment thereof are administered on the same day.

15. The method of claim 1, wherein the doses are each administered as a single dosage.

16. The method of claim 1, wherein the doses are each administered as multiple doses.

17. The method of claim 1, wherein the doses are administered about every week, every two weeks, every three weeks, every four weeks, every month, ever month and a half, or every two months.

18. The method of claim 1, wherein the doses are administered over a period of about six months, about 9 months, or about one year.

19. (canceled)

20. (canceled)

21. (canceled)

22. The method of claim 1, wherein the dosage range is a minimum trough serum concentration of at least about 7.4 μg/ml for the first antibody; and a minimum trough serum concentration of at least about 0.74 μg/ml for the second antibody.

23. The method of claim 22, wherein the method decreases the development or risk of development of viral resistance to either antibody or fragment.

24. A composition comprising:
(a) a first antibody or antigen binding fragment thereof, which binds hCMV glycoprotein gB and comprises the CDRH1 sequence of SEQ ID NO: 316, the CDRH2 sequence of SEQ ID NO: 317, and the CDRH3 sequence of SEQ ID NO: 318 or 332; and the CDRL1, CDRL2, and CDRL3 sequences of SEQ ID NOs: 319, 320, and 321, respectively; and
(b) a second antibody or antigen binding fragment thereof 4I22, which binds to a 5-member complex consisting of hCMV glycoproteins gH, gL, UL128, UL130 and UL131A, and comprises the CDRH1, CDRH2, and CDRH3 sequences of SEQ ID NOs: 49, 50, and 51, respectively, and the CDRL1, CDRL2, and CDRL3 sequences of SEQ ID NOs: 52, 53, and 54, respectively;
wherein the ratio of the first antibody or fragment to the second antibody or fragment is between about 7.5:1 and about 12.5:1.

25. The composition of claim 24, wherein in (a) the CDRH3 sequence is SEQ ID NO: 332.

26. The composition of claim 24, wherein the ratio of the dose first antibody or fragment to the second antibody or fragment is about 10:1.

27. The composition of claim 24, wherein the first and second antibody or fragment are in lyophilized form.

28. The composition of claim 27, wherein the first and second antibody or fragment are reconstituted prior to injection or infusion.

29. The composition of claim 28, wherein the first and second antibody or fragment are reconstituted in a pharmaceutical carrier.

30. The composition of claim 29, wherein the pharmaceutical carrier is for injection or infusion into an immunocompromised subject.

31. The composition of claim 29, wherein the pharmaceutical carrier is for injection or infusion into a pregnant subject.

32. (canceled)


SEQUENCE LISTING (370 sequences)

SEQ ID NO: 1 - protein, 8 aa, Artificial Sequence
  source: /note=“Description of Artificial Sequence Synthetic peptide”
GGTFSSYV

SEQ ID NO: 2 - protein, 8 aa, Artificial Sequence
  source: /note=“Description of Artificial Sequence Synthetic peptide”
VIPIFDTV

SEQ ID NO: 3 - protein, 21 aa, Artificial Sequence
  source: /note=“Description of Artificial Sequence Synthetic peptide”
ARGILAYCGGDCYNTPYGMDV

SEQ ID NO: 4 - protein, 6 aa, Artificial Sequence
  source: /note=“Description of Artificial Sequence Synthetic peptide”
QSISSW

SEQ ID NO: 5 - protein, 3 aa, Artificial Sequence
  source: /note=“Description of Artificial Sequence Synthetic peptide”
KAS

SEQ ID NO: 6 - protein, 8 aa, Artificial Sequence
  source: /note=“Description of Artificial Sequence Synthetic peptide”
QQYNSSWT

SEQ ID NO: 7 - DNA, 24 bp, Artificial Sequence
  source: /note=“Description of Artificial Sequence Synthetic oligonucleotide”
ggaggcaccttcagcagctatgtt

SEQ ID NO: 8 - DNA, 24 bp, Artificial Sequence
  source: /note=“Description of Artificial Sequence Synthetic oligonucleotide”
gtcatccctatctttgatacagta

SEQ ID NO: 9 - DNA, 63 bp, Artificial Sequence
  source: /note=“Description of Artificial Sequence Synthetic oligonucleotide”
gcgagaggaattctagcatattgtggtggtgattgctataataccccttacggtatggacgtc

SEQ ID NO: 10 - DNA, 18 bp, Artificial Sequence
  source: /note=“Description of Artificial Sequence Synthetic oligonucleotide”
cagagtattagtagctgg

SEQ ID NO: 11 - DNA, 9 bp, Artificial Sequence
  source: /note=“Description of Artificial Sequence Synthetic oligonucleotide”
aaggcgtct

SEQ ID NO: 12 - DNA, 24 bp, Artificial Sequence
  source: /note=“Description of Artificial Sequence Synthetic oligonucleotide”
caacagtataatagttcgtggacg

SEQ ID NO: 13 - protein, 128 aa, Artificial Sequence
  source: /note=“Description of Artificial Sequence Synthetic polypeptide”
QVQLVQSGAEVKKPGSSVRVSCKASGGTFSSYVIIWVRQAPGQGLEWMGGVIPIFDTVNYAQKFQGRVTITADESTSTAYMELSSLKSEDTAVYYCARGILAYCGGDCYNTPYGMDVWGQGTTVTVSS

SEQ ID NO: 14 - protein, 106 aa, Artificial Sequence
  source: /note=“Description of Artificial Sequence Synthetic polypeptide”
DIQMTQSPSILSASVGDRVTITCRASQSISSWLAWYQQKPGKAPKLLIYKASSLEIGVPSRISGSGSGTEFTLTISSLQPDDFATYYCQQYNSSWTFGQGTKVEIK

SEQ ID NO: 15 - DNA, 385 bp, Artificial Sequence
  source: /note=“Description of Artificial Sequence Synthetic polynucleotide”
caggtgcagctggtgcagtctggggctgaggtgaagaagcctgggtcctcggtgagggtctcctgcaaggcttctggaggcaccttcagcagctatgttatcatctgggtgcgacaggcccctggacaaggtcttgagtggatggggggggtcatccctatctttgatacagtaaattacgcacagaagttccagggcagagtcacgattaccgcggacgaatccacgagtactgcctacatggagctgagcagcctgaaatctgaggacacggccgtatattactgtgcgagaggaattctagcatattgtggtggtgattgctataataccccttacggtatggacgtctggggccaagggaccacggtcaccgtctcctcag

SEQ ID NO: 16 - DNA, 319 bp, Artificial Sequence
  source: /note=“Description of Artificial Sequence Synthetic polynucleotide”
gacatccagatgacccagtctccttccatcctgtctgcatctgtaggagacagagtcaccatcacttgccgggccagtcagagtattagtagctggttggcctggtatcagcagaaaccagggaaagccccaaaactcctaatctataaggcgtctagtttagaaattggggtcccatcaaggatcagcggcagtggatctgggacagaattcactctcaccatcagcagcctgcagcctgatgattttgcaacttattactgccaacagtataatagttcgtggacgttcggccaagggacgaaggtggaaatcaaac

SEQ ID NO: 17 - protein, 8 aa, Artificial Sequence
  source: /note=“Description of Artificial Sequence Synthetic peptide”
GFTFGDYA

SEQ ID NO: 18 - protein, 10 aa, Artificial Sequence
  source: /note=“Description of Artificial Sequence Synthetic peptide”
IRSKAYGGTT

SEQ ID NO: 19 - protein, 18 aa, Artificial Sequence
  source: /note=“Description of Artificial Sequence Synthetic peptide”
TRASSLLWLLNPQPNFDY

SEQ ID NO: 20 - protein, 6 aa, Artificial Sequence
  source: /note=“Description of Artificial Sequence Synthetic peptide”
NIGSNN

SEQ ID NO: 21 - protein, 3 aa, Artificial Sequence
  source: /note=“Description of Artificial Sequence Synthetic peptide”
DDS

SEQ ID NO: 22 - protein, 11 aa, Artificial Sequence
  source: /note=“Description of Artificial Sequence Synthetic peptide”
QVWDSSSDHPV

SEQ ID NO: 23 - DNA, 24 bp, Artificial Sequence
  source: /note=“Description of Artificial Sequence Synthetic oligonucleotide”
ggattcacctttggtgattatgct

SEQ ID NO: 24 - DNA, 30 bp, Artificial Sequence
  source: /note=“Description of Artificial Sequence Synthetic oligonucleotide”
attagaagcaaagcttatggtgggacaaca

SEQ ID NO: 25 - DNA, 54 bp, Artificial Sequence
  source: /note=“Description of Artificial Sequence Synthetic oligonucleotide”
actagagcatcttcattactatggttactaaaccctcaacccaactttgactac

SEQ ID NO: 26 - DNA, 18 bp, Artificial Sequence
  source: /note=“Description of Artificial Sequence Synthetic oligonucleotide”
aacattggaagtaacaat

SEQ ID NO: 27 - DNA, 9 bp, Artificial Sequence
  source: /note=“Description of Artificial Sequence Synthetic oligonucleotide”
gatgatagc

SEQ ID NO: 28 - DNA, 33 bp, Artificial Sequence
  source: /note=“Description of Artificial Sequence Synthetic oligonucleotide”
caggtgtgggatagtagtagtgatcatccggta

SEQ ID NO: 29 - protein, 127 aa, Artificial Sequence
  source: /note=“Description of Artificial Sequence Synthetic polypeptide”
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTRASSLLWLLNPQPNFDYWGQGTLVTVSS

SEQ ID NO: 30 - protein, 108 aa, Artificial Sequence
  source: /note=“Description of Artificial Sequence Synthetic polypeptide”
SYVLTQPPSVSVAPGQTARITCGGNNIGSNNVHWYQQKPGQAPVLVVYDDSDRPSGIPERFSGSNSGNTATLTISRVEAGDEADYYCQVWDSSSDHPVFGGGTKLTVL

SEQ ID NO: 31 - DNA, 382 bp, Artificial Sequence
  source: /note=“Description of Artificial Sequence Synthetic polynucleotide”
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagagcatcttcattactatggttactaaaccctcaacccaactttgactactggggccagggaaccctggtcaccgtctcctcag

SEQ ID NO: 32 - DNA, 325 bp, Artificial Sequence
  source: /note=“Description of Artificial Sequence Synthetic polynucleotide”
tcctatgtgctgactcagccaccctcggtgtcagtggccccaggacagacggccaggattacctgtgggggaaacaacattggaagtaacaatgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccggtattcggcggagggaccaagctgaccgtcctag

SEQ ID NO: 33 - protein, 8 aa, Artificial Sequence
  source: /note=“Description of Artificial Sequence Synthetic peptide”
GFTFHNYR

SEQ ID NO: 34 - protein, 8 aa, Artificial Sequence
  source: /note=“Description of Artificial Sequence Synthetic peptide”
IKQDGSEK

SEQ ID NO: 35 - protein, 19 aa, Artificial Sequence
  source: /note=“Description of Artificial Sequence Synthetic peptide”
ARGEGYTYGVVYSYSAMDV

SEQ ID NO: 36 - protein, 6 aa, Artificial Sequence
  source: /note=“Description of Artificial Sequence Synthetic peptide”
VLPNQY

SEQ ID NO: 37 - protein, 3 aa, Artificial Sequence
  source: /note=“Description of Artificial Sequence Synthetic peptide”
KDT

SEQ ID NO: 38 - protein, 11 aa, Artificial Sequence
  source: /note=“Description of Artificial Sequence Synthetic peptide”
QSADSSGADYV

SEQ ID NO: 39 - DNA, 24 bp, Artificial Sequence
  source: /note=“Description of Artificial Sequence Synthetic oligonucleotide”
ggattcacctttcataactatcgc

SEQ ID NO: 40 - DNA, 24 bp, Artificial Sequence
  source: /note=“Description of Artificial Sequence Synthetic oligonucleotide”
ataaagcaagatggaagtgagaaa

SEQ ID NO: 41 - DNA, 57 bp, Artificial Sequence
  source: /note=“Description of Artificial Sequence Synthetic oligonucleotide”
gcgaggggtgaagggtacacctatggtgtcgtctactcctattccgctatggacgtc

SEQ ID NO: 42 - DNA, 18 bp, Artificial Sequence
  source: /note=“Description of Artificial Sequence Synthetic oligonucleotide”
gtattgccaaaccaatat

SEQ ID NO: 43 - DNA, 9 bp, Artificial Sequence
  source: /note=“Description of Artificial Sequence Synthetic oligonucleotide”
aaagacact

SEQ ID NO: 44 - DNA, 33 bp, Artificial Sequence
  source: /note=“Description of Artificial Sequence Synthetic oligonucleotide”
caatcagcagacagcagtggtgccgattatgtc

SEQ ID NO: 45 - protein, 126 aa, Artificial Sequence
  source: /note=“Description of Artificial Sequence Synthetic polypeptide”
EVQLVESGGGLVRPGGSLRLSCAASGFTFHNYRMNWVRQAPGKGLEWVANIKQDGSEKSYVDSVRGRFTTSRDNSKNSLYLQINSLRAEDTAVYYCARGEGYTYGVVYSYSAMDVWGQGTTVIVSS

SEQ ID NO: 46 - protein, 108 aa, Artificial Sequence
  source: /note=“Description of Artificial Sequence Synthetic polypeptide”
SYELTQPPSVSVSPGQTARITCSGNVLPNQYASWYQQKPGQAPVLVIYKDTERPSGIPGRFSGSSSGTTVTLTISGVQAEDEADYYCQSADSSGADYVFGTGTKVTVL

SEQ ID NO: 47 - DNA, 379 bp, Artificial Sequence
  source: /note=“Description of Artificial Sequence Synthetic polynucleotide”
gaggtgcagctggtagagtctgggggaggcttggtccggcctggggggtccctgagactctcatgtgcagcctctggattcacctttcataactatcgcatgaactgggtccgccaggctccagggaaggggctggagtgggtggccaacataaagcaagatggaagtgagaaatcctatgtggactctgtgaggggccgattcaccacctccagagacaactccaagaattcactctatctgcaaattaacagcctgcgagccgaggacacggctgtctattactgtgcgaggggtgaagggtacacctatggtgtcgtctactcctattccgctatggacgtctggggccaagggaccacagtcatcgtctcctcag

SEQ ID NO: 48 - DNA, 325 bp, Artificial Sequence
  source: /note=“Description of Artificial Sequence Synthetic polynucleotide”
tcctatgagctgacacagccaccctcggtgtcagtgtccccaggacagacggccaggatcacctgctctggaaatgtattgccaaaccaatatgcttcttggtaccagcagaagccaggccaggcccctgtattggtgatatataaagacactgagaggccctcagggatccctgggcgattctctggctccagctcagggacgacagtcacgttgaccatcagtggagtccaggcagaggacgaggctgactattactgtcaatcagcagacagcagtggtgccgattatgtcttcggaactgggaccaaggtcaccgtcctag

SEQ ID NO: 49 - protein, 8 aa, Artificial Sequence
  source: /note=“Description of Artificial Sequence Synthetic peptide”
GFTFSSYA

SEQ ID NO: 50 - protein, 8 aa, Artificial Sequence
  source: /note=“Description of Artificial Sequence Synthetic peptide”
ISYDGDNK

SEQ ID NO: 51 - protein, 19 aa, Artificial Sequence
  source: /note=“Description of Artificial Sequence Synthetic peptide”
AREELVGLMPPYYNYGLDV

SEQ ID NO: 52 - protein, 8 aa, Artificial Sequence
  source: /note=“Description of Artificial Sequence Synthetic peptide”
NSNIGNNY

SEQ ID NO: 53 - protein, 3 aa, Artificial Sequence
  source: /note=“Description of Artificial Sequence Synthetic peptide”
DND

SEQ ID NO: 54 - protein, 12 aa, Artificial Sequence
  source: /note=“Description of Artificial Sequence Synthetic peptide”
ETWDTSLSAAVV

SEQ ID NO: 55 - DNA, 24 bp, Artificial Sequence
  source: /note=“Description of Artificial Sequence Synthetic oligonucleotide”
ggattcaccttcagttcctatgct

SEQ ID NO: 56 - DNA, 24 bp, Artificial Sequence
  source: /note=“Description of Artificial Sequence Synthetic oligonucleotide”
atttcatatgatggcgacaacaaa

SEQ ID NO: 57 - DNA, 57 bp, Artificial Sequence
  source: /note=“Description of Artificial Sequence Synthetic oligonucleotide”
gcgagagaagagttagtcgggttgatgcctccctattacaactacggattggacgtc

SEQ ID NO: 58 - DNA, 24 bp, Artificial Sequence
  source: /note=“Description of Artificial Sequence Synthetic oligonucleotide”
aactccaacatcgggaataattat

SEQ ID NO: 59 - DNA, 9 bp, Artificial Sequence
  source: /note=“Description of Artificial Sequence Synthetic oligonucleotide”
gacaatgat

SEQ ID NO: 60 - DNA, 36 bp, Artificial Sequence
  source: /note=“Description of Artificial Sequence Synthetic oligonucleotide”
gaaacatgggataccagcctgagtgctgctgttgtc

SEQ ID NO: 61 - protein, 126 aa, Artificial Sequence
  source: /note=“Description of Artificial Sequence Synthetic polypeptide”
QVQLVESGGGVVQPGRSLRLSCVASGFTFSSYAMHWVRQAPGKGLEWVAVISYDGDNKFYADSVKGRFRISRDTSKNTLYLEMNSLRAADTAIYYCAREELVGLMPPYYNYGLDVWGQGTTVTVSS

SEQ ID NO: 62 - protein, 111 aa, Artificial Sequence
  source: /note=“Description of Artificial Sequence Synthetic polypeptide”
QSVLTQPPSVSAAPGQKVTISCSGSNSNIGNNYVSWYQQLPGRAPKLLIYDNDHRPSGIPDRFSGSKSGTSATLVITGLQTGDEADYYCETWDTSLSAAVVFGGGTKLTVL

SEQ ID NO: 63 - DNA, 379 bp, Artificial Sequence
  source: /note=“Description of Artificial Sequence Synthetic polynucleotide”
caggtgcagctggtggagtctgggggaggggtggtccagcctgggaggtccctgagactctcctgtgtagcctctggattcaccttcagttcctatgctatgcactgggtccgccaggctccaggcaagggactggagtgggtggcagttatttcatatgatggcgacaacaaattctacgcagactccgtgaagggccgattcaggatctccagagacacatccaagaatacactgtatctggaaatgaacagcctgagagctgcggacacggctatatattactgtgcgagagaagagttagtcgggttgatgcctccctattacaactacggattggacgtctggggccaaggaaccacggtcaccgtctcgtcag

SEQ ID NO: 64 - DNA, 334 bp, Artificial Sequence
  source: /note=“Description of Artificial Sequence Synthetic polynucleotide”
cagtctgtgttgactcagccgccctcagtgtctgcggccccaggacagaaggtcaccatctcctgctctggaagcaactccaacatcgggaataattatgtatcgtggtaccagcagctcccaggaagagcccccaaactcctcatttatgacaatgatcaccgaccctcagggattcctgaccgattctctggctccaagtctggcacgtcagccaccctggtcatcaccggactccagactggggacgaggccgattattactgcgaaacatgggataccagcctgagtgctgctgttgtcttcggcggagggaccaagctgaccgtcctac

SEQ ID NO: 65 - protein, 10 aa, Artificial Sequence
  source: /note=“Description of Artificial Sequence Synthetic peptide”
GFSLNTNGVG

SEQ ID NO: 66 - protein, 7 aa, Artificial Sequence
  source: /note=“Description of Artificial Sequence Synthetic peptide”
IYWNGNE

SEQ ID NO: 67 - protein, 17 aa, Artificial Sequence
  source: /note=“Description of Artificial Sequence Synthetic peptide”
VHWPQGLTTVTRLAFDI

SEQ ID NO: 68 - protein, 9 aa, Artificial Sequence
  source: /note=“Description of Artificial Sequence Synthetic peptide”
TSDVGRYNF

SEQ ID NO: 69 - protein, 3 aa, Artificial Sequence
  source: /note=“Description of Artificial Sequence Synthetic peptide”
DVS

SEQ ID NO: 70 - protein, 12 aa, Artificial Sequence
  source: /note=“Description of Artificial Sequence Synthetic peptide”
CSYAGGNFFSYV

SEQ ID NO: 71 - DNA, 30 bp, Artificial Sequence
  source: /note=“Description of Artificial Sequence Synthetic oligonucleotide”
ggcttctcactcaacactaatggagtgggt

SEQ ID NO: 72 - DNA, 21 bp, Artificial Sequence
  source: /note=“Description of Artificial Sequence Synthetic oligonucleotide”
atttactggaatggtaatgag

SEQ ID NO: 73 - DNA, 51 bp, Artificial Sequence
  source: /note=“Description of Artificial Sequence Synthetic oligonucleotide”
gtacactggccccaagggttgactacggtgacaagacttgcttttgatatc

SEQ ID NO: 74 - DNA, 27 bp, Artificial Sequence
  source: /note=“Description of Artificial Sequence Synthetic oligonucleotide”
accagtgatgttggtcgttataacttt

SEQ ID NO: 75 - DNA, 9 bp, Artificial Sequence
  source: /note=“Description of Artificial Sequence Synthetic oligonucleotide”
gatgtcagt

SEQ ID NO: 76 - DNA, 36 bp, Artificial Sequence
  source: /note=“Description of Artificial Sequence Synthetic oligonucleotide”
tgctcatatgcaggcggcaattttttctcttatgtc

SEQ ID NO: 77 - protein, 125 aa, Artificial Sequence
  source: /note=“Description of Artificial Sequence Synthetic polypeptide”
QITLRESGPTLVKPTQTLTLTCTFSGFSLNTNGVGVGWIRQPPGKALEWLALIYWNGNEGYSPSLKSRLTITKDTSKNQVVLTMTNMDPVDTATYYCVHWPQGLTTVTRLAFDIWGQGTMVTVSS

SEQ ID NO: 78 - protein, 112 aa, Artificial Sequence
  source: /note=“Description of Artificial Sequence Synthetic polypeptide”
QSALTQPRSVSGSPGQSVTISCTGTTSDVGRYNFVSWYQQHPGKAPKLLMYDVSQRPSGVPSRFSGSKSGNTASLTISGLQAEDEAVFYCCSYAGGNFFSYVFGTGTKVTVL

SEQ ID NO: 79 - DNA, 376 bp, Artificial Sequence
  source: /note=“Description of Artificial Sequence Synthetic polynucleotide”
cagatcaccttgagggagtctggtcctacgctggtgaaacccacacagaccctcacgctgacctgcaccttctctggcttctcactcaacactaatggagtgggtgtgggctggatccgtcagcccccaggaaaggccctggagtggcttgcactcatttactggaatggtaatgagggctacagcccctctctgaaaagcagactcaccatcaccaaggacacctccaaaaaccaggtggtcctgacaatgaccaacatggaccctgtggacacagccacatattactgtgtacactggccccaagggttgactacggtgacaagacttgcttttgatatctggggccaagggactatggtcaccgtctcttcag

SEQ ID NO: 80 - DNA, 337 bp, Artificial Sequence
  source: /note=“Description of Artificial Sequence Synthetic polynucleotide”
cagtctgccctgactcagcctcgctcagtgtccgggtctcctggacagtcagtcaccatctcctgcactggaaccaccagtgatgttggtcgttataactttgtctcctggtaccaacaacacccaggcaaagcccccaaactcctgatgtatgatgtcagtcagcggccctcaggggtccctagtcgcttctctggctccaagtctggcaacacggcctccctgaccatctctgggctccaggctgaggatgaggctgttttttactgctgctcatatgcaggcggcaattttttctcttatgtcttcggaactgggaccaaggtcaccgtcctag

SEQ ID NO: 81 - protein, 8 aa, Artificial Sequence
  source: /note=“Description of Artificial Sequence Synthetic peptide”
GGSIRSYY

SEQ ID NO: 82 - protein, 7 aa, Artificial Sequence
  source: /note=“Description of Artificial Sequence Synthetic peptide”
IYYSGNT

SEQ ID NO: 83 - protein, 14 aa, Artificial Sequence
  source: /note=“Description of Artificial Sequence Synthetic peptide”
ARHDVIVVRGVFDV

SEQ ID NO: 84 - protein, 9 aa, Artificial Sequence
  source: /note=“Description of Artificial Sequence Synthetic peptide”
SSDIGTYNL

SEQ ID NO: 85 - protein, 3 aa, Artificial Sequence
  source: /note=“Description of Artificial Sequence Synthetic peptide”
DGS

SEQ ID NO: 86 - protein, 12 aa, Artificial Sequence
  source: /note=“Description of Artificial Sequence Synthetic peptide”
CSYAGTSDFFVV

SEQ ID NO: 87 - DNA, 24 bp, Artificial Sequence
  source: /note=“Description of Artificial Sequence Synthetic oligonucleotide”
ggtggctccatccggagttactac

SEQ ID NO: 88 - DNA, 21 bp, Artificial Sequence
  source: /note=“Description of Artificial Sequence Synthetic oligonucleotide”
atctattacagtgggaacacc

SEQ ID NO: 89 - DNA, 42 bp, Artificial Sequence
  source: /note=“Description of Artificial Sequence Synthetic oligonucleotide”
gcgagacatgatgtgatagtagtccgcggtgtctttgatgtc

SEQ ID NO: 90 - DNA, 27 bp, Artificial Sequence
  source: /note=“Description of Artificial Sequence Synthetic oligonucleotide”
agcagtgatattggaacttataacctt

SEQ ID NO: 91 - DNA, 9 bp, Artificial Sequence
  source: /note=“Description of Artificial Sequence Synthetic oligonucleotide”
gatggcagt

SEQ ID NO: 92 - DNA, 36 bp, Artificial Sequence
  source: /note=“Description of Artificial Sequence Synthetic oligonucleotide”
tgctcatatgctggtactagcgatttctttgtggtt

SEQ ID NO: 93 - protein, 120 aa, Artificial Sequence
  source: /note=“Description of Artificial Sequence Synthetic polypeptide”
QVQLQESGPGLVKPSETLSLTCTVSGGSIRSYYWSWIRQPPGKGLEWIGHIYYSGNTNYSPSLQSRVTISLDTPKNQFSLRLSSVTAADTAVYYCARHDVIVVRGVFDVWGQGTVVTVSS

SEQ ID NO: 94 - protein, 112 aa, Artificial Sequence
  source: /note=“Description of Artificial Sequence Synthetic polypeptide”
QSALTQPASVSGSPGQSITISCTGTSSDIGTYNLVSWYQQHPGKAPKVLIYDGSKRPSGVSSRFSASKSGNTASLTISGLQAEDETDYYCCSYAGTSDFFVVFGGGTKLTVL

SEQ ID NO: 95 - DNA, 361 bp, Artificial Sequence
  source: /note=“Description of Artificial Sequence Synthetic polynucleotide”
caggtgcagctgcaggagtcgggcccaggtctggtgaagccttcggagaccctgtccctcacctgcactgtctctggtggctccatccggagttactactggagctggatccggcagcccccagggaagggactggagtggattgggcacatctattacagtgggaacaccaactacagcccctccctccagagtcgagtcaccatatcattagacacgcccaagaaccaattctccctgcggctgagctctgtgaccgccgcagacacggccgtctattactgtgcgagacatgatgtgatagtagtccgcggtgtctttgatgtctggggccaagggacagtggtcaccgtctcttcag

SEQ ID NO: 96 - DNA, 337 bp, Artificial Sequence
  source: /note=“Description of Artificial Sequence Synthetic polynucleotide”
cagtctgccctgactcagcctgcctccgtgtctgggtcacctggacagtcgatcaccatctcctgcactggaaccagcagtgatattggaacttataaccttgtctcctggtaccaacaacacccaggcaaagcccccaaagtcctaatttatgatggcagtaagcggccctcaggggtttctagtcgcttctctgcctccaagtctggcaacacggcctccctgacaatctctgggctccaggctgaggacgagactgattattactgctgctcatatgctggtactagcgatttctttgtggttttcggcggagggaccaagctgaccgtcctgg

SEQ ID NO: 97 - protein, 8 aa, Artificial Sequence
  source: /note=“Description of Artificial Sequence Synthetic peptide”
GDTFPAYW

SEQ ID NO: 98 - protein, 8 aa, Artificial Sequence
  source: /note=“Description of Artificial Sequence Synthetic peptide”
IYPIDSET

SEQ ID NO: 99 - protein, 14 aa, Artificial Sequence
  source: /note=“Description of Artificial Sequence Synthetic peptide”
ARGTSTGLREAFHI

SEQ ID NO: 100 - protein, 11 aa, Artificial Sequence
  source: /note=“Description of Artificial Sequence Synthetic peptide”
QSLGYSDGNTY

SEQ ID NO: 101 - protein, 3 aa, Artificial Sequence
  source: /note=“Description of Artificial Sequence Synthetic peptide”
EVS

SEQ ID NO: 102 - protein, 11 aa, Artificial Sequence
  source: /note=“Description of Artificial Sequence Synthetic peptide”
MQGTHWPPMCS

SEQ ID NO: 103 - DNA, 24 bp, Artificial Sequence
  source: /note=“Description of Artificial Sequence Synthetic oligonucleotide”
ggagacacttttcccgcctactgg

SEQ ID NO: 104 - DNA, 24 bp, Artificial Sequence
  source: /note=“Description of Artificial Sequence Synthetic oligonucleotide”
atctatcctattgactctgagacc

SEQ ID NO: 105 - DNA, 42 bp, Artificial Sequence
  source: /note=“Description of Artificial Sequence Synthetic oligonucleotide”
gcccgggggacaagtactggcctcagagaggcttttcatatc

SEQ ID NO: 106 - DNA, 33 bp, Artificial Sequence
  source: /note=“Description of Artificial Sequence Synthetic oligonucleotide”
caaagcctcggatacagtgatggaaacacctat

SEQ ID NO: 107 - DNA, 9 bp, Artificial Sequence
  source: /note=“Description of Artificial Sequence Synthetic oligonucleotide”
gaggtttct

SEQ ID NO: 108 - DNA, 33 bp, Artificial Sequence
  source: /note=“Description of Artificial Sequence Synthetic oligonucleotide”
atgcaaggtacacactggcctcccatgtgcagt

SEQ ID NO: 109 - protein, 121 aa, Artificial Sequence
  source: /note=“Description of Artificial Sequence Synthetic polypeptide”
EVQLVQSGAEVKKPGESLKISCRESGDTFPAYWIAWVRQMPGKGLEWMGIIYPIDSETTYSPSFQGQVTISADKSINTAYLQWSSLKASDSAIYYCARGTSTGLREAFHIWGQGTMVTVSS

SEQ ID NO: 110 - protein, 114 aa, Artificial Sequence
  source: /note=“Description of Artificial Sequence Synthetic polypeptide”
DVVMTQSPLSLAVTLGQPAYISCRSSQSLGYSDGNTYLNWFQQRPGQSPRRLIYEVSNRDSGVPDRFSGSGSGTDFTLKISRVEAEDVGTYYCMQGTHWPPMCSFGQGTKLEIK

SEQ ID NO: 111 - DNA, 364 bp, Artificial Sequence
  source: /note=“Description of Artificial Sequence Synthetic polynucleotide”
gaggtgcagctggtgcagtctggagcagaggtgaaaaagcccggggagtctctgaagatctcctgtagggaatctggagacacttttcccgcctactggatcgcctgggtgcgccagatgcccgggaaaggcctggagtggatgggaattatctatcctattgactctgagaccacatatagcccgtccttccaaggccaggtcaccatttcagccgacaagtccatcaacaccgcctacctgcagtggagcagcctgaaggcctcggactccgccatttattactgtgcccgggggacaagtactggcctcagagaggcttttcatatctggggccaagggacaatggtcaccgtctcttcag

SEQ ID NO: 112 - DNA, 343 bp, Artificial Sequence
  source: /note=“Description of Artificial Sequence Synthetic polynucleotide”
gatgttgtgatgactcagtctccactctccctggccgtcacccttggacagccggcctacatctcctgcaggtcaagtcaaagcctcggatacagtgatggaaacacctatttgaattggtttcagcagagaccaggccaatctcccaggcgcctaatttatgaggtttctaaccgggactctggggtcccagacagattcagcggcagtgggtcgggcactgatttcacactgaaaatcagcagggtggaggctgaggatgttgggacttattactgcatgcaaggtacacactggcctcccatgtgcagttttggccaggggaccaagttggagatcaaac

SEQ ID NO: 113 - protein, 8 aa, Artificial Sequence
  source: /note=“Description of Artificial Sequence Synthetic peptide”
GFTFSNYG

SEQ ID NO: 114 - protein, 8 aa, Artificial Sequence
  source: /note=“Description of Artificial Sequence Synthetic peptide”
IWNDGSKK

SEQ ID NO: 115 - protein, 19 aa, Artificial Sequence
  source: /note=“Description of Artificial Sequence Synthetic peptide”
ARDEGVQMVFAMPDYGMDV

SEQ ID NO: 116 - protein, 6 aa, Artificial Sequence
  source: /note=“Description of Artificial Sequence Synthetic peptide”
KLGDKF

SEQ ID NO: 117 - protein, 3 aa, Artificial Sequence
  source: /note=“Description of Artificial Sequence Synthetic peptide”
QDS

SEQ ID NO: 118 - protein, 11 aa, Artificial Sequence
  source: /note=“Description of Artificial Sequence Synthetic peptide”
QAWDSSTAHYV

SEQ ID NO: 119 - DNA, 24 bp, Artificial Sequence
  source: /note=“Description of Artificial Sequence Synthetic oligonucleotide”
ggattcaccttcagtaattatggc

SEQ ID NO: 120 - DNA, 24 bp, Artificial Sequence
  source: /note=“Description of Artificial Sequence Synthetic oligonucleotide”
atatggaatgatggaagtaagaaa

SEQ ID NO: 121 - DNA, 57 bp, Artificial Sequence
  source: /note=“Description of Artificial Sequence Synthetic oligonucleotide”
gcgagagatgaaggtgtacaaatggtgttcgccatgcctgactacggtatggacgtc

SEQ ID NO: 122 - DNA, 18 bp, Artificial Sequence
  source: /note=“Description of Artificial Sequence Synthetic oligonucleotide”
aaattgggggataaattc

SEQ ID NO: 123 - DNA, 9 bp, Artificial Sequence
  source: /note=“Description of Artificial Sequence Synthetic oligonucleotide”
caagattcc

SEQ ID NO: 124 - DNA, 33 bp, Artificial Sequence
  source: /note=“Description of Artificial Sequence Synthetic oligonucleotide”
caggcgtgggacagcagcactgcccattatgtc

SEQ ID NO: 125 - protein, 126 aa, Artificial Sequence
  source: /note=“Description of Artificial Sequence Synthetic polypeptide”
QVQLLESGGGVVQPGRSLRLSCAASGFTFSNYGMHWVRQAPGKGLEWVAVIWNDGSKKYYAESVKGRFTISRDNSKNTVYLQMNSLRAEDTAVYYCARDEGVQMVFAMPDYGMDVWGQGTTVTVSS

SEQ ID NO: 126 - protein, 108 aa, Artificial Sequence
  source: /note=“Description of Artificial Sequence Synthetic polypeptide”
SYELTQPPSVSVSPGQTASITCSGDKLGDKFACWYQQRPGQSPILVIYQDSKRPSGIPERFSGSNSGNTATLTIRGTQAMDEADYYCQAWDSSTAHYVFGTGTKVTVL

SEQ ID NO: 127 - DNA, 379 bp, Artificial Sequence
  source: /note=“Description of Artificial Sequence Synthetic polynucleotide”
caggtgcagttgctggagtctgggggaggcgtggtccagcctgggaggtccctgagactctcctgtgcagcgtctggattcaccttcagtaattatggcatgcactgggtccgccaggctccaggcaaggggctggagtgggtggcagttatatggaatgatggaagtaagaaatattatgcagagtccgtgaagggccggttcaccatctccagagacaattccaagaacacagtatatctacaaatgaacagcctgagagccgaggacacggctgtgtattactgtgcgagagatgaaggtgtacaaatggtgttcgccatgcctgactacggtatggacgtctggggccaggggaccacggtcaccgtctcctcag

SEQ ID NO: 128 - DNA, 325 bp, Artificial Sequence
  source: /note=“Description of Artificial Sequence Synthetic polynucleotide”
tcctatgaactgactcagccaccctcagtgtccgtgtccccaggacagacagccagcatcacttgctctggagataaattgggggataaattcgcttgctggtatcagcagaggccaggccagtctcctatactggtcatctatcaagattccaagcggccctcagggatccctgagcgattctctggctccaactctgggaacacagccactctgaccatccgcgggacccaggctatggatgaggctgactattactgtcaggcgtgggacagcagcactgcccattatgtcttcggaactgggaccaaggtcaccgtccttg

SEQ ID NO: 129 - protein, 8 aa, Artificial Sequence
  source: /note=“Description of Artificial Sequence Synthetic peptide”
GFSFSNYG

SEQ ID NO: 130 - protein, 8 aa, Artificial Sequence
  source: /note=“Description of Artificial Sequence Synthetic peptide”
IPSDGNYQ

SEQ ID NO: 131 - protein, 10 aa, Artificial Sequence
  source: /note=“Description of Artificial Sequence Synthetic peptide”
AHLGGGLFDF

SEQ ID NO: 132 - protein, 9 aa, Artificial Sequence
  source: /note=“Description of Artificial Sequence Synthetic peptide”
SSDVGGYEF

SEQ ID NO: 133 - protein, 3 aa, Artificial Sequence
  source: /note=“Description of Artificial Sequence Synthetic peptide”
DVD

SEQ ID NO: 134 - protein, 8 aa, Artificial Sequence
  source: /note=“Description of Artificial Sequence Synthetic peptide”
YSSADTWV

SEQ ID NO: 135 - DNA, 24 bp, Artificial Sequence
  source: /note=“Description of Artificial Sequence Synthetic oligonucleotide”
ggattctccttcagtaattatggc

SEQ ID NO: 136 - DNA, 24 bp, Artificial Sequence
  source: /note=“Description of Artificial Sequence Synthetic oligonucleotide”
ataccgtctgatggaaattatcaa

SEQ ID NO: 137 - DNA, 30 bp, Artificial Sequence
  source: /note=“Description of Artificial Sequence Synthetic oligonucleotide”
gcccacctcggggggggtttatttgacttc

SEQ ID NO: 138 - DNA, 27 bp, Artificial Sequence
  source: /note=“Description of Artificial Sequence Synthetic oligonucleotide”
agcagtgatgttggtggttatgagttt

SEQ ID NO: 139 - DNA, 9 bp, Artificial Sequence
  source: /note=“Description of Artificial Sequence Synthetic oligonucleotide”
gatgtcgat

SEQ ID NO: 140 - DNA, 24 bp, Artificial Sequence
  source: /note=“Description of Artificial Sequence Synthetic oligonucleotide”
tactcatctgcagacacctgggtc

SEQ ID NO: 141 - protein, 117 aa, Artificial Sequence
  source: /note=“Description of Artificial Sequence Synthetic polypeptide”
QVQLVESGGGVVQPGGSLRLSCAASGFSFSNYGMHWVRQAPGKGLEWVALIPSDGNYQYYTDSVKGRFTVSRDNSRNTLYLQMKSLRAEDTARYHCAHLGGGLFDFWGQGTLVTVSS

SEQ ID NO: 142 - protein, 108 aa, Artificial Sequence
  source: /note=“Description of Artificial Sequence Synthetic polypeptide”
QSALNQPRSVSGSPGQSVSISCTGSSSDVGGYEFVSWYQHHPGKAPKLIIYDVDKRPSGVPDRFSGSRSGDTASLTISGLQAEDEADYYCYSSADTWVFGGGTKLTVL

SEQ ID NO: 143 - DNA, 352 bp, Artificial Sequence
  source: /note=“Description of Artificial Sequence Synthetic polynucleotide”
caggtgcagctggtggagtctgggggaggcgtggtccagcctggggggtccctgagattgtcctgtgcagcgtctggattctccttcagtaattatggcatgcactgggtccgccaggctccaggcaaggggctggagtgggtggcacttataccgtctgatggaaattatcaatactatacagactccgtgaagggccgattcaccgtctccagagacaattccaggaacacgttgtatctgcaaatgaagagcctgagagctgaggacacggctagatatcattgtgcccacctcggggggggtttatttgacttctggggccagggcaccctggtcaccgtctcctcag

SEQ ID NO: 144 - DNA, 325 bp, Artificial Sequence
  source: /note=“Description of Artificial Sequence Synthetic polynucleotide”
cagtctgccctgaatcagcctcgctcagtgtccgggtctcctggacagtcagtctccatctcctgcactggctccagcagtgatgttggtggttatgagtttgtctcctggtaccaacaccacccaggcaaagcccccaaactcataatttatgatgtcgataagcggccctcaggggtccctgatcgcttctctggctccaggtctggcgacacggcctccctgaccatctctgggctccaggctgaggatgaggctgattattactgctactcatctgcagacacctgggtcttcggcggagggaccaagctcactgtcctag

SEQ ID NO: 145 - protein, 8 aa, Artificial Sequence
  source: /note=“Description of Artificial Sequence Synthetic peptide”
GGFTSSYY

SEQ ID NO: 146 - protein, 7 aa, Artificial Sequence
  source: /note=“Description of Artificial Sequence Synthetic peptide”
VYYGEST

SEQ ID NO: 147 - protein, 11 aa, Artificial Sequence
  source: /note=“Description of Artificial Sequence Synthetic peptide”
AREVDKRGFDY

SEQ ID NO: 148 - protein, 7 aa, Artificial Sequence
  source: /note=“Description of Artificial Sequence Synthetic peptide”
QSVSGGY

SEQ ID NO: 149 - protein, 3 aa, Artificial Sequence
  source: /note=“Description of Artificial Sequence Synthetic peptide”
GAS

SEQ ID NO: 150 - protein, 9 aa, Artificial Sequence
  source: /note=“Description of Artificial Sequence Synthetic peptide”
QQYGRTPLT

SEQ ID NO: 151 - DNA, 24 bp, Artificial Sequence
  source: /note=“Description of Artificial Sequence Synthetic oligonucleotide”
ggtggcttcaccagtagttattat

SEQ ID NO: 152 - DNA, 21 bp, Artificial Sequence
  source: /note=“Description of Artificial Sequence Synthetic oligonucleotide”
gtgtattacggtgaaagtacc

SEQ ID NO: 153 - DNA, 33 bp, Artificial Sequence
  source: /note=“Description of Artificial Sequence Synthetic oligonucleotide”
gcgagagaagtggataaacggggctttgactac

SEQ ID NO: 154 - DNA, 21 bp, Artificial Sequence
  source: /note=“Description of Artificial Sequence Synthetic oligonucleotide”
cagagtgttagcggcggttac

SEQ ID NO: 155 - DNA, 9 bp, Artificial Sequence
  source: /note=“Description of Artificial Sequence Synthetic oligonucleotide”
ggtgcatcc

SEQ ID NO: 156 - DNA, 27 bp, Artificial Sequence
  source: /note=“Description of Artificial Sequence Synthetic oligonucleotide”
cagcagtatggtaggacaccgctcact

SEQ ID NO: 157 - protein, 117 aa, Artificial Sequence
  source: /note=“Description of Artificial Sequence Synthetic polypeptide”
QVQLQESGPGLVKPSETLSLTCSVSGGFTSSYYWSWIRQAPGKGLEWIGYVYYGESTDYNPSLKSRATISIDTSKNQFSLKLSSVTAADTAVYYCAREVDKRGFDYWGQGALVTVSS

SEQ ID NO: 158 - protein, 108 aa, Artificial Sequence
  source: /note=“Description of Artificial Sequence Synthetic polypeptide”
EIVLTQSPGTLSLSPGERATLSCRASQSVSGGYLAWYQQEPGQAPRLVIYGASSRATGIPDRFSASGSGTDFTLTITRLEPEDFAVYYCQQYGRTPLTFGGGTKVEIK

SEQ ID NO: 159 - DNA, 352 bp, Artificial Sequence
  source: /note=“Description of Artificial Sequence Synthetic polynucleotide”
caggtgcagctgcaggagtcgggcccaggactggtgaagccttcggagaccctgtccctcacctgcagtgtctctggtggcttcaccagtagttattattggagttggatccggcaggcccccgggaagggactggagtggattggctatgtgtattacggtgaaagtaccgattacaacccctccctcaagagtcgagccaccatatcaatagacacgtccaagaaccaattctccctgaagctgagctctgtgaccgctgcggacacggccgtctattattgtgcgagagaagtggataaacggggctttgactactggggccagggagccctggtcaccgtctcctcag

SEQ ID NO: 160 - DNA, 325 bp, Artificial Sequence
  source: /note=“Description of Artificial Sequence Synthetic polynucleotide”
gaaattgtgttgacgcagtctccaggcaccctatctttgtctccaggggaaagagccaccctctcctgcagggccagtcagagtgttagcggcggttacttagcctggtaccagcaggaacctggccaggctcccaggctcgtcatctatggtgcatccagcagggccactggcatcccagacaggttcagtgccagtgggtctgggacagacttcactctcaccatcaccagactggagccagaagattttgcagtgtattactgtcagcagtatggtaggacaccgctcactttcggcggagggaccaaggtggagatcaaac

SEQ ID NO: 161 - protein, 8 aa, Artificial Sequence
  source: /note=“Description of Artificial Sequence Synthetic peptide”
ISYDASSK

SEQ ID NO: 162 - protein, 17 aa, Artificial Sequence
  source: /note=“Description of Artificial Sequence Synthetic peptide”
AKALRYLDWFLSDPFDY

SEQ ID NO: 163 - protein, 7 aa, Artificial Sequence
  source: /note=“Description of Artificial Sequence Synthetic peptide”
QSVSSDF

SEQ ID NO: 164 - protein, 8 aa, Artificial Sequence
  source: /note=“Description of Artificial Sequence Synthetic peptide”
QQYAASPP

SEQ ID NO: 165 - DNA, 24 bp, Artificial Sequence
  source: /note=“Description of Artificial Sequence Synthetic oligonucleotide”
ggattcaccttcagtaactatggc

SEQ ID NO: 166 - DNA, 24 bp, Artificial Sequence
  source: /note=“Description of Artificial Sequence Synthetic oligonucleotide”
atatcttatgatgcaagtagtaaa

SEQ ID NO: 167 - DNA, 51 bp, Artificial Sequence
  source: /note=“Description of Artificial Sequence Synthetic oligonucleotide”
gcgaaagccctacgatatcttgactggttcctctcggaccccttcgactac

SEQ ID NO: 168 - DNA, 21 bp, Artificial Sequence
  source: /note=“Description of Artificial Sequence Synthetic oligonucleotide”
cagagtgttagtagcgacttc

SEQ ID NO: 169 - DNA, 24 bp, Artificial Sequence
  source: /note=“Description of Artificial Sequence Synthetic oligonucleotide”
cagcagtatgctgcctcaccgccc

SEQ ID NO: 170 - protein, 124 aa, Artificial Sequence
  source: /note=“Description of Artificial Sequence Synthetic polypeptide”
QVQLVESGGGVVQPGRSLRLSCAASGFTFSNYGMHWVRQGPGKGLEWVAVISYDASSKYYTDSVQGRFTISRDNSKNTLFLQMNSLRGEDTAVYYCAKALRYLDWFLSDPFDYWGQGTLVTVSS

SEQ ID NO: 171 - protein, 107 aa, Artificial Sequence
  source: /note=“Description of Artificial Sequence Synthetic polypeptide”
EIVLTQSPGTLSLSPGERATLSCRASQSVSSDFLAWYQQKPGQAPRLLIYGASSRATGIPDRFSGSGSGTDFTLTISRLEPEDFAVYYCQQYAASPPFGQGTRLEIK

SEQ ID NO: 172 - DNA, 373 bp, Artificial Sequence
  source: /note=“Description of Artificial Sequence Synthetic polynucleotide”
caggtgcaactggtggagtctgggggaggcgtggtccagcctgggaggtccctcagactctcctgtgcagcctctggattcaccttcagtaactatggcatgcactgggtccgccagggtccaggcaaggggctggagtgggtggcagttatatcttatgatgcaagtagtaaatactatacagactccgtgcagggccgattcaccatctccagagacaattccaagaacacactgtttctgcaaatgaacagcctgagaggtgaagacacggctgtgtattactgtgcgaaagccctacgatatcttgactggttcctctcggaccccttcgactactggggccagggaaccctggtcaccgtctcctcag

SEQ ID NO: 173 - DNA, 322 bp, Artificial Sequence
  source: /note=“Description of Artificial Sequence Synthetic polynucleotide”
gaaattgtgttgacgcagtctccaggcaccctgtctttgtctccaggggaaagagccaccctctcctgcagggccagtcagagtgttagtagcgacttcttagcctggtaccagcagaaacctggccaggctcccaggctcctcatctatggtgcatccagcagggccactggcatcccagacaggttcagtggcagtgggtctgggacagacttcactctcaccatcagccgactggagcctgaagattttgcagtctattactgtcagcagtatgctgcctcaccgcccttcggccaagggacacgactggagattaaac

SEQ ID NO: 174 - protein, 8 aa, Artificial Sequence
  source: /note=“Description of Artificial Sequence Synthetic peptide”
GFTFSSDG

SEQ ID NO: 175 - protein, 8 aa, Artificial Sequence
  source: /note=“Description of Artificial Sequence Synthetic peptide”
ISSDGSTP

SEQ ID NO: 176 - protein, 15 aa, Artificial Sequence
  source: /note=“Description of Artificial Sequence Synthetic peptide”
AKDWALFRWLRTFDH

SEQ ID NO: 177 - protein, 6 aa, Artificial Sequence
  source: /note=“Description of Artificial Sequence Synthetic peptide”
QSVGIN

SEQ ID NO: 178 - protein, 10 aa, Artificial Sequence
  source: /note=“Description of Artificial Sequence Synthetic peptide”
QQYNDWPPWT

SEQ ID NO: 179 - DNA, 24 bp, Artificial Sequence
  source: /note=“Description of Artificial Sequence Synthetic oligonucleotide”
ggattcaccttcagtagcgacggc

SEQ ID NO: 180 - DNA, 24 bp, Artificial Sequence
  source: /note=“Description of Artificial Sequence Synthetic oligonucleotide”
atatcatctgacggaagtactcca

SEQ ID NO: 181 - DNA, 45 bp, Artificial Sequence
  source: /note=“Description of Artificial Sequence Synthetic oligonucleotide”
gccaaagattgggcattatttcggtggctacgaacctttgatcat

SEQ ID NO: 182 - DNA, 18 bp, Artificial Sequence
  source: /note=“Description of Artificial Sequence Synthetic oligonucleotide”
cagagtgttggcatcaat

SEQ ID NO: 183 - DNA, 30 bp, Artificial Sequence
  source: /note=“Description of Artificial Sequence Synthetic oligonucleotide”
caacaatataatgactggcctccgtggacg

SEQ ID NO: 184 - protein, 122 aa, Artificial Sequence
  source: /note=“Description of Artificial Sequence Synthetic polypeptide”
LVELVESGGGVVQPGRSLRLSCAASGFTFSSDGMHWVRQSPGRGLEWVAFISSDGSTPYYADSVKGRFTISRDNSKNTLYLQMNSLRAEDTAMYFCAKDWALFRWLRTFDHWGQGTLVTVSS

SEQ ID NO: 185 - protein, 108 aa, Artificial Sequence
  source: /note=“Description of Artificial Sequence Synthetic polypeptide”
ETVMTQSPATLSVSPGGRATLSCRASQSVGINLAWYQQKPGQAPRLLIYGASTRASGFPARFSGSGSGTEFTLTITSLQSEDFAVYYCQQYNDWPPWTFGQGTKVEIK

SEQ ID NO: 186 - DNA, 367 bp, Artificial Sequence
  source: /note=“Description of Artificial Sequence Synthetic polynucleotide”
ctggtggaactggtggagtctgggggaggcgtggtccagcctgggaggtccctgagactctcctgtgcagcctctggattcaccttcagtagcgacggcatgcactgggtccgccagagtccaggcagggggctggaatgggtggcctttatatcatctgacggaagtactccatactatgctgactccgtgaagggccgattcaccatctccagagacaattccaagaacacactgtatctgcaaatgaacagcctcagagctgaggacacggctatgtacttctgtgccaaagattgggcattatttcggtggctacgaacctttgatcattggggccagggaaccctggtcaccgtctcctcag

SEQ ID NO: 187 - DNA, 325 bp, Artificial Sequence
  source: /note=“Description of Artificial Sequence Synthetic polynucleotide”
gaaacggtgatgacgcagtctccagccaccctgtctgtgtctcctgggggaagagccaccctctcctgcagggccagtcagagtgttggcatcaatttagcctggtaccagcagaaacctggccaggctcccaggctcctcatctatggtgcatccaccagggcctctggtttcccagccaggttcagtggcagtgggtctgggacagagttcactctcaccatcaccagcctgcagtctgaagattttgcagtctattactgtcaacaatataatgactggcctccgtggacgttcggccaagggaccaaggtggagatcaaac

SEQ ID NO: 188 - protein, 8 aa, Artificial Sequence
  source: /note=“Description of Artificial Sequence Synthetic peptide”
GYSFTNYW

SEQ ID NO: 189 - protein, 8 aa, Artificial Sequence
  source: /note=“Description of Artificial Sequence Synthetic peptide”
IYPGDSDI

SEQ ID NO: 190 - protein, 12 aa, Artificial Sequence
  source: /note=“Description of Artificial Sequence Synthetic peptide”
ARHAIRGDGFDY

SEQ ID NO: 191 - protein, 6 aa, Artificial Sequence
  source: /note=“Description of Artificial Sequence Synthetic peptide”
KLGEKY

SEQ ID NO: 192 - protein, 3 aa, Artificial Sequence
  source: /note=“Description of Artificial Sequence Synthetic peptide”
QDT

SEQ ID NO: 193 - protein, 9 aa, Artificial Sequence
  source: /note=“Description of Artificial Sequence Synthetic peptide”
QAWDTNTVI

SEQ ID NO: 194 - DNA, 24 bp, Artificial Sequence
  source: /note=“Description of Artificial Sequence Synthetic oligonucleotide”
ggatacagctttaccaactactgg

SEQ ID NO: 195 - DNA, 24 bp, Artificial Sequence
  source: /note=“Description of Artificial Sequence Synthetic oligonucleotide”
atctatcctggtgactctgatatc

SEQ ID NO: 196 - DNA, 36 bp, Artificial Sequence
  source: /note=“Description of Artificial Sequence Synthetic oligonucleotide”
gcgagacatgcaatacgaggagatgggtttgactac

SEQ ID NO: 197 - DNA, 18 bp, Artificial Sequence
  source: /note=“Description of Artificial Sequence Synthetic oligonucleotide”
aaattgggggaaaaatac

SEQ ID NO: 198 - DNA, 9 bp, Artificial Sequence
  source: /note=“Description of Artificial Sequence Synthetic oligonucleotide”
caagatacg

SEQ ID NO: 199 - DNA, 27 bp, Artificial Sequence
  source: /note=“Description of Artificial Sequence Synthetic oligonucleotide”
caggcgtgggacaccaacactgtgata

SEQ ID NO: 200 - protein, 119 aa, Artificial Sequence
  source: /note=“Description of Artificial Sequence Synthetic polypeptide”
EVQLVQSGAEVKKPGESLKISCQASGYSFTNYWIAWVRQMPGKGLEWMGIIYPGDSDIKYSPSFRGQVTISADKSISNAFLQWRSLRASDTAMYYCARHAIRGDGFDYWGQGTLVTVSS

SEQ ID NO: 201 - protein, 106 aa, Artificial Sequence
  source: /note=“Description of Artificial Sequence Synthetic polypeptide”
SYELTQPPSVSVSPGQTATITCSGDKLGEKYACWYQQKPGQSPVLVMYQDTKRPSGIPERFSGSNSGNTATLTISGTRAMDEADYYCQAWDTNTVIFGGGTKLTVL

SEQ ID NO: 202 - DNA, 358 bp, Artificial Sequence
  source: /note=“Description of Artificial Sequence Synthetic polynucleotide”
gaggtgcagctggtgcagtctggagcagaagtgaaaaagcccggggagtctctgaagatctcctgtcaggcttctggatacagctttaccaactactggatcgcctgggtgcgccagatgcccgggaaaggcctggagtggatgggcatcatctatcctggtgactctgatatcaaatacagcccgtccttccgaggccaggtcaccatctcagccgacaagtccatcagtaatgccttcctccagtggcgaagcctgagggcctcggacaccgccatgtattactgtgcgagacatgcaatacgaggagatgggtttgactactggggccagggaaccctggtcaccgtctcctcag

SEQ ID NO: 203 - DNA, 319 bp, Artificial Sequence
  source: /note=“Description of Artificial Sequence Synthetic polynucleotide”
tcctatgagctgactcagccaccctcagtgtccgtgtccccaggacagacagccaccatcacctgctctggagataaattgggggaaaaatacgcttgctggtatcagcagaagccaggccagtcccctgttttggtcatgtatcaagatacgaagcggccctcagggatccctgagcgattctctggctccaactctgggaacacagccactctgaccatcagcgggacccgggctatggatgaagctgactattactgtcaggcgtgggacaccaacactgtgatattcggcggagggaccaagctgaccgtcctag

SEQ ID NO: 204 - protein, 8 aa, Artificial Sequence
  source: /note=“Description of Artificial Sequence Synthetic peptide”
IYPGDSDT

SEQ ID NO: 205 - protein, 12 aa, Artificial Sequence
  source: /note=“Description of Artificial Sequence Synthetic peptide”
GRHAIRGDGFDY

SEQ ID NO: 206 - DNA, 24 bp, Artificial Sequence
  source: /note=“Description of Artificial Sequence Synthetic oligonucleotide”
atctatcctggtgactctgatacc

SEQ ID NO: 207 - DNA, 36 bp, Artificial Sequence
  source: /note=“Description of Artificial Sequence Synthetic oligonucleotide”
gggagacatgcaatacgaggagatgggtttgactac

SEQ ID NO: 208 - protein, 119 aa, Artificial Sequence
  source: /note=“Description of Artificial Sequence Synthetic polypeptide”
EVQLVQSGAEVKKPGESLKISCQASGYSFTNYWIAWVRQMPGKGLEWMGIIYPGDSDTKYSPSFRGQVTISADKSISTAFLQWRSLRASDTAMYYCGRHAIRGDGFDYWGQGTLVTVSS

SEQ ID NO: 209 - DNA, 358 bp, Artificial Sequence
  source: /note=“Description of Artificial Sequence Synthetic polynucleotide”
gaggtgcagctggtgcagtctggagcagaagtgaaaaagcccggggagtctctgaagatctcctgtcaggcttctggatacagctttaccaactactggatcgcctgggtgcgccagatgcccgggaaaggcctggagtggatgggcatcatctatcctggtgactctgataccaaatacagcccgtccttccgaggccaggtcaccatctcagccgacaagtccatcagtactgccttcctccagtggcgaagcctgagggcctcggacaccgccatgtattactgtgggagacatgcaatacgaggagatgggtttgactactggggccagggaaccctggtcaccgtctcctcag

SEQ ID NO: 210 - protein, 12 aa, Artificial Sequence
  source: /note=“Description of Artificial Sequence Synthetic peptide”
ERHAIRGDGFDY

SEQ ID NO: 211 - DNA, 36 bp, Artificial Sequence
  source: /note=“Description of Artificial Sequence Synthetic oligonucleotide”
gagagacatgcaatacgaggagatgggtttgactac

SEQ ID NO: 212 - protein, 119 aa, Artificial Sequence
  source: /note=“Description of Artificial Sequence Synthetic polypeptide”
EVQLVQSGAEVKKPGESLKISCQASGYSFTNYWIAWVRQMPGKGLEWMGIIYPGDSDTKYSPSFRGQVTISADKSISTAFLQWRSLRASDTAMYYCERHAIRGDGFDYWGQGTLVTVSS

SEQ ID NO: 213 - protein, 106 aa, Artificial Sequence
  source: /note=“Description of Artificial Sequence Synthetic polypeptide”
SYVLTQPPSVSVSPGQTATITCSGDKLGEKYACWYQQKPGQSPVLVMYQDTKRPSGIPERFSGSNSGNTATLTISGTRAMDEADYYCQAWDTNTVIFGGGTKLTVL

SEQ ID NO: 214 - DNA, 358 bp, Artificial Sequence
  source: /note=“Description of Artificial Sequence Synthetic polynucleotide”
gaggtgcagctggtgcagtctggagcagaagtgaaaaagcccggggagtctctgaagatctcctgtcaggcttctggatacagctttaccaactactggatcgcctgggtgcgccagatgcccgggaaaggcctggagtggatgggcatcatctatcctggtgactctgataccaaatacagcccgtccttccgaggccaggtcaccatctcagccgacaagtccatcagtactgccttcctccagtggcgaagcctgagggcctcggacaccgccatgtattactgtgagagacatgcaatacgaggagatgggtttgactactggggccagggaaccctggtcaccgtctcctcag

SEQ ID NO: 215 - DNA, 319 bp, Artificial Sequence
  source: /note=“Description of Artificial Sequence Synthetic polynucleotide”
tcctatgtcctgactcagccaccctcagtgtccgtgtccccaggacagacagccaccatcacctgctctggagataaattgggggaaaaatacgcttgctggtatcagcagaagccaggccagtcccctgttttggtcatgtatcaagatacgaagcggccctcagggatccctgagcgattctctggctccaactctgggaacacagccactctgaccatcagcgggacccgggctatggatgaagctgactattactgtcaggcgtgggacaccaacactgtgatattcggcggagggaccaagctgaccgtcctag

SEQ ID NO: 216 - protein, 8 aa, Artificial Sequence
  source: /note=“Description of Artificial Sequence Synthetic peptide”
GYTFTNYY

SEQ ID NO: 217 - protein, 8 aa, Artificial Sequence
  source: /note=“Description of Artificial Sequence Synthetic peptide”
IHPSSGGT

SEQ ID NO: 218 - protein, 16 aa, Artificial Sequence
  source: /note=“Description of Artificial Sequence Synthetic peptide”
GRAFRILGLSDVFVND

SEQ ID NO: 219 - protein, 6 aa, Artificial Sequence
  source: /note=“Description of Artificial Sequence Synthetic peptide”
QGINNY

SEQ ID NO: 220 - protein, 3 aa, Artificial Sequence
  source: /note=“Description of Artificial Sequence Synthetic peptide”
AAS

SEQ ID NO: 221 - protein, 9 aa, Artificial Sequence
  source: /note=“Description of Artificial Sequence Synthetic peptide”
QKYNSAPFT

SEQ ID NO: 222 - DNA, 24 bp, Artificial Sequence
  source: /note=“Description of Artificial Sequence Synthetic oligonucleotide”
ggatacaccttcaccaactactat

SEQ ID NO: 223 - DNA, 24 bp, Artificial Sequence
  source: /note=“Description of Artificial Sequence Synthetic oligonucleotide”
atccaccctagtagtggtggcaca

SEQ ID NO: 224 - DNA, 48 bp, Artificial Sequence
  source: /note=“Description of Artificial Sequence Synthetic oligonucleotide”
gggagagcctttcggatcttgggactttcggatgtctttgttaatgac

SEQ ID NO: 225 - DNA, 18 bp, Artificial Sequence
  source: /note=“Description of Artificial Sequence Synthetic oligonucleotide”
cagggcattaacaattat

SEQ ID NO: 226 - DNA, 9 bp, Artificial Sequence
  source: /note=“Description of Artificial Sequence Synthetic oligonucleotide”
gctgcatcc

SEQ ID NO: 227 - DNA, 27 bp, Artificial Sequence
  source: /note=“Description of Artificial Sequence Synthetic oligonucleotide”
caaaagtataacagtgcccccttcact

SEQ ID NO: 228 - protein, 123 aa, Artificial Sequence
  source: /note=“Description of Artificial Sequence Synthetic polypeptide”
QVQLVQSGAEVKKPGASVKVSCKASGYTFTNYYIHWVRQAPGQGLEWMGIIHPSSGGTSYAQKFQGRVTMTRDTSTSTVSMDLSSLRSEDTAVYYCGRAFRILGLSDVFVNDWGQGTVVTVSS

SEQ ID NO: 229 - protein, 107 aa, Artificial Sequence
  source: /note=“Description of Artificial Sequence Synthetic polypeptide”
DIQMTQSPSSLSASVGDRVTITCRASQGINNYLAWYQQKPGKVPKLLIYAASTLQSGVPSRFSGSGSGTAFTLTILSLQPEDVATYYCQKYNSAPFTFGPGTKVDIK

SEQ ID NO: 230 - DNA, 370 bp, Artificial Sequence
  source: /note=“Description of Artificial Sequence Synthetic polynucleotide”
caggtgcagttggtgcagtctggggctgaggtgaagaagcctggggcctcagtgaaggtttcctgcaaggcatctggatacaccttcaccaactactatatacactgggtgcgacaggcccctggacaaggacttgagtggatgggaataatccaccctagtagtggtggcacaagctacgcacagaagttccagggcagagtcaccatgaccagggacacgtccacgagcacagtttccatggacctgagcagcctgagatctgaagacacggccgtatattactgtgggagagcctttcggatcttgggactttcggatgtctttgttaatgactggggccagggaactgtggtcaccgtctcctcag

SEQ ID NO: 231 - DNA, 322 bp, Artificial Sequence
  source: /note=“Description of Artificial Sequence Synthetic polynucleotide”
gacatccagatgacccagtctccatcctccctgtctgcatctgtaggagacagagtcaccatcacttgccgggcgagtcagggcattaacaattatttagcctggtatcagcagaaaccagggaaagttcctaagctcctgatctatgctgcatccacattgcaatcaggggtcccatctcggttcagtggcagtggatctgggacagctttcaccctcaccatcctcagcctgcagcctgaagatgttgcaacttattactgtcaaaagtataacagtgcccccttcactttcggccctgggaccaaagtggacatcaaac

SEQ ID NO: 232 - protein, 8 aa, Artificial Sequence
  source: /note=“Description of Artificial Sequence Synthetic peptide”
GFTFTSSA

SEQ ID NO: 233 - protein, 8 aa, Artificial Sequence
  source: /note=“Description of Artificial Sequence Synthetic peptide”
IVLGSGNT

SEQ ID NO: 234 - protein, 14 aa, Artificial Sequence
  source: /note=“Description of Artificial Sequence Synthetic peptide”
AADRGRGGYNVYTY

SEQ ID NO: 235 - protein, 7 aa, Artificial Sequence
  source: /note=“Description of Artificial Sequence Synthetic peptide”
QTISNTY

SEQ ID NO: 236 - protein, 9 aa, Artificial Sequence
  source: /note=“Description of Artificial Sequence Synthetic peptide”
QQNGQSPWT

SEQ ID NO: 237 - DNA, 24 bp, Artificial Sequence
  source: /note=“Description of Artificial Sequence Synthetic oligonucleotide”
ggattcacctttactagctctgct

SEQ ID NO: 238 - DNA, 24 bp, Artificial Sequence
  source: /note=“Description of Artificial Sequence Synthetic oligonucleotide”
atcgtccttggcagcggtaacaca

SEQ ID NO: 239 - DNA, 42 bp, Artificial Sequence
  source: /note=“Description of Artificial Sequence Synthetic oligonucleotide”
gcggcagataggggtagaggtggatacaatgtatacacttac

SEQ ID NO: 240 - DNA, 21 bp, Artificial Sequence
  source: /note=“Description of Artificial Sequence Synthetic oligonucleotide”
cagactattagtaacacctac

SEQ ID NO: 241 - DNA, 27 bp, Artificial Sequence
  source: /note=“Description of Artificial Sequence Synthetic oligonucleotide”
cagcagaatggtcagtcaccttggacg

SEQ ID NO: 242 - protein, 121 aa, Artificial Sequence
  source: /note=“Description of Artificial Sequence Synthetic polypeptide”
QMQLVQSGPQVKKPGTSVKVSCKASGFTFTSSAMQWVRQARGQRPEWIGWIVLGSGNTNYAQKFQERVTLTRDMSTATAYMELSSLRSEDTAVYYCAADRGRGGYNVYTYWGQGTLVAVSS

SEQ ID NO: 243 - protein, 108 aa, Artificial Sequence
  source: /note=“Description of Artificial Sequence Synthetic polypeptide”
EIVMTQSPGTLSLSPGERATLSCRASQTISNTYVAWYQQKPGQAPRLLIYGASSRATGIPDRFSGSGSGTDFTLTIRRLEPEDFAVYYCQQNGQSPWTFGQGTNVEIK

SEQ ID NO: 244 - DNA, 364 bp, Artificial Sequence
  source: /note=“Description of Artificial Sequence Synthetic polynucleotide”
caaatgcagctggtgcagtctgggcctcaagtgaagaagcctgggacctcagtgaaggtctcctgcaaggcttctggattcacctttactagctctgctatgcagtgggtgcggcaggctcgtggacagcgccctgagtggataggatggatcgtccttggcagcggtaacacaaactacgcacagaagttccaggaaagagtcacccttaccagggacatgtccactgctacagcctacatggaactgagcagcctgagatccgaggacacggccgtgtattactgtgcggcagataggggtagaggtggatacaatgtatacacttactggggccaggggaccctggtcgccgtctcctcag

SEQ ID NO: 245 - DNA, 325 bp, Artificial Sequence
  source: /note=“Description of Artificial Sequence Synthetic polynucleotide”
gaaattgtgatgacgcagtctccaggcaccctgtctttgtctccaggggaaagagccaccctctcctgcagggccagtcagactattagtaacacctacgtggcctggtaccagcagaaacctggccaggctcccaggctcctcatctatggtgcatccagcagggccactggcatcccagacaggttcagtggcagtgggtctgggacagacttcactctcaccatccgcagactggagcctgaagattttgcagtgtattactgtcagcagaatggtcagtcaccttggacgttcggccaagggaccaacgtggaaatcaaac

SEQ ID NO: 246 - protein, 8 aa, Artificial Sequence
  source: /note=“Description of Artificial Sequence Synthetic peptide”
GYTFTGYY

SEQ ID NO: 247 - protein, 8 aa, Artificial Sequence
  source: /note=“Description of Artificial Sequence Synthetic peptide”
INPMTGAT

SEQ ID NO: 248 - protein, 18 aa, Artificial Sequence
  source: /note=“Description of Artificial Sequence Synthetic peptide”
ARGGPTSTRITGKRHFDL

SEQ ID NO: 249 - protein, 9 aa, Artificial Sequence
  source: /note=“Description of Artificial Sequence Synthetic peptide”
ISDVGAYNS

SEQ ID NO: 250 - protein, 3 aa, Artificial Sequence
  source: /note=“Description of Artificial Sequence Synthetic peptide”
DVT

SEQ ID NO: 251 - protein, 10 aa, Artificial Sequence
  source: /note=“Description of Artificial Sequence Synthetic peptide”
SSYTTSDTYV

SEQ ID NO: 252 - DNA, 24 bp, Artificial Sequence
  source: /note=“Description of Artificial Sequence Synthetic oligonucleotide”
ggatacaccttcaccggctactat

SEQ ID NO: 253 - DNA, 24 bp, Artificial Sequence
  source: /note=“Description of Artificial Sequence Synthetic oligonucleotide”
atcaaccctatgactggagccaca

SEQ ID NO: 254 - DNA, 54 bp, Artificial Sequence
  source: /note=“Description of Artificial Sequence Synthetic oligonucleotide”
gcgagaggaggtcctaccagtacccgaataacagggaaacggcacttcgatctc

SEQ ID NO: 255 - DNA, 27 bp, Artificial Sequence
  source: /note=“Description of Artificial Sequence Synthetic oligonucleotide”
atcagtgacgttggtgcttataactct

SEQ ID NO: 256 - DNA, 9 bp, Artificial Sequence
  source: /note=“Description of Artificial Sequence Synthetic oligonucleotide”
gacgtcact

SEQ ID NO: 257 - DNA, 30 bp, Artificial Sequence
  source: /note=“Description of Artificial Sequence Synthetic oligonucleotide”
agctcatatacaaccagtgacacttatgtc

SEQ ID NO: 258 - protein, 125 aa, Artificial Sequence
  source: /note=“Description of Artificial Sequence Synthetic polypeptide”
RAQLVQSAAEMKNPGASVKVSCEASGYTFTGYYVHWMRQAPGQGLEWMGWINPMTGATKSPQKFQGRVTMTRDTSTTATHIELTRLRSDDSAVFFCARGGPTSTRITGKRHFDLWGRGTLITVAS

SEQ ID NO: 259 - protein, 110 aa, Artificial Sequence
  source: /note=“Description of Artificial Sequence Synthetic polypeptide”
QSALTQPASVSGSPGESITISCTGTISDVGAYNSVSWYQQHSGTAPELIIYDVTNRPAGVSSRFSGSKSGNTASLTISWLQSEDEAEYYCSSYTTSDTYVFGSGTQVTVL

SEQ ID NO: 260 - DNA, 376 bp, Artificial Sequence
  source: /note=“Description of Artificial Sequence Synthetic polynucleotide”
cgggcgcagttggtgcagtctgcggctgagatgaagaaccctggggcctcagtgaaggtctcctgcgaggcttctggatacaccttcaccggctactatgtacactggatgcgacaggcccccggacaaggactagagtggatgggatggatcaaccctatgactggagccacaaagtctccacagaagtttcagggcagggtcaccatgaccagggacacttccaccaccgcaacccacatagaactgactaggctgagatctgacgacagtgccgtctttttctgtgcgagaggaggtcctaccagtacccgaataacagggaaacggcacttcgatctctggggccgcggcaccctgatcactgtcgcctcag

SEQ ID NO: 261 - DNA, 331 bp, Artificial Sequence
  source: /note=“Description of Artificial Sequence Synthetic polynucleotide”
cagtctgccctgactcagcctgcctccgtgtctgggtctcctggagagtcgatcaccatctcctgcactggaaccatcagtgacgttggtgcttataactctgtctcctggtaccaacaacactcaggcacagcccccgaactcatcatttatgacgtcactaatcggcccgcaggggtttcgagtcgcttctctggctccaagtctggcaacacggcctccctgaccatctcttggctccagtctgaggacgaggctgaatattattgcagctcatatacaaccagtgacacttatgtcttcggaagtgggacccaagtcaccgtcctaa

SEQ ID NO: 262 - protein, 8 aa, Artificial Sequence
  source: /note=“Description of Artificial Sequence Synthetic peptide”
GFTVSTTY

SEQ ID NO: 263 - protein, 12 aa, Artificial Sequence
  source: /note=“Description of Artificial Sequence Synthetic peptide”
IHTGGIFGVGGT

SEQ ID NO: 264 - protein, 13 aa, Artificial Sequence
  source: /note=“Description of Artificial Sequence Synthetic peptide”
AREHRGTIDAFDA

SEQ ID NO: 265 - protein, 6 aa, Artificial Sequence
  source: /note=“Description of Artificial Sequence Synthetic peptide”
QNIRNY

SEQ ID NO: 266 - protein, 3 aa, Artificial Sequence
  source: /note=“Description of Artificial Sequence Synthetic peptide”
TTS

SEQ ID NO: 267 - protein, 8 aa, Artificial Sequence
  source: /note=“Description of Artificial Sequence Synthetic peptide”
QQSYDGWT

SEQ ID NO: 268 - DNA, 24 bp, Artificial Sequence
  source: /note=“Description of Artificial Sequence Synthetic oligonucleotide”
ggattcaccgtcagtaccacctac

SEQ ID NO: 269 - DNA, 36 bp, Artificial Sequence
  source: /note=“Description of Artificial Sequence Synthetic oligonucleotide”
attcataccggtggcatttttggcgttggcggtaca

SEQ ID NO: 270 - DNA, 39 bp, Artificial Sequence
  source: /note=“Description of Artificial Sequence Synthetic oligonucleotide”
gcgagggaacatcggggaactatcgatgcttttgatgcc

SEQ ID NO: 271 - DNA, 18 bp, Artificial Sequence
  source: /note=“Description of Artificial Sequence Synthetic oligonucleotide”
cagaacattcgaaattat

SEQ ID NO: 272 - DNA, 9 bp, Artificial Sequence
  source: /note=“Description of Artificial Sequence Synthetic oligonucleotide”
actacatcc

SEQ ID NO: 273 - DNA, 24 bp, Artificial Sequence
  source: /note=“Description of Artificial Sequence Synthetic oligonucleotide”
caacagagttacgatgggtggacg

SEQ ID NO: 274 - protein, 124 aa, Artificial Sequence
  source: /note=“Description of Artificial Sequence Synthetic polypeptide”
EVRLEESGGGLVQPGGSLRLSCAASGFTVSTTYMAWVRQAPGKGLEWVSLIHTGGIFGVGGTSYADSVKGRFTISRDTSKNTVSLQMSSLRVEDTAIYFCAREHRGTIDAFDAWGQGTVVIVSS

SEQ ID NO: 275 - protein, 106 aa, Artificial Sequence
  source: /note=“Description of Artificial Sequence Synthetic polypeptide”
DIHMTQSPSSLSASVGDRVTITCRASQNIRNYLNWYQHKPGKAPKLLIYTTSRLQSGVPSRFSGSGSGTDFTLTVNSLQPEDFASYYCQQSYDGWTFGQGTKVEMK

SEQ ID NO: 276 - DNA, 373 bp, Artificial Sequence
  source: /note=“Description of Artificial Sequence Synthetic polynucleotide”
gaggtgcgactggaggagtctgggggaggcttggtccagcctggggggtccctgagactctcctgtgcagcctctggattcaccgtcagtaccacctacatggcctgggtccgccaggctccagggaaggggctggaatgggtctcacttattcataccggtggcatttttggcgttggcggtacatcctacgcagactccgtgaagggcagattcaccatctccagagacacttccaagaacacagtgtctcttcaaatgagcagcctgagagtcgaggacacggccatctatttctgtgcgagggaacatcggggaactatcgatgcttttgatgcctggggccaagggacagtggtcatcgtctcttcag

SEQ ID NO: 277 - DNA, 319 bp, Artificial Sequence
  source: /note=“Description of Artificial Sequence Synthetic polynucleotide”
gacatccacatgacccagtctccatcctccctgtctgcatctgttggagacagagtcaccatcacttgccgggcaagtcagaacattcgaaattatttaaattggtatcaacataaaccagggaaagcccctaaactcctgatctatactacatcccgtctgcaaagtggggtcccatcaaggttcagtggcagtggatctgggacagatttcactctcaccgtcaacagcctgcaaccagaagactttgcaagttactactgtcaacagagttacgatgggtggacgttcggccaggggaccaaggtggaaatgaaac

SEQ ID NO: 278 - protein, 8 aa, Artificial Sequence
  source: /note=“Description of Artificial Sequence Synthetic peptide”
GFTFSSYE

SEQ ID NO: 279 - protein, 8 aa, Artificial Sequence
  source: /note=“Description of Artificial Sequence Synthetic peptide”
IDFTGSTI

SEQ ID NO: 280 - protein, 16 aa, Artificial Sequence
  source: /note=“Description of Artificial Sequence Synthetic peptide”
VRDAGRWGTSWYYFDY

SEQ ID NO: 281 - protein, 9 aa, Artificial Sequence
  source: /note=“Description of Artificial Sequence Synthetic peptide”
SSNIGAGYD

SEQ ID NO: 282 - protein, 3 aa, Artificial Sequence
  source: /note=“Description of Artificial Sequence Synthetic peptide”
GNN

SEQ ID NO: 283 - protein, 11 aa, Artificial Sequence
  source: /note=“Description of Artificial Sequence Synthetic peptide”
QSYDSSLNGWV

SEQ ID NO: 284 - DNA, 24 bp, Artificial Sequence
  source: /note=“Description of Artificial Sequence Synthetic oligonucleotide”
ggattcactttcagtagctatgag

SEQ ID NO: 285 - DNA, 24 bp, Artificial Sequence
  source: /note=“Description of Artificial Sequence Synthetic oligonucleotide”
attgattttactggctcaaccatc

SEQ ID NO: 286 - DNA, 48 bp, Artificial Sequence
  source: /note=“Description of Artificial Sequence Synthetic oligonucleotide”
gtgagagatgcgggccgttggggcaccagttggtactactttgactat

SEQ ID NO: 287 - DNA, 27 bp, Artificial Sequence
  source: /note=“Description of Artificial Sequence Synthetic oligonucleotide”
agctccaacatcggggcaggttatgat

SEQ ID NO: 288 - DNA, 9 bp, Artificial Sequence
  source: /note=“Description of Artificial Sequence Synthetic oligonucleotide”
ggtaacaac

SEQ ID NO: 289 - DNA, 33 bp, Artificial Sequence
  source: /note=“Description of Artificial Sequence Synthetic oligonucleotide”
cagtcgtatgacagcagcctgaatggttgggtg

SEQ ID NO: 290 - protein, 123 aa, Artificial Sequence
  source: /note=“Description of Artificial Sequence Synthetic polypeptide”
AVQLVESGGGLAQPGRSLRLSCKVSGFTFSSYEMNWVRQAPGKGLEWIAYIDFTGSTIYYADSVKGRFTISRDTARNSLYLQMNKLRVEDTAVYYCVRDAGRWGTSWYYFDYWGQGTLVTVSS

SEQ ID NO: 291 - protein, 111 aa, Artificial Sequence
  source: /note=“Description of Artificial Sequence Synthetic polypeptide”
QSVLTQPPSVSGAPGQRVTISCTGLSSNIGAGYDIHWYQQIPGKAPKLLIYGNNNRPSGVPDRFSGSKSGTSVSLAITGLQAEDEADYYCQSYDSSLNGWVFGGGTRLTVL

SEQ ID NO: 292 - DNA, 370 bp, Artificial Sequence
  source: /note=“Description of Artificial Sequence Synthetic polynucleotide”
gcggtgcagctggtggagtctgggggcggcttggcacagcctggacggtccctgaggctctcgtgtaaagtgtctggattcactttcagtagctatgagatgaactgggtccgccaggctccagggaaggggctggagtggattgcatacattgattttactggctcaaccatctactacgcagactctgtgaagggacgattcaccatttccagagacaccgccaggaactcactctatctgcagatgaacaaattgagagtcgaggacacggctgtttattactgtgtgagagatgcgggccgttggggcaccagttggtactactttgactattggggccagggaaccctggtcaccgtctcctcag

SEQ ID NO: 293 - DNA, 334 bp, Artificial Sequence
  source: /note=“Description of Artificial Sequence Synthetic polynucleotide”
cagtctgtgctgacgcagccgccctcagtgtctggggccccagggcagagggtcaccatctcctgcactgggctcagctccaacatcggggcaggttatgatatacactggtatcagcagattccaggaaaagcccccaaactcctcatctatggtaacaacaatcggccctcaggggtccctgaccgattctctggctctaagtctggcacctcagtctccctggccatcactgggctccaggctgaggatgaggctgattattactgccagtcgtatgacagcagcctgaatggttgggtgttcggcggagggaccaggttgaccgtcctaa

SEQ ID NO: 294 - protein, 123 aa, Artificial Sequence
  source: /note=“Description of Artificial Sequence Synthetic polypeptide”
AVQLVESGGDLAQPGRSLRLSCKVSGFTFSSYEMNWVRQAPGKGLEWIAYIDFTGSTIYYADSVKGRFTISRDTARNSLYLQMNKLRVEDTAVYYCVRDAGRWGTSWYYFDYWGQGTLVTVSS

SEQ ID NO: 295 - DNA, 370 bp, Artificial Sequence
  source: /note=“Description of Artificial Sequence Synthetic polynucleotide”
gcggtgcagctggtggagtctgggggcgacttggcacagcctggacggtccctgaggctctcgtgtaaagtgtctggattcactttcagtagctatgagatgaactgggtccgccaggctccagggaaggggctggagtggattgcatacattgattttactggctcaaccatctactacgcagactctgtgaagggacgattcaccatttccagagacaccgccaggaactcactctatctgcagatgaacaaattgagagtcgaggacacggctgtttattactgtgtgagagatgcgggccgttggggcaccagttggtactactttgactattggggccagggaaccctggtcaccgtctcctcag

SEQ ID NO: 296 - protein, 8 aa, Artificial Sequence
  source: /note=“Description of Artificial Sequence Synthetic peptide”
GFTFSSHE

SEQ ID NO: 297 - protein, 8 aa, Artificial Sequence
  source: /note=“Description of Artificial Sequence Synthetic peptide”
IDFTGSII

SEQ ID NO: 298 - protein, 16 aa, Artificial Sequence
  source: /note=“Description of Artificial Sequence Synthetic peptide”
ARDGGRWGTSWYYFDY

SEQ ID NO: 299 - protein, 9 aa, Artificial Sequence
  source: /note=“Description of Artificial Sequence Synthetic peptide”
SSNFGAGYD

SEQ ID NO: 300 - protein, 2 aa, Artificial Sequence
  source: /note=“Description of Artificial Sequence Synthetic peptide”
GS

SEQ ID NO: 301 - protein, 11 aa, Artificial Sequence
  source: /note=“Description of Artificial Sequence Synthetic peptide”
QSYDSSLSAWV

SEQ ID NO: 302 - DNA, 24 bp, Artificial Sequence
  source: /note=“Description of Artificial Sequence Synthetic oligonucleotide”
ggattcaccttcagttctcatgag

SEQ ID NO: 303 - DNA, 24 bp, Artificial Sequence
  source: /note=“Description of Artificial Sequence Synthetic oligonucleotide”
attgattttactggcagtattata

SEQ ID NO: 304 - DNA, 48 bp, Artificial Sequence
  source: /note=“Description of Artificial Sequence Synthetic oligonucleotide”
gcgagagatgggggtcgttggggcaccagttggtactactttgactac

SEQ ID NO: 305 - DNA, 27 bp, Artificial Sequence
  source: /note=“Description of Artificial Sequence Synthetic oligonucleotide”
agttccaacttcggggcaggttatgat

SEQ ID NO: 306 - DNA, 6 bp, Artificial Sequence
  source: /note=“Description of Artificial Sequence Synthetic oligonucleotide”
ggtagc

SEQ ID NO: 307 - DNA, 33 bp, Artificial Sequence
  source: /note=“Description of Artificial Sequence Synthetic oligonucleotide”
cagtcctatgacagcagcctgagcgcttgggtg

SEQ ID NO: 308 - protein, 123 aa, Artificial Sequence
  source: /note=“Description of Artificial Sequence Synthetic polypeptide”
AVQLVESGGGLVRPGGSLRLSCAASGFTFSSHEMHWVRQAPGKGLEWLSYIDFTGSIIYYADSVRGRFTISRDNTKKSLFLQMNSLRDEDTALYYCARDGGRWGTSWYYFDYWGQGVLVTVSS

SEQ ID NO: 309 - protein, 110 aa, Artificial Sequence
  source: /note=“Description of Artificial Sequence Synthetic polypeptide”
QSVLTQPPSVSGAPGQRVTITCTGSSSNFGAGYDGHWYQQLPGTAPKLLIYGSNRPSGVPDRFSGSKSGTSVSLAITGLQADDEADYYCQSYDSSLSAWVFGGGTKLTVL

SEQ ID NO: 310 - DNA, 370 bp, Artificial Sequence
  source: /note=“Description of Artificial Sequence Synthetic polynucleotide”
gcggtgcagctggtggagtctgggggaggcttggtacggcctggagggtccctgagactctcctgtgcagcctctggattcaccttcagttctcatgagatgcactgggtccgccaggctccagggaaggggctggaatggctttcatacattgattttactggcagtattatatactacgcagactctgtgaggggtcggttcaccatctccagagacaacaccaaaaagtcactgtttctgcaaatgaacagcctgagagacgaggatacggctctttattactgtgcgagagatgggggtcgttggggcaccagttggtactactttgactactggggccagggagtcctggtcaccgtctcctcag

SEQ ID NO: 311 - DNA, 331 bp, Artificial Sequence
  source: /note=“Description of Artificial Sequence Synthetic polynucleotide”
cagtctgtgctgacgcagccgccctcagtgtctggggccccagggcagagggtcaccataacctgcactgggagcagttccaacttcggggcaggttatgatggacactggtaccagcaacttccaggaacagcccccaaactcctcatctatggtagcaatcggccctcaggggtccctgaccgattctctggctccaagtctggcacctcagtctccctggccatcactgggctccaggctgacgatgaggctgattattactgccagtcctatgacagcagcctgagcgcttgggtgttcggcggagggaccaagctgaccgtcctac

SEQ ID NO: 312 - protein, 8 aa, Artificial Sequence
  source: /note=“Description of Artificial Sequence Synthetic peptide”
IDFTGSSI

SEQ ID NO: 313 - DNA, 24 bp, Artificial Sequence
  source: /note=“Description of Artificial Sequence Synthetic oligonucleotide”
attgattttactggcagtagtata

SEQ ID NO: 314 - protein, 123 aa, Artificial Sequence
  source: /note=“Description of Artificial Sequence Synthetic polypeptide”
AVQLVESGGGLVRPGGSLRLSCAASGFTFSSHEMHWVRQAPGKGLEWLSYIDFTGSSIYYADSVRGRFTISRDNTKKSLFLQMNSLRDEDTALYYCARDGGRWGTSWYYFDYWGQGVLVTVSS

SEQ ID NO: 315 - DNA, 370 bp, Artificial Sequence
  source: /note=“Description of Artificial Sequence Synthetic polynucleotide”
gcggtgcagctggtggagtctgggggaggcttggtacggcctggagggtccctgagactctcctgtgcagcctctggattcaccttcagttctcatgagatgcactgggtccgccaggctccagggaaggggctggaatggctttcatacattgattttactggcagtagtatatactacgcagactctgtgaggggtcggttcaccatctccagagacaataccaaaaagtcactgtttctgcaaatgaacagcctgagagacgaggatacggctctttattactgtgcgagagatgggggtcgttggggcaccagttggtactactttgactactggggccagggagtcctggtcaccgtctcctcag

SEQ ID NO: 316 - protein, 8 aa, Artificial Sequence
  source: /note=“Description of Artificial Sequence Synthetic peptide”
GYTFTDYY

SEQ ID NO: 317 - protein, 8 aa, Artificial Sequence
  source: /note=“Description of Artificial Sequence Synthetic peptide”
FNPNSGGT

SEQ ID NO: 318 - protein, 23 aa, Artificial Sequence
  source: /note=“Description of Artificial Sequence Synthetic peptide”
AKDSAKTASAYYGLNFFYYGMDV

SEQ ID NO: 319 - protein, 8 aa, Artificial Sequence
  source: /note=“Description of Artificial Sequence Synthetic peptide”
SSNIGKNY

SEQ ID NO: 320 - protein, 3 aa, Artificial Sequence
  source: /note=“Description of Artificial Sequence Synthetic peptide”
KNN

SEQ ID NO: 321 - protein, 11 aa, Artificial Sequence
  source: /note=“Description of Artificial Sequence Synthetic peptide”
SAWDGSLSRPL

SEQ ID NO: 322 - DNA, 24 bp, Artificial Sequence
  source: /note=“Description of Artificial Sequence Synthetic oligonucleotide”
ggatacaccttcaccgactactat

SEQ ID NO: 323 - DNA, 24 bp, Artificial Sequence
  source: /note=“Description of Artificial Sequence Synthetic oligonucleotide”
ttcaaccctaacagtggtggcaca

SEQ ID NO: 324 - DNA, 69 bp, Artificial Sequence
  source: /note=“Description of Artificial Sequence Synthetic oligonucleotide”
gcgaaagattccgcgaaaactgcgagtgcttattatggactgaacttcttctactacggtatggacgtc

SEQ ID NO: 325 - DNA, 24 bp, Artificial Sequence
  source: /note=“Description of Artificial Sequence Synthetic oligonucleotide”
agttccaacatcggaaagaattat

SEQ ID NO: 326 - DNA, 9 bp, Artificial Sequence
  source: /note=“Description of Artificial Sequence Synthetic oligonucleotide”
aagaataat

SEQ ID NO: 327 - DNA, 33 bp, Artificial Sequence
  source: /note=“Description of Artificial Sequence Synthetic oligonucleotide”
tcagcgtgggatggcagcctgagtcgtccacta

SEQ ID NO: 328 - protein, 130 aa, Artificial Sequence
  source: /note=“Description of Artificial Sequence Synthetic polypeptide”
QVQLVQSGAEVKNPGASVKVSCKASGYTFTDYYIHWVRQAPGQGLEWMGWFNPNSGGTNFVQNFQGRVTMTRDTSISTAYMELSRLRSDDTAMYYCAKDSAKTASAYYGLNFFYYGMDVWGQGTTVTVSS

SEQ ID NO: 329 - protein, 110 aa, Artificial Sequence
  source: /note=“Description of Artificial Sequence Synthetic polypeptide”
QSVLSQPPSASGTPGQRVTISCSGSSSNIGKNYVYWYQQVPGTAPKLLMFKNNQRPSGVPDRFSGSKSGTSASLAISGLRSEDEADYYCSAWDGSLSRPLFGGGTKVTVL

SEQ ID NO: 330 - DNA, 391 bp, Artificial Sequence
  source: /note=“Description of Artificial Sequence Synthetic polynucleotide”
caggtgcagctggtgcagtctggggctgaagtgaagaaccctggggcctcagtgaaggtctcctgcaaggcttctggatacaccttcaccgactactatatacactgggtgcgacaggcccctggacaaggacttgagtggatgggctggttcaaccctaacagtggtggcacaaactttgtacagaactttcagggcagggtcaccatgaccagggacacgtccatcagcacagcctacatggagctcagcaggctgagatctgacgacacggccatgtattactgtgcgaaagattccgcgaaaactgcgagtgcttattatggactgaacttcttctactacggtatggacgtctggggccaagggaccacggtcaccgtctcctcag

SEQ ID NO: 331 - DNA, 331 bp, Artificial Sequence
  source: /note=“Description of Artificial Sequence Synthetic polynucleotide”
cagtctgtactgagtcagccaccctcagcatctgggacccccgggcagagggtcaccatctcttgttctggaagcagttccaacatcggaaagaattatgtatattggtaccagcaggtcccaggaacggcccccaaactcctcatgtttaagaataatcagcgaccctcaggggtccctgaccgattctctggctccaagtctggcacctctgcctccctggccatcagtgggctccggtccgaggatgaggctgattattattgttcagcgtgggatggcagcctgagtcgtccactattcggcggagggaccaaggtgaccgtcctag

SEQ ID NO: 332 - protein, 23 aa, Artificial Sequence
  source: /note=“Description of Artificial Sequence Synthetic peptide”
ARDSAKTASAYYGLNFFYYGMDV

SEQ ID NO: 333 - DNA, 69 bp, Artificial Sequence
  source: /note=“Description of Artificial Sequence Synthetic oligonucleotide”
gcgagagattccgcgaaaactgcgagtgcttattatggactgaacttcttctactacggtatggacgtc

SEQ ID NO: 334 - protein, 130 aa, Artificial Sequence
  source: /note=“Description of Artificial Sequence Synthetic polypeptide”
QVQLVQSGAEVKNPGASVKVSCKASGYTFTDYYIHWVRQAPGQGLEWMGWFNPNSGGTNFVQNFQGRVTMTRDTSISTAYMELSRLRSDDTAMYYCARDSAKTASAYYGLNFFYYGMDVWGQGTTVTVSS

SEQ ID NO: 335 - DNA, 391 bp, Artificial Sequence
  source: /note=“Description of Artificial Sequence Synthetic polynucleotide”
caggtgcagctggtgcagtctggggctgaagtgaagaaccctggggcctcagtgaaggtctcctgcaaggcttctggatacaccttcaccgactactatatacactgggtgcgacaggcccctggacaaggacttgagtggatgggctggttcaaccctaacagtggtggcacaaactttgtacagaactttcagggcagggtcaccatgaccagggacacgtccatcagcacagcctacatggagctcagcaggctgagatctgacgacacggccatgtattactgtgcgagagattccgcgaaaactgcgagtgcttattatggactgaacttcttctactacggtatggacgtctggggccaagggaccacggtcaccgtctcctcag

SEQ ID NO: 336 - protein, 8 aa, Artificial Sequence
  source: /note=“Description of Artificial Sequence Synthetic peptide”
GFRFNEFN

SEQ ID NO: 337 - protein, 8 aa, Artificial Sequence
  source: /note=“Description of Artificial Sequence Synthetic peptide”
ISIDGRHK

SEQ ID NO: 338 - protein, 16 aa, Artificial Sequence
  source: /note=“Description of Artificial Sequence Synthetic peptide”
VTDGKAVDGFSGILEF

SEQ ID NO: 339 - protein, 6 aa, Artificial Sequence
  source: /note=“Description of Artificial Sequence Synthetic peptide”
QSVGGY

SEQ ID NO: 340 - protein, 3 aa, Artificial Sequence
  source: /note=“Description of Artificial Sequence Synthetic peptide”
DAS

SEQ ID NO: 341 - protein, 10 aa, Artificial Sequence
  source: /note=“Description of Artificial Sequence Synthetic peptide”
QQRNNWPPLT

SEQ ID NO: 342 - DNA, 24 bp, Artificial Sequence
  source: /note=“Description of Artificial Sequence Synthetic oligonucleotide”
ggattcaggttcaatgaatttaat

SEQ ID NO: 343 - DNA, 24 bp, Artificial Sequence
  source: /note=“Description of Artificial Sequence Synthetic oligonucleotide”
atctcaattgatgggagacacaaa

SEQ ID NO: 344 - DNA, 48 bp, Artificial Sequence
  source: /note=“Description of Artificial Sequence Synthetic oligonucleotide”
gtgacagatgggaaagcagtggatgggttttccggaattttagagttc

SEQ ID NO: 345 - DNA, 18 bp, Artificial Sequence
  source: /note=“Description of Artificial Sequence Synthetic oligonucleotide”
cagagtgttggcggctac

SEQ ID NO: 346 - DNA, 9 bp, Artificial Sequence
  source: /note=“Description of Artificial Sequence Synthetic oligonucleotide”
gatgcatcc

SEQ ID NO: 347 - DNA, 30 bp, Artificial Sequence
  source: /note=“Description of Artificial Sequence Synthetic oligonucleotide”
cagcagcgtaacaactggccaccactcact

SEQ ID NO: 348 - protein, 123 aa, Artificial Sequence
  source: /note=“Description of Artificial Sequence Synthetic polypeptide”
QVQLVESGGGVVQPGRSLRLSCAASGFRFNEFNMHWVRQAPGKGLEWVAVISIDGRHKYNADSVEGRFTISRDNSRNTLYLQMNSLRVEDTALYYCVTDGKAVDGFSGILEFWGQGTPVTVST

SEQ ID NO: 349 - protein, 108 aa, Artificial Sequence
  source: /note=“Description of Artificial Sequence Synthetic polypeptide”
EIVLTQSPATLSLSPGERATLSCWASQSVGGYLAWYQQKPGQAPRLLIYDASIRATGIPARFSGSGSGTHFTLTINSLEPEDFAVYYCQQRNNWPPLTFGGGTKVEIK

SEQ ID NO: 350 - DNA, 370 bp, Artificial Sequence
  source: /note=“Description of Artificial Sequence Synthetic polynucleotide”
caggtgcaactggtggagtctgggggaggcgtggtccagcctgggaggtccctgagactctcctgtgcagcctctggattcaggttcaatgaatttaatatgcactgggtccgccaggctccaggcaagggcctggagtgggtggcagttatctcaattgatgggagacacaaatacaacgcagactccgtggagggccgattcaccatctccagagacaattccagaaacactctttatctgcaaatgaacagcctgagagttgaggacacggctctttattactgtgtgacagatgggaaagcagtggatgggttttccggaattttagagttctggggccagggaaccccagtcaccgtctccacag

SEQ ID NO: 351 - DNA, 325 bp, Artificial Sequence
  source: /note=“Description of Artificial Sequence Synthetic polynucleotide”
gaaattgtgttgacacagtctccggccaccctgtctttgtctccaggggagagagccaccctctcctgctgggccagtcagagtgttggcggctacttagcctggtaccaacaaaaacctggccaggctcccaggctcctcatctatgatgcatccatcagggccactggcatcccagccaggttcagtggcagtgggtctgggacacacttcactctcaccatcaatagcctcgagcctgaagattttgccgtttattactgtcagcagcgtaacaactggccaccactcactttcggcggagggaccaaggtggagatcaaac

SEQ ID NO: 352 - protein, 8 aa, Artificial Sequence
  source: /note=“Description of Artificial Sequence Synthetic peptide”
GFSFSNFE

SEQ ID NO: 353 - DNA, 24 bp, Artificial Sequence
  source: /note=“Description of Artificial Sequence Synthetic oligonucleotide”
ggattcagtttcagtaactttgag

SEQ ID NO: 354 - DNA, 24 bp, Artificial Sequence
  source: /note=“Description of Artificial Sequence Synthetic oligonucleotide”
attgattttactggctctaccatc

SEQ ID NO: 355 - DNA, 48 bp, Artificial Sequence
  source: /note=“Description of Artificial Sequence Synthetic oligonucleotide”
gtgagagatgcgggccgttggggcaccagttggtactattttgactat

SEQ ID NO: 356 - DNA, 33 bp, Artificial Sequence
  source: /note=“Description of Artificial Sequence Synthetic oligonucleotide”
cagtcatatgacagcagcctgaatggttgggtg

SEQ ID NO: 357 - protein, 123 aa, Artificial Sequence
  source: /note=“Description of Artificial Sequence Synthetic polypeptide”
AVQLVESGGGLAQPGRSLRLSCKVSGFSFSNFEMNWVRQAPGKGLEWIAYIDFTGSTIYYSDSVKGRFTISRDTARNSLYLQMNKLRVEDTAVYYCVRDAGRWGTSWYYFDYWGQGTLVTVSS

SEQ ID NO: 358 - DNA, 370 bp, Artificial Sequence
  source: /note=“Description of Artificial Sequence Synthetic polynucleotide”
gcggtgcagctggtggaatccgggggcggcttggcacagcctggacggtccctgaggctctcgtgtaaagtgtccggattcagtttcagtaactttgagatgaactgggtccgccaggctccagggaaggggctggagtggattgcatatattgattttactggctctaccatctactactcagactctgtgaagggacggtttaccatttccagagacaccgccaggaactcactctatctgcagatgaacaaattgagagtcgaggacacggctgtttattactgtgtgagagatgcgggccgttggggcaccagttggtactattttgactattggggccagggcaccctggtcaccgtctcctcag

SEQ ID NO: 359 - DNA, 334 bp, Artificial Sequence
  source: /note=“Description of Artificial Sequence Synthetic polynucleotide”
cagtctgtgctgacgcagccgccctcagtgtctggggccccagggcagagggtcaccatctcctgcactgggctcagctccaacatcggggcaggttatgatatacactggtatcagcagattccaggaaaagcccccaaactcctcatctatggtaacaacaatcggccctcaggggtccctgaccgattctctggctctaagtctggcacctcagtctccctggccatcactgggctccaggctgaggatgaggctgattattactgccagtcatatgacagcagcctgaatggttgggtgttcggcggagggaccaggttgaccgtcctaa

SEQ ID NO: 360 - protein, 8 aa, Artificial Sequence
  source: /note=“Description of Artificial Sequence Synthetic peptide”
GFTFGSYE

SEQ ID NO: 361 - protein, 11 aa, Artificial Sequence
  source: /note=“Description of Artificial Sequence Synthetic peptide”
QSYDNSLNGWV

SEQ ID NO: 362 - DNA, 24 bp, Artificial Sequence
  source: /note=“Description of Artificial Sequence Synthetic oligonucleotide”
ggattcaccttcggaagctatgaa

SEQ ID NO: 363 - DNA, 24 bp, Artificial Sequence
  source: /note=“Description of Artificial Sequence Synthetic oligonucleotide”
attgactttactggttcaaccatc

SEQ ID NO: 364 - DNA, 48 bp, Artificial Sequence
  source: /note=“Description of Artificial Sequence Synthetic oligonucleotide”
gtgagagatgcgggccgctggggcaccagttggtattactttgactat

SEQ ID NO: 365 - DNA, 9 bp, Artificial Sequence
  source: /note=“Description of Artificial Sequence Synthetic oligonucleotide”
ggcaacaac

SEQ ID NO: 366 - DNA, 33 bp, Artificial Sequence
  source: /note=“Description of Artificial Sequence Synthetic oligonucleotide”
cagtcctatgacaacagcctgaatggttgggtg

SEQ ID NO: 367 - protein, 123 aa, Artificial Sequence
  source: /note=“Description of Artificial Sequence Synthetic polypeptide”
AVRLVESGGGLAQPGRSLRLSCQVSGFTFGSYEMNWVRQAPGKGLEWIAYIDFTGSTIYYADSVKGRFTISRNTARNSLYLQMNSLRVEDTAVYYCVRDAGRWGTSWYYFDYWGQGTRVTVSP

SEQ ID NO: 368 - protein, 111 aa, Artificial Sequence
  source: /note=“Description of Artificial Sequence Synthetic polypeptide”
QSVLTQPPSVSGAPGQRVTISCTGISSNIGAGYDIHWYQQIPGKAPKLLVYGNNNRPSGVPDRFSGSKSGTSVSLAITGLQVEDEADYYCQSYDNSLNGWVFGGGTRLTVL

SEQ ID NO: 369 - DNA, 370 bp, Artificial Sequence
  source: /note=“Description of Artificial Sequence Synthetic polynucleotide”
gcggtgcggctggtggagtctgggggaggcttggcacagcctggacggtccctgagactctcgtgtcaagtgtctggattcaccttcggaagctatgaaatgaactgggtccgccaggctcccggcaagggactggagtggattgcctacattgactttactggttcaaccatctactacgcagactctgtgaagggccgattcaccatatccagaaacaccgccaggaactcactctatctgcagatgaacagcctgagagtcgaggacacggctgtttattactgtgtgagagatgcgggccgctggggcaccagttggtattactttgactattggggccaaggaacccgggtcaccgtctccccag

SEQ ID NO: 370 - DNA, 334 bp, Artificial Sequence
  source: /note=“Description of Artificial Sequence Synthetic polynucleotide”
cagtctgtgctgacgcagccgccctcagtgtctggggccccagggcagagggtcaccatctcctgcactgggatcagctccaacatcggggcaggttatgatatacactggtatcagcagattccaggaaaagcccccaaactcctcgtctatggcaacaacaatcggccctcaggagtccctgaccgattctctggctctaagtctggcacctcagtctccctggccatcactgggctccaggttgaggatgaggctgattattactgccagtcctatgacaacagcctgaatggttgggtgttcggcggagggaccaggttgaccgtcctaa